SEQUENCE LISTING (237 sequences)

SEQ ID NO: 1 - DNA, 540 bp, Homo sapiens
atgcagatggttgtgctcccttgcctgggttttaccctgcttctctggagccaggtatcaggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctctga

SEQ ID NO: 2 - DNA, 621 bp, Homo sapiens
atgaattttcaacagaggctgcaaagcctgtggactttagccagacccttctgccctcctttgctggcgacagcctctcaaatgcagatggttgtgctcccttgcctgggttttaccctgcttctctggagccaggtatcaggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctctga

SEQ ID NO: 3 - DNA, 381 bp, Homo sapiens
atgcagatggttgtgctcccttgcctgggttttaccctgcttctctggagccaggtatcaggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcgcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctctga

SEQ ID NO: 4 - DNA, 44 bp, Homo sapiens
atgaattttcaacagaggctgcaaagcctgtggactttagccag

SEQ ID NO: 5 - DNA, 159 bp, Homo sapiens
gatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagt

SEQ ID NO: 6 - protein, 179 aa, Homo sapiens
MQMVVLPCLGFTLLLWSQVSGAQGQEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 7 - protein, 206 aa, Homo sapiens
MNFQQRLQSLWTLARPFCPPLLATASQMQMVVLPCLGFTLLLWSQVSGAQGQEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 8 - protein, 126 aa, Homo sapiens
MQMVVLPCLGFTLLLWSQVSGAQGQEFHFGPCQVKGVVPQKLWEAFWAVKDTMQAQDNITSARLLQQEVLQNVSQENEMFSIRDSAHRRFLLFRRAFKQLDVEAALTKALGEVDILLTWMQKFYKL

SEQ ID NO: 9 - protein, 14 aa, Homo sapiens
MNFQQRLQSLWTLA

SEQ ID NO: 10 - protein, 53 aa, Homo sapiens
DAESCYLVHTLLEFYLKTVFKNYHNRTVEVRTLKSFSTLANNFVLIVSQLQPS

SEQ ID NO: 11 - DNA, 807 bp, Homo sapiens
tgacactatagaccctggcttgcctgcaaacctttacttctgaaatgacttccacgtctgggacgggaaccttccacccacagctatgcctctgattggtgaatggtgaaggtgcctgtctaacttttctgtaaaaagaaccagctgcctccaggcagccagccctcaagcatcacttacaggaccagagcagacccttctgccctcctttgctggcgacagcctctcaaatgcagatggttgtgctcccttgcctgggttttaccctgcttctctggagccaggtatcaggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctctgaatgtctagaccaggacctccctccccctggcactggg

SEQ ID NO: 12 - DNA, 1393 bp, Homo sapiens
cttgcctgcaaacctttacttctgaaatgacttccacggctgggacgggaaccttccacccacagctatgcctctgattggtgaatggtgaaggtgcctgtctaacttttctgtaaaaagaaccagctgcctccaggcagccagccctcaagcatcacttacaggaccagagggacaagacatgactgtgatgaggagctgctttcgccaatttaacaccaagaagaattgaggctgcttgggaggaaggccaggaggaacacgagactgagagatgaattttcaacagaggctgcaaagcctgtggactttagccagacccttctgccctcctttgctggcgacagcctctcaaatgcagatggttgtgctcccttgcctgggttttaccctgcttctctggagccaggtatcaggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctctgaatgtctagaccaggacctccctccccctggcactggtttgttccctgtgtcatttcaaacagtctcccttcctatgctgttcactggacacttcacgcccttggccatgggtcccattcttggcccaggattattgtcaaagaagtcattctttaagcagcgccagtgacagtcagggaaggtgcctctggatgctgtgaagagtctacagagaagattcttgtatttattacaactctatttaattaatgtcagtatttcaactgaagttctatttatttgtgagactgtaagttacatgaaggcagcagaatattgtgccccatgcttctttacccctcacaatccttgccacagtgtggggcagtggatgggtgcttagtaagtacttaataaactgtggtgctttttttggcctgtctttggattgttaaaaaacagagagggatgcttggatgtaaaactgaacttcagagcatgaaaatcacactgtctgct

SEQ ID NO: 13 - DNA, 731 bp, Homo sapiens
tatagaaccaggcttgcctgcaaacctttacttctgaaatgacttccacggctgggacgggaaccttccacccacagctatgcctctgattggtgaatggtgaaggtgcctgtctaacttttctgtaaaaagaaccagctgcctccaggcagccagccctcaagcatcacttacaggaccagagcagacccttctgccctcctttgctggcgacagcctctcaaatgcagatggttgtgctcccttgcctgggttttaccctgcttctctggagccaggtatcaggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcgcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctctgaatgtctagaccaggacctccctccccctggcactggtttgttccctgtgtcatttcaaacagtctcccttcctatgctgttcactggacacttcacgcccttggccatgggtcccattcttggccc

SEQ ID NO: 14 - DNA, 708 bp, Homo sapiens
atggctatgatggaggtccaggggggacccagcctgggacagacctgcgtgctgatcgtgatcttcacagtgctcctgcagtctctctgtgtggctgtaacttacgtgtactttaccaacgagctgaagcagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaa

SEQ ID NO: 15 - protein, 235 aa, Homo sapiens
MAMMEVQGGPSLGQTCVLIVIFTVLLQSLCVAVTYVYFTNELKQMILRTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 16 - DNA, 809 bp, Homo sapiens
gaccggctgcctggctgacttacagcagtcagactctgacaggatcatggctatgatggaggtccaggggggacccagcctgggacagacctgcgtgctgatcgtgatcttcacagtgctcctgcagtctctctgtgtggctgtaacttacgtgtactttaccaacgagctgaagcagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaactgacctggaaagaaaaagcaataacctcaaagtgactattcagttttcaggatg

SEQ ID NO: 17 - DNA, 570 bp, Homo sapiens
attttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggc

SEQ ID NO: 18 - DNA, 588 bp, Homo sapiens
aacgagctgaagcagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggc

SEQ ID NO: 19 - DNA, 846 bp, Homo sapiens
atggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaa

SEQ ID NO: 20 - DNA, 30 bp, Homo sapiens
gagctgaagcagatgattttgagaacctct

SEQ ID NO: 21 - protein, 190 aa, Homo sapiens
ILRTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 22 - protein, 196 aa, Homo sapiens
NELKQMILRTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 23 - protein, 281 aa, Homo sapiens [sequence omitted]

SEQ ID NO: 24 - protein, 10 aa, Homo sapiens
ELKQMILRTS

SEQ ID NO: 25 - DNA, 1776 bp, Homo sapiens
tttcatttcctcactgactataaaagaatagagaaggaagggcttcagtgaccggctgcctggctgacttacagcagtcagactctgacaggatcatggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaactgacctggaaagaaaaagcaataacctcaaagtgactattcagttttcaggatgatacactatgaagatgtttcaaaaaatctgaccaaaacaaacaaacagaaaacagaaaacaaaaaaacctctatgcaatctgagtagagcagccacaaccaaaaaattctacaacacacactgttctgaaagtgactcacttatcccaagagaatgaaattgctgaaagatctttcaggactctacctcatatcagtttgctagcagaaatctagaagactgtcagcttccaaacattaatgcaatggttaacatcttctgtctttataatctactccttgtaaagactgtagaagaaagagcaacaatccatctctcaagtagtgtatcacagtagtagcctccaggtttccttaagggacaacatccttaagtcaaaagagagaagaggcaccactaaaagatcgcagtttgcctggtgcagtggctcacacctgtaatcccaacattttgggaacccaaggtgggtagatcacgagatcaagagatcaagaccatagtgaccaacatagtgaaaccccatctctactgaaagtacaaaaattagctgggtgtgttggcacatgcctgtagtcccagctacttgagaggctgaggcaagagaattgtttgaacccgggaggcagaggttgcagtgtggtgagatcatgccactacactccagcctggcgacagagcgagacttggtttcaaaaaaaaaaaaaaaaaaaacttcagtaagtacgtgttatttttttcaataaaattctattacagtatgtcaaaaaaaaaaaaaaaaaa

SEQ ID NO: 26 - protein, 23 aa, Homo sapiens
MKTCWKIPVFFFVCSFLEPWASA

SEQ ID NO: 27 - protein, 17 aa, Homo sapiens
MALLWGLLVLSWSCLQG

SEQ ID NO: 28 - protein, 13 aa, Homo sapiens
MALLWGLLVLSWS

SEQ ID NO: 29 - protein, 19 aa, Homo sapiens
MALLWGLLVLSWSCLQGPC

SEQ ID NO: 30 - protein, 16 aa, Homo sapiens
MALLWGLLVLSWSCLQ

SEQ ID NO: 31 - protein, 15 aa, Homo sapiens
MALLWGLLVLSWSCL

SEQ ID NO: 32 - protein, 30 aa, Homo sapiens
MDSMPEPASRCLLLLPLLLLLLLLLPAPEL

SEQ ID NO: 33 - protein, 25 aa, Homo sapiens
MDSMPEPASRCLLLLPLLLLLLLLL

SEQ ID NO: 34 - protein, 33 aa, Homo sapiens
MDSMPEPASRCLLLLPLLLLLLLLLPAPELGPS

SEQ ID NO: 35 - protein, 24 aa, Homo sapiens
MDSMPEPASRCLLLLPLLLLLLLL

SEQ ID NO: 36 - protein, 26 aa, Homo sapiens
MDSMPEPASRCLLLLPLLLLLLLLLP

SEQ ID NO: 37 - protein, 32 aa, Homo sapiens
MDSMPEPASRCLLLLPLLLLLLLLLPAPELGP

SEQ ID NO: 38 - protein, 27 aa, Homo sapiens
MDSMPEPASRCLLLLPLLLLLLLLLPA

SEQ ID NO: 39 - protein, 23 aa, Homo sapiens
MDSMPEPASRCLLLLPLLLLLLL

SEQ ID NO: 40 - protein, 35 aa, Homo sapiens
MDSMPEPASRCLLLLPLLLLLLLLLPAPELGPSQA

SEQ ID NO: 41 - protein, 24 aa, Homo sapiens
MRRMWATQGLAVALALSVLPGSRA

SEQ ID NO: 42 - protein, 21 aa, Homo sapiens
MRRMWATQGLAVALALSVLPG

SEQ ID NO: 43 - protein, 19 aa, Homo sapiens
MDLRQFLMCLSLCTAFALS

SEQ ID NO: 44 - protein, 15 aa, Homo sapiens
MDLRQFLMCLSLCTA

SEQ ID NO: 45 - protein, 17 aa, Homo sapiens
MDLRQFLMCLSLCTAFA

SEQ ID NO: 46 - protein, 23 aa, Homo sapiens
MKTCWKIPVFFFVCSFLEPWASA

SEQ ID NO: 47 - protein, 21 aa, Homo sapiens
MMLRVLVGAVLPAMLLAAPPP

SEQ ID NO: 48 - protein, 17 aa, Homo sapiens
MMLRVLVGAVLPAMLLA

SEQ ID NO: 49 - protein, 19 aa, Homo sapiens
MRLSLPLLLLLLGAWAIPG

SEQ ID NO: 50 - protein, 22 aa, Homo sapiens
MRLSLPLLLLLLGAWAIPGGLG

SEQ ID NO: 51 - protein, 18 aa, Homo sapiens
MRLSLPLLLLLLGAWAIP

SEQ ID NO: 52 - protein, 16 aa, Homo sapiens
MRLSLPLLLLLLGAWA

SEQ ID NO: 53 - protein, 14 aa, Homo sapiens
MRLSLPLLLLLLGA

SEQ ID NO: 54 - protein, 20 aa, Homo sapiens
MSMLVVFLLLWGVTWGPVTE

SEQ ID NO: 55 - protein, 16 aa, Homo sapiens
MSMLVVFLLLWGVTWG

SEQ ID NO: 56 - protein, 21 aa, Homo sapiens
MSMLVVFLLLWGVTWGPVTEA

SEQ ID NO: 57 - protein, 24 aa, Homo sapiens
MRALRDRAGLLLCVLLLAALLEAA

SEQ ID NO: 58 - protein, 20 aa, Homo sapiens
MRALRDRAGLLLCVLLLAAL

SEQ ID NO: 59 - protein, 26 aa, Homo sapiens
MRALRDRAGLLLCVLLLAALLEAALG

SEQ ID NO: 60 - protein, 21 aa, Homo sapiens
MRALRDRAGLLLCVLLLAALL

SEQ ID NO: 61 - protein, 23 aa, Homo sapiens
MRALRDRAGLLLCVLLLAALLEA

SEQ ID NO: 62 - protein, 24 aa, Homo sapiens
MRAPGCGRLVLPLLLLAAAALAEG

SEQ ID NO: 63 - protein, 19 aa, Homo sapiens
MRAPGCGRLVLPLLLLAAA

SEQ ID NO: 64 - protein, 22 aa, Homo sapiens
MRAPGCGRLVLPLLLLAAAALA

SEQ ID NO: 65 - protein, 20 aa, Homo sapiens
MRAPGCGRLVLPLLLLAAAA

SEQ ID NO: 66 - protein, 26 aa, Homo sapiens
MRAPGCGRLVLPLLLLAAAALAEGDA

SEQ ID NO: 67 - protein, 21 aa, Homo sapiens
MRAPGCGRLVLPLLLLAAAAL

SEQ ID NO: 68 - protein, 24 aa, Homo sapiens
MRLRRLALFPGVALLLAAGRLVAA

SEQ ID NO: 69 - protein, 23 aa, Homo sapiens
MRLRRLALFPGVALLLAAGRLVA

SEQ ID NO: 70 - protein, 24 aa, Homo sapiens
MPSSVSWGILLLAGLCCLVPVSLA

SEQ ID NO: 71 - protein, 18 aa, Homo sapiens
MPSSVSWGILLLAGLCCL

SEQ ID NO: 72 - protein, 23 aa, Homo sapiens
MPSSVSWGILLLAGLCCLVPVSL

SEQ ID NO: 73 - protein, 17 aa, Homo sapiens
MPSSVSWGILLLAGLCC

SEQ ID NO: 74 - protein, 23 aa, Homo sapiens
MQSLMQAPLLIALGLLLATPAQA

SEQ ID NO: 75 - protein, 18 aa, Homo sapiens
MQSLMQAPLLIALGLLLA

SEQ ID NO: 76 - protein, 25 aa, Homo sapiens
MQSLMQAPLLIALGLLLATPAQAHL

SEQ ID NO: 77 - protein, 20 aa, Homo sapiens
MQSLMQAPLLIALGLLLATP

SEQ ID NO: 78 - protein, 21 aa, Homo sapiens
MQSLMQAPLLIALGLLLATPA

SEQ ID NO: 79 - protein, 23 aa, Homo sapiens
MGRPLHLVLLSASLAGLLLLGES

SEQ ID NO: 80 - protein, 19 aa, Homo sapiens
MGRPLHLVLLSASLAGLLL

SEQ ID NO: 81 - protein, 20 aa, Homo sapiens
MGRPLHLVLLSASLAGLLLL

SEQ ID NO: 82 - protein, 15 aa, Homo sapiens
MGRPLHLVLLSASLA

SEQ ID NO: 83 - protein, 21 aa, Homo sapiens
MGRPLHLVLLSASLAGLLLLG

SEQ ID NO: 84 - protein, 17 aa, Homo sapiens
MGRPLHLVLLSASLAGL

SEQ ID NO: 85 - protein, 18 aa, Homo sapiens
MRIAVICFCLLGITCAIP

SEQ ID NO: 86 - protein, 16 aa, Homo sapiens
MRIAVICFCLLGITCA

SEQ ID NO: 87 - protein, 15 aa, Homo sapiens
MRIAVICFCLLGITC

SEQ ID NO: 88 - protein, 16 aa, Homo sapiens
MKRVLVLLLAVAFGHA

SEQ ID NO: 89 - protein, 14 aa, Homo sapiens
MKRVLVLLLAVAFG

SEQ ID NO: 90 - protein, 25 aa, Homo sapiens
MNSFSTSAFGPVAFSLGLLLVLPAA

SEQ ID NO: 91 - protein, 24 aa, Homo sapiens
MNSFSTSAFGPVAFSLGLLLVLPA

SEQ ID NO: 92 - protein, 27 aa, Homo sapiens
MNSFSTSAFGPVAFSLGLLLVLPAAFP

SEQ ID NO: 93 - protein, 18 aa, Homo sapiens
MALSWVLTVLSLLPLLEA

SEQ ID NO: 94 - protein, 19 aa, Homo sapiens
MARVLGAPVALGLWSLCWS

SEQ ID NO: 95 - protein, 25 aa, Homo sapiens
MARVLGAPVALGLWSLCWSLAIATP

SEQ ID NO: 96 - protein, 21 aa, Homo sapiens
MARVLGAPVALGLWSLCWSLA

SEQ ID NO: 97 - protein, 23 aa, Homo sapiens
MARVLGAPVALGLWSLCWSLAIA

SEQ ID NO: 98 - protein, 31 aa, Homo sapiens
MARVLGAPVALGLWSLCWSLAIATPLPPTSA

SEQ ID NO: 99 - protein, 26 aa, Homo sapiens
MDYYRKYAAIFLVTLSVFLHVLHSAP

SEQ ID NO: 100 - protein, 24 aa, Homo sapiens
MDYYRKYAAIFLVTLSVFLHVLHS

SEQ ID NO: 101 - protein, 18 aa, Homo sapiens
MKLITILFLCSRLLLSLT

SEQ ID NO: 102 - protein, 19 aa, Homo sapiens
MKLITILFLCSRLLLSLTQ

SEQ ID NO: 103 - protein, 16 aa, Homo sapiens
MKLITILFLCSRLLLS

SEQ ID NO: 104 - protein, 23 aa, Homo sapiens
MKLITILFLCSRLLLSLTQESQS

SEQ ID NO: 105 - protein, 17 aa, Homo sapiens
MLRRALLCLAVAALVRA

SEQ ID NO: 106 - protein, 23 aa, Homo sapiens
MPRSCCSRSGALLLALLLQASME

SEQ ID NO: 107 - protein, 26 aa, Homo sapiens
MPRSCCSRSGALLLALLLQASMEVRG

SEQ ID NO: 108 - protein, 23 aa, Homo sapiens
MATHHTLWMGLALLGVLGDLQAA

SEQ ID NO: 109 - protein, 22 aa, Homo sapiens
MATHHTLWMGLALLGVLGDLQA

SEQ ID NO: 110 - protein, 18 aa, Homo sapiens
MATHHTLWMGLALLGVLG

SEQ ID NO: 111 - protein, 18 aa, Homo sapiens
MVRMVPVLLSLLLLLGPA

SEQ ID NO: 112 - protein, 20 aa, Homo sapiens
MVRMVPVLLSLLLLLGPAVP

SEQ ID NO: 113 - protein, 21 aa, Homo sapiens
MVRMVPVLLSLLLLLGPAVPQ

SEQ ID NO: 114 - protein, 17 aa, Homo sapiens
MVRMVPVLLSLLLLLGP

SEQ ID NO: 115 - protein, 18 aa, Homo sapiens
MRSAAVLALLLCAGQVTA

SEQ ID NO: 116 - protein, 15 aa, Homo sapiens
MRSAAVLALLLCAGQ

SEQ ID NO: 117 - protein, 14 aa, Homo sapiens
MRSAAVLALLLCAG

SEQ ID NO: 118 - protein, 26 aa, Homo sapiens
MARSNLPLALGLALVAFCLLALPRDA

SEQ ID NO: 119 - protein, 18 aa, Homo sapiens
MARSNLPLALGLALVAFC

SEQ ID NO: 120 - protein, 20 aa, Homo sapiens
MARSNLPLALGLALVAFCLL

SEQ ID NO: 121 - protein, 28 aa, Homo sapiens
MARSNLPLALGLALVAFCLLALPRDARA

SEQ ID NO: 122 - protein, 21 aa, Homo sapiens
MARSNLPLALGLALVAFCLLA

SEQ ID NO: 123 - protein, 23 aa, Homo sapiens
MARSNLPLALGLALVAFCLLALP

SEQ ID NO: 124 - protein, 22 aa, Homo sapiens
MMKTLLLFVGLLLTWESGQVLG

SEQ ID NO: 125 - protein, 18 aa, Homo sapiens
MMKTLLLFVGLLLTWESG

SEQ ID NO: 126 - protein, 14 aa, Homo sapiens
MMKTLLLFVGLLLT

SEQ ID NO: 127 - protein, 24 aa, Homo sapiens
MDGAMGPRGLLLCMYLVSLLILQA

SEQ ID NO: 128 - protein, 29 aa, Homo sapiens
MDGAMGPRGLLLCMYLVSLLILQAMPALG

SEQ ID NO: 129 - protein, 30 aa, Homo sapiens
MDGAMGPRGLLLCMYLVSLLILQAMPALGS

SEQ ID NO: 130 - protein, 23 aa, Homo sapiens
MDGAMGPRGLLLCMYLVSLLILQ

SEQ ID NO: 131 - protein, 31 aa, Homo sapiens
MDGAMGPRGLLLCMYLVSLLILQAMPALGSA

SEQ ID NO: 132 - protein, 22 aa, Homo sapiens
MKVLAAGVVPLLLVLHWKHGAG

SEQ ID NO: 133 - protein, 23 aa, Homo sapiens
MKVLAAGVVPLLLVLHWKHGAGS

SEQ ID NO: 134 - protein, 18 aa, Homo sapiens
MSLSAFTLFLALIGGTSG

SEQ ID NO: 135 - protein, 15 aa, Homo sapiens
MSLSAFTLFLALIGG

SEQ ID NO: 136 - protein, 17 aa, Homo sapiens
MSLSAFTLFLALIGGTS

SEQ ID NO: 137 - protein, 14 aa, Homo sapiens
MSLSAFTLFLALIG

SEQ ID NO: 138 - protein, 18 aa, Homo sapiens
MKLLMVLMLAALSQHCYA

SEQ ID NO: 139 - protein, 30 aa, Homo sapiens
MQSVQSTSFCLRKQCLCLTFLLLHLLGQVA

SEQ ID NO: 140 - protein, 32 aa, Homo sapiens
MQSVQSTSFCLRKQCLCLTFLLLHLLGQVAAT

SEQ ID NO: 141 - protein, 28 aa, Homo sapiens
MQSVQSTSFCLRKQCLCLTFLLLHLLGQ

SEQ ID NO: 142 - protein, 27 aa, Homo sapiens
MQSVQSTSFCLRKQCLCLTFLLLHLLG

SEQ ID NO: 143 - protein, 31 aa, Homo sapiens
MQSVQSTSFCLRKQCLCLTFLLLHLLGQVAA

SEQ ID NO: 144 - protein, 26 aa, Homo sapiens
MARGGRGRRLGLALGLLLALVLAPRV

SEQ ID NO: 145 - protein, 22 aa, Homo sapiens
MARGGRGRRLGLALGLLLALVL

SEQ ID NO: 146 - protein, 29 aa, Homo sapiens
MARGGRGRRLGLALGLLLALVLAPRVLRA

SEQ ID NO: 147 - protein, 24 aa, Homo sapiens
MARGGRGRRLGLALGLLLALVLAP

SEQ ID NO: 148 - protein, 23 aa, Homo sapiens
MARGGRGRRLGLALGLLLALVLA

SEQ ID NO: 149 - protein, 25 aa, Homo sapiens
MRLGPRTAALGLLLLCAAAAGAGKA

SEQ ID NO: 150 - protein, 19 aa, Homo sapiens
MRLGPRTAALGLLLLCAAA

SEQ ID NO: 151 - protein, 22 aa, Homo sapiens
MRLGPRTAALGLLLLCAAAAGA

SEQ ID NO: 152 - protein, 18 aa, Homo sapiens
MRLGPRTAALGLLLLCAA

SEQ ID NO: 153 - protein, 20 aa, Homo sapiens
MRLGPRTAALGLLLLCAAAA

SEQ ID NO: 154 - protein, 21 aa, Homo sapiens
MRLGPRTAALGLLLLCAAAAG

SEQ ID NO: 155 - protein, 23 aa, Homo sapiens
MRLGPRTAALGLLLLCAAAAGAG

SEQ ID NO: 156 - protein, 19 aa, Homo sapiens
MAWFALYLLSLLWATAGTS

SEQ ID NO: 157 - protein, 18 aa, Homo sapiens
MAWFALYLLSLLWATAGT

SEQ ID NO: 158 - protein, 20 aa, Homo sapiens
MAWFALYLLSLLWATAGTST

SEQ ID NO: 159 - protein, 24 aa, Homo sapiens
MAWFALYLLSLLWATAGTSTQTQS

SEQ ID NO: 160 - protein, 16 aa, Homo sapiens
MAWFALYLLSLLWATA

SEQ ID NO: 161 - protein, 17 aa, Homo sapiens
MAWFALYLLSLLWATAG

SEQ ID NO: 162 - protein, 14 aa, Homo sapiens
MAWFALYLLSLLWA

SEQ ID NO: 163 - protein, 17 aa, Homo sapiens
MHLLLFQLLVLLPLGKT

SEQ ID NO: 164 - protein, 18 aa, Homo sapiens
MPLGLLWLGLALLGALHA

SEQ ID NO: 165 - protein, 20 aa, Homo sapiens
MPLGLLWLGLALLGALHAQA

SEQ ID NO: 166 - protein, 15 aa, Homo sapiens
MPLGLLWLGLALLGA

SEQ ID NO: 167 - protein, 29 aa, Homo sapiens
MRHNWTPDLSPLWVLLLCAHVVTLLVRAT

SEQ ID NO: 168 - protein, 24 aa, Homo sapiens
MRHNWTPDLSPLWVLLLCAHVVTL

SEQ ID NO: 169 - protein, 28 aa, Homo sapiens
MRHNWTPDLSPLWVLLLCAHVVTLLVRA

SEQ ID NO: 170 - protein, 19 aa, Homo sapiens
MLPLTMTVLILLLLPTGQA

SEQ ID NO: 171 - protein, 18 aa, Homo sapiens
MLPLTMTVLILLLLPTGQ

SEQ ID NO: 172 - protein, 21 aa, Homo sapiens
MLPLTMTVLILLLLPTGQAAP

SEQ ID NO: 173 - protein, 15 aa, Homo sapiens
MTSILTVLICLGLSL

SEQ ID NO: 174 - protein, 26 aa, Homo sapiens
MENPSPAAALGKALCALLLATLGAAG

SEQ ID NO: 175 - protein, 25 aa, Homo sapiens
MENPSPAAALGKALCALLLATLGAA

SEQ ID NO: 176 - protein, 24 aa, Homo sapiens
MENPSPAAALGKALCALLLATLGA

SEQ ID NO: 177 - protein, 28 aa, Homo sapiens
MENPSPAAALGKALCALLLATLGAAGQP

SEQ ID NO: 178 - protein, 20 aa, Homo sapiens
MKGWGWLALLLGALLGTAWA

SEQ ID NO: 179 - protein, 18 aa, Homo sapiens
MKGWGWLALLLGALLGTA

SEQ ID NO: 180 - protein, 16 aa, Homo sapiens
MKGWGWLALLLGALLG

SEQ ID NO: 181 - protein, 24 aa, Homo sapiens
MRAPGCGRLVLPLLLLAAAALAEG

SEQ ID NO: 182 - protein, 19 aa, Homo sapiens
MRAPGCGRLVLPLLLLAAA

SEQ ID NO: 183 - protein, 22 aa, Homo sapiens
MRAPGCGRLVLPLLLLAAAALA

SEQ ID NO: 184 - protein, 20 aa, Homo sapiens
MRAPGCGRLVLPLLLLAAAA

SEQ ID NO: 185 - protein, 26 aa, Homo sapiens
MRAPGCGRLVLPLLLLAAAALAEGDA

SEQ ID NO: 186 - protein, 21 aa, Homo sapiens
MRAPGCGRLVLPLLLLAAAAL

SEQ ID NO: 187 - protein, 18 aa, Homo sapiens
MSLGQSACLFLSIARSRS

SEQ ID NO: 188 - protein, 18 aa, Homo sapiens
MQRLGATLLCLLLAAAVP

SEQ ID NO: 189 - protein, 19 aa, Homo sapiens
MQRLGATLLCLLLAAAVPT

SEQ ID NO: 190 - protein, 22 aa, Homo sapiens
MQRLGATLLCLLLAAAVPTAPA

SEQ ID NO: 191 - protein, 16 aa, Homo sapiens
MQRLGATLLCLLLAAA

SEQ ID NO: 192 - protein, 21 aa, Homo sapiens
MQRLGATLLCLLLAAAVPTAP

SEQ ID NO: 193 - protein, 26 aa, Homo sapiens
MSASKIPLFKMKDLILILCLLEMSFA

SEQ ID NO: 194 - protein, 28 aa, Homo sapiens
MSASKIPLFKMKDLILILCLLEMSFAVP

SEQ ID NO: 195 - protein, 18 aa, Homo sapiens
MEMFQGLLLLLLLSMGGT

SEQ ID NO: 196 - protein, 20 aa, Homo sapiens
MEMFQGLLLLLLLSMGGTWA

SEQ ID NO: 197 - protein, 16 aa, Homo sapiens
MEMFQGLLLLLLLSMG

SEQ ID NO: 198 - protein, 18 aa, Homo sapiens
MTMRSLLRTPFLCGLLWA

SEQ ID NO: 199 - protein, 20 aa, Homo sapiens
MTMRSLLRTPFLCGLLWAFC

SEQ ID NO: 200 - protein, 26 aa, Homo sapiens
MTMRSLLRTPFLCGLLWAFCAPGARA

SEQ ID NO: 201 - protein, 23 aa, Homo sapiens
MTMRSLLRTPFLCGLLWAFCAPG

SEQ ID NO: 202 - protein, 13 aa, Homo sapiens
MLLLLTLALLGGP

SEQ ID NO: 203 - protein, 16 aa, Homo sapiens
MLLLLTLALLGGPTWA

SEQ ID NO: 204 - protein, 14 aa, Homo sapiens
MLLLLTLALLGGPT

SEQ ID NO: 205 - protein, 17 aa, Homo sapiens
MLLLLTLALLGGPTWAG

SEQ ID NO: 206 - protein, 25 aa, Homo sapiens
MEAAPSRFMFLLFLLTCELAAEVAA

SEQ ID NO: 207 - protein, 21 aa, Homo sapiens
MEAAPSRFMFLLFLLTCELAA

SEQ ID NO: 208 - protein, 18 aa, Homo sapiens
MPPFLLLTCLFITGTSVS

SEQ ID NO: 209 - protein, 16 aa, Homo sapiens
MPPFLLLTCLFITGTS

SEQ ID NO: 210 - protein, 15 aa, Homo sapiens
MPPFLLLTCLFITGT

SEQ ID NO: 211 - protein, 21 aa, Homo sapiens
MGPVRLGILLFLFLAVHEAWA

SEQ ID NO: 212 - protein, 29 aa, Homo sapiens
MRHNWTPDLSPLWVLLLCAHVVTLLVRAT

SEQ ID NO: 213 - protein, 24 aa, Homo sapiens
MRHNWTPDLSPLWVLLLCAHVVTL

SEQ ID NO: 214 - protein, 28 aa, Homo sapiens
MRHNWTPDLSPLWVLLLCAHVVTLLVRA

SEQ ID NO: 215 - protein, 23 aa, Homo sapiens
MEICRGLRSHLITLLLFLFHSET

SEQ ID NO: 216 - protein, 25 aa, Homo sapiens
MEICRGLRSHLITLLLFLFHSETIC

SEQ ID NO: 217 - protein, 32 aa, Homo sapiens
MWAPRCRRFWSRWEQVAALLLLLLLLGVPPRS

SEQ ID NO: 218 - protein, 34 aa, Homo sapiens
MWAPRCRRFWSRWEQVAALLLLLLLLGVPPRSLA

SEQ ID NO: 219 - protein, 29 aa, Homo sapiens
MWAPRCRRFWSRWEQVAALLLLLLLLGVP

SEQ ID NO: 220 - protein, 30 aa, Homo sapiens
MWAPRCRRFWSRWEQVAALLLLLLLLGVPP

SEQ ID NO: 221 - protein, 27 aa, Homo sapiens
MWAPRCRRFWSRWEQVAALLLLLLLLG

SEQ ID NO: 222 - protein, 26 aa, Homo sapiens
MWCASPVAVVAFCAGLLVSHPVLTQG

SEQ ID NO: 223 - protein, 24 aa, Homo sapiens
MWCASPVAVVAFCAGLLVSHPVLT

SEQ ID NO: 224 - DNA, 24 bp, Artificial Sequence
  Description of Artificial Sequence Synthetic primer
aacgagctgaagcagatgattttg

SEQ ID NO: 225 - DNA, 23 bp, Artificial Sequence
  Description of Artificial Sequence Synthetic primer
ttagccaactaaaaaggccccga

SEQ ID NO: 226 - DNA, 21 bp, Artificial Sequence
  Description of Artificial Sequence Synthetic primer
gccaactaaaaaggccccgaa

SEQ ID NO: 227 - DNA, 23 bp, Artificial Sequence
  Description of Artificial Sequence Synthetic primer
attttgagaacctctgaggaaac

SEQ ID NO: 228 - DNA, 22 bp, Artificial Sequence
  Description of Artificial Sequence Synthetic primer
gtgagagaaagaggtcctcaga

SEQ ID NO: 229 - DNA, 19 bp, Artificial Sequence
  Description of Artificial Sequence Synthetic primer
ccacccaccaccaccaatg

SEQ ID NO: 230 - DNA, 34 bp, Artificial Sequence
  Description of Artificial Sequence Synthetic primer
ggtgacactatagaactcacctatctccccaaca

SEQ ID NO: 231 - DNA, 21 bp, Artificial Sequence
  Description of Artificial Sequence Synthetic primer
gggcccctggaacagaacttc

SEQ ID NO: 232 - DNA, 21 bp, Artificial Sequence
  Description of Artificial Sequence Synthetic primer
gcgtagcatttaggtgacact

SEQ ID NO: 233 - protein, 168 aa, Homo sapiens
VRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 234 - DNA, 84 bp, Artificial Sequence
  Description of Artificial Sequence Synthetic nucleotide sequence
gccgccaccatgaagacctgctggaaaattccagttttcttctttgtgtgcagtttcctggaaccctgggcatctgcagaattc

SEQ ID NO: 235 - DNA, 123 bp, Artificial Sequence
  Description of Artificial Sequence Synthetic nucleotide sequence
ggatccctggttccgcgtggctcaggctcattcgaaggtaagcctatccctaaccctctcctcggtctcgattctacgcgtaccggtcatcatcaccatcaccatcaccatggaggacagtga

SEQ ID NO: 236 - protein, 6 aa, Artificial Sequence
  Description of Artificial Sequence Synthetic 6xHis tag
HHHHHH

SEQ ID NO: 237 - protein, 8 aa, Artificial Sequence
  Description of Artificial Sequence Synthetic 8xHis tag
HHHHHHHH

(19) United States
(12) Patent Application Publication
(10) Pub. No.: US 2008/0242603 A1
(43) Pub. Date: Oct. 2, 2008

(54) Novel Apo2L and IL-24 Polypeptides, Polynucleotides, and Methods of Their Use

(76) Inventors: Yan Wang, Redwood City, CA (US); Amy L. Tsui Collins, Oakland, CA (US); Kevin Hestir, Kensington, CA (US); Ernestine Lee, Kensington, CA (US); Forgan Robert Halenbeck, San Rafael, CA (US); Elizabeth Bosch, Cupertino, CA (US); Thomas Linnemann, San Francisco, CA (US); Lewis Thomas Williams, Mill Valley, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/589,561

(22) PCT Filed: Feb. 18, 2005
(86) PCT No.: PCT/US2005/005221

§ 371 Date: May 20, 2008

Related U.S. Application Data
(60) Provisional application No. 60/546,385, filed on Feb. 20, 2004.
(60) Provisional application No. 60/647,013, filed on Jan. 27, 2005.
(60) Provisional application No. 60/654,229, filed on Feb. 18, 2005.

(51) Int. Cl. A61K 38/17; C07H 21/00; C07K 14/00; C12N 5/06; C12N 15/09; A61P 35/00

(52) U.S. Cl. 514/12; 536/23.5; 530/350; 435/320.1; 435/348; 435/455; 530/333

(57) ABSTRACT

Disclosed are newly identified Interleukin 24 and APO2L splice variant molecules, their polypeptide sequences, and the polynucleotides encoding the polypeptide sequences. Also provided is a procedure for producing such polypeptides by recombinant techniques employing, for example, vectors and host cells, and, for example, heterologous secretory leader sequences. Also disclosed are methods for using such polypeptides and modulators thereof for the treatment of diseases, including cancer, immune diseases, infectious diseases, and ischemic diseases.

### PRIORITY CLAIM

This application claims the benefit of provisional applications 60/546,385, filed on Feb. 20, 2004; 60/647,013, filed on Jan. 27, 2005, and the U.S. Provisional Application “Fusion Polypeptides of Human Fetuin and Therapeutically Active Polypeptides,” filed Feb. 18, 2005, the disclosures of which are all hereby incorporated by reference.

### FIELD OF THE INVENTION

The present invention relates to newly identified splice variants and fragments thereof, including those of interleukin 24 (IL-24) and APO2 ligand/tumor necrosis factor-related apoptosis-inducing ligand (APO2L/TRAIL), their polypeptide sequences, the polynucleotides encoding the polypeptide sequences, vectors, host cells, and compositions, and kits containing such. The invention further relates to methods of treating diseases using the compositions herein. It also relates to leader sequences useful for the production of secreted forms of IL-24 and APO2L polypeptides, nucleic acid constructs that encode these leader sequences, as well as recombinant host cells and methods of making and using these polypeptides with the leader sequences. It further relates to fusion proteins containing the present polypeptides and modulators of the IL-24 and APO2L polypeptides, their use in the treatment of disease, and methods of identifying and producing these modulators.

### BACKGROUND OF THE INVENTION

The control of cell numbers in mammals is believed to be determined, in part, by a balance between cell proliferation and cell death. One form of cell death, sometimes referred to as necrotic cell death, is typically characterized as pathologic, resulting from trauma or injury. In contrast, there is another physiologic form of cell death that usually proceeds in an orderly or controlled manner. This orderly or controlled form of cell death is often referred to as apoptosis (Barr et al., Bio/Technology, 12:487-493 (1994); Steller et al., Science, 267:1445-1449 (1995)).

Apoptotic cell death naturally occurs in many physiological processes, including embryonic development and clonal selection in the immune system (Itoh et al., Cell, 66:233-243 (1991)). Decreased levels of apoptotic cell death have been associated with a variety of pathological conditions, including cancer, lupus, and herpes virus infection (Thompson, Science, 267:1456-1462 (1995)). Increased levels of apoptotic cell death may be associated with a variety of other pathological conditions, including AIDS, Alzheimer's disease, Parkinson's disease, amyotrophic lateral sclerosis, multiple sclerosis, retinitis pigmentosa, cerebellar degeneration, aplastic anemia, myocardial infarction, stroke, reperfusion injury, and toxin-induced liver disease (Thompson, 1995).

Secreted proteins are likely to function as intercellular communicators of signals by acting as ligands, while their counterpart membrane-associated receptors, having extracellular and intracellular or cytoplasmic domains, transmit extracellular signals into the cell when they bind a ligand on the cell surface. Secreted proteins are typically expressed as full-length polypeptides, sometimes referred to as protein precursors, that are post-translationally processed in the Golgi complex or endoplasmic reticulum (ER) by cleavage of the secretory leader sequences to generate a mature polypeptide or by addition of carbohydrates in a glycosylation process (Hirschberg, Annu. Rev. Biochem. 56:63-87 (1987)).

While receptors have often been considered as important potential therapeutic targets, secreted proteins are of particular interest as potential therapeutic agents. Secreted proteins often have signaling or hormonal functions, and hence high and specific biological activities (Schoen, F. J., Robbins Pathologic Basis of Disease, W.B. Saunders Co., Philadelphia. (1994)). For example, secreted proteins control physiological reactions such as differentiation and proliferation, blood clotting and thrombolysis, somatic growth and cell death, and immune responses (Schoen, F. J., Robbins Pathologic Basis of Disease, W.B. Saunders Co., Philadelphia. (1994)). Significant resources and research efforts have been expended in the discovery and investigation of new secreted proteins that control biological functions. Some of these secreted protein targets, including cytokines and peptide hormones, are manufactured and used as therapeutic agents (Zav'Yalov et al., APMIS 105:161-86 (1997)). However, of the several thousand secreted proteins hypothetically in existence, few are currently used as therapeutic compounds.

Secreted proteins are generally characterized by hydrophobic N-terminal signal peptide (SP) or secretory leader sequences, although some secreted proteins, such as the fibroblast growth factor family, lack such a sequence. The SP is typically about 16 to 30 amino acid residues in length and is usually cleaved by a signal peptidase in the Golgi or the ER lumen before it is exported outside the cell. The resulting mature protein or the actual secreted polypeptide thus lacks the signal peptide sequence.

Naturally occurring secreted proteins are typically expressed in varying amounts depending on their physiological roles in vivo. As a result, many proteins, when expressed under the regulation of their naturally occurring secretory leader sequences, are expressed in quantities too small for commercial purposes. It would be highly desirable, therefore, to be able to produce proteins for therapeutic applications in large quantities, regardless of how they are produced in the natural environment. It would, hence, be advantageous if nucleic acid constructs and methods could be devised to enable increased protein production in vivo or in vitro.

IL-24, also referred to as melanoma differentiation-associated gene 7 (Mda-7), is a cytokine related to the IL-10 family. IL-24 was first identified due to its elevated expression in growth arrested and terminally-differentiated human melanoma cells (Ekmekcioglu et al., Int. J Cancer, 94(1):54-59 (2002); Madireddi et al., Adv. Exp. Med. Biol., 465:239-261 (2000)). APO2L, also known as TRAIL, is a member of the tumor necrosis factor (TNF) cytokine family (Marsters et al., Recent Prog. Horm. Res., 54:225-234 (1999)). Members of the APO2L family have diverse biological effects, including induction of apoptosis, promotion of cell survival, and regulation of the immune system (Di Pietro et al., J. Cell Physiol. 201331-40 (2004)). In a xenograft model of human colon carcinoma, Xiang, et al. Drug Metab. Dispos. 32: 1230-8 (2004) found that kidney contained the highest levels of radiolabled APO2L among the tissues examined.

Both APO2L and IL-24 have been found to be associated with diseases. For example, some diseases are caused by blocking TNF-induced apoptosis, and some are caused by inducing TNF-induced apoptosis. However, the cause or effect of the presence of APO2L and IL-24 in disease is unclear. It would be highly desirable to clarify the function and utility of APO2L and IL-24 molecules, as they may provide an additional means of controlling diseases, as well as providing further insight into the development of self-tolerance by the immune system and the etiology of cancers, immune diseases, infectious diseases, and ischemia-related disorders.

### SUMMARY OF THE INVENTION

The inventors herein have discovered novel splice variants of APO2L and IL-24, and found that certain fragments thereof possess activity of wild-type molecules. For example, the novel splice variants include fragments of APO2L, such as one containing amino acid residues 92-281 (the 92 species), and another containing amino acid residues 40-45 and 92-281 (the 40-42,92-281 species), all amino acid residues being numbered in accordance to the amino acid residue positions of the full-length wild-type APO2L, NM_{—}003801_NM-_{—}03810, with the first amino acid residue at the N-terminus of the wild-type APO2L being amino acid residue number 1. Moreover, the inventors found that the 92 species, the 40-42,92-281 species, and another polypeptide containing amino acid residues 114-281 (the 114 species) all have substantially the same or greater ability to induce apoptosis in cancer cells, such as COLO-205 cells, as 4 ng/ml-100 ng/ml of rhAPO2L. Yet, surprisingly, the 92 species and the 40-42,92-281 species are more sparing of the normal cells, such as normal human hepatocytes and kidney cells and hence, have less cytotoxic or cytostatic effect on normal cells.

The invention provides an isolated nucleic acid molecule with one or more of the nucleotide sequences of SEQ ID NOS.:14, 16, 17, and 18; a polynucleotide encoding a polypeptide comprising a first amino acid sequence of one or more of SEQ ID NOS.:15, 21, and 22; nucleotide sequences complementary to the sequence of SEQ ID NOS.:14, 16, 17, or 18; or a biologically active fragment of any of these.

This isolated nucleic acid molecule can be a cDNA molecule, a genomic DNA molecule, a cRNA molecule, a siRNA molecule, an RNAi molecule, an mRNA molecule, an anti-sense molecule, and/or a ribozyme. It can also be the complement of any of these.

Embodiments of the isolated nucleic acid molecule can be SEQ ID NO.:17 or SEQ ID NO.:18, either in the presence or absence of a second polynucleotide. The second polynucleotide, could, e.g., encode a homologous or heterologous secretory leader, for example, a secretory leader found in SEQ ID NOS.:26-223.

The invention also provides an isolated polypeptide with one or more of the amino acid sequences of SEQ ID NOS.:15, 21, or 22; a sequence encoded by one or more of SEQ ID NOS.:14, 16, 17, or 18; and/or an active fragment of any of these. This isolated polypeptide can be present in a cell culture and/or a cell culture medium. The cell culture can be a bacterial cell culture, a mammalian cell culture, an insect cell culture, or a yeast cell culture. This isolated polypeptide may be found in a plant or a non-human animal. Embodiments of this polypeptide may have the amino acid sequence of SEQ ID NO.:21 or SEQ ID NO.:22. It may further comprise a second amino acid sequence, e.g., either a homologous or a heterologous leader operably linked to the isolated polypeptide. Embodiments of the secretory leader sequence can be a heterologous leader sequence found in SEQ ID NOS.:26-223. The polypeptide may comprise at least six contiguous amino acids from SEQ ID NO.:24 or encoded by SEQ ID NO.:20.

The invention further provides a vector comprising an isolated nucleic acid molecule described above and a promoter that regulates its expression. This vector can be a viral vector or a plasmid, e.g., a pTT vector. The promoter may either be naturally contiguous to the nucleic acid molecule of interest or not naturally contiguous to the nucleic acid molecule of interest. It may be inducible, conditionally-active (such as the cre-lox promoter), constitutive, and/or tissue-specific.

The invention further provides a recombinant host cell with one or more of the nucleic acids, polypeptides, or vectors described above. It may be either a prokaryotic or eukaryotic cell; if eulcaryotic, it may be of human, non-human mammalian, insect, fish, plant, or fungal organ. Suitable mammalian host cells include those of the 293 CHO cell lines, e.g., 293T cells and 293E cells. The invention provides an animal injected with an isolated nucleic acid molecule or polypeptide of the invention, for example, a rodent, a non-human primate, a rabbit, a dog, or a pig.

The invention provides both nucleic acid and polypeptide compositions, each comprising a carrier. They may, for example be provided as vector compositions, and/or host cell compositions. The carrier may be a pharmaceutically acceptable carrier or an excipient.

In another aspect, the invention provides a method of producing a recombinant host cell by providing a vector with an isolated nucleic acid molecule of the invention, and allowing a cell to come into contact with the vector to form a recombinant host cell transfected with the nucleic acid molecule. The invention provides a method of producing polypeptide by providing an isolated nucleic acid of the invention and expressing it in an expression system to produce the polypeptide. Both cell-based and cell-free expression systems can be used to practice the method. Both prokaryotic and eukaryotic expression systems are suitable. For example, the expression system may comprise a host cell transfected with an isolated nucleic acid molecule of the invention, forming a recombinant host cell, which can be cultured. Cell-free expression systems suitable for practicing the method include wheat germ lysate expression systems, rabbit reticulocyte expression systems, ribosomal displays, and E. coli lysate expression systems. The invention provides a polypeptide produced by both cell-based and cell-free expression systems. It provides a polypeptide produced by these systems with mammalian, insect, plant, yeast, or bacterial host cells.

In a further aspect, the invention provides a method of determining the presence of an antibody specific to a polypeptide of the invention in a sample, by providing a composition with the polypeptide, allowing the polypeptide to interact with the sample, and determining whether interaction has occurred between the polypeptide and the antibody, if present, in the sample. The invention provides an antibody specific to a polypeptide of the invention, or a fragment thereof. Antibodies of the invention include polyclonal antibodies, monoclonal antibodies, single chain antibodies, and active fragments of any of these. Antibody fragments of the invention include antigen binding fragments, Fc fragments, cdr fragments, V_{H} fragments, V_{C} fragments, and framework fragments. The invention provides isolated polypeptides, such as those found in the Sequence Listing, encoded by a polynucleotide of the Sequence Listing, and/or produced by any of the methods described above, as well as any biologically active fragment of any of these, wherein the polypeptide further comprises at least one fusion partner. The fusion partner may be, e.g., a polymer, a polypeptide, a succinyl group, fetuin, leucine zipper nuclear factor erythroid derivative-2 (NFE2), neuroretinal leucine zipper, mannose motif (mbp1), tetranectin, an Fc fragment, or serum albumin.

The invention also provides a method of inhibiting tumor growth and/or killing tumor cells by providing a composition of a polypeptide of the invention which includes an active fragment of the polypeptide, and contacting the tumor with this composition.

The tumor cells susceptible to this method include cells with a death domain receptor, with an amino acid sequence of the Sequence Listing, or with an active fragment of any of these. The tumor cells may be human. They may be solid tumor cells or leukemic tumor cells. They may, e.g., be found in a carcinoma, e.g., a mammary adenocarcinoma, or a non-small cell lung carcinoma. They may be found in a breast tumor, a colon tumor, a lung tumor, a prostate tumor, a bladder tumor, a stomach tumor, a glioblastoma, or in skin cancer.

The invention further provides a method for treating proliferative disease in a subject by providing a polypeptide composition with a polypeptide of the Sequence Listing, a polypeptide encoded by a polynucleotide of the Sequence Listing, a polypeptide produced by the methods described herein, a biologically active fragment of any of these, and/or an antibody directed to any of these, and administering the composition to the subject. This method can be used to treat a proliferative disease, such as a mammary adenocarcinoma, non-small cell lung carcinoma, breast tumors, lung tumors, prostate tumors, colon tumors, stomach tumors, bladder tumors, glioblastomas, and/or skin cancer.

The invention provides a pharmaceutical composition comprising an isolated polypeptide chosen as described herein, including an active fragment, an anti-cancer agent, and a pharmaceutically acceptable carrier (Bouralexis et al., Apoptosis 1035-51 (2005). The benefit of combination chemotherapy is consistent with the observation that APO2L activates anti-apoptotic or pro-survival pathways (Zauli et al., J. Cell Phyiol. 202900-11 (2005).

Suitable anti-cancer agents include, but are not limited to, chemotherapeutic agents, radiotherapeutic agents, anti-angiogenic agents, and apoptosis-inducing agents. The chemotherapeutic agent may be any known in the art, e.g., steroid, a cytokine, a cytosine arabinoside, fluorouracil, methotrexate, aminopterin, an anthracycline, mitomycin C, a vinca alkaloid, an antibiotic, demecolcine, etoposide, mithramycin, chlorambucil, and/or melphalan, as well as others described in Kufe et al., eds. (2003) Cancer Medicine 6th ed., B.C. Decker, Inc.

### DESCRIPTION OF THE FIGURES AND TABLES

FIG. 1 shows the polypeptide alignment between the newly identified APO2L splice variant CLN00100891-5pvl.a with the “wild-type” APO2L having 281 amino acid residues and a NCBI accession number of NP-003801_NM_{—}003810. The alignment was performed using Clustal W (1.8). The asterisks (*) indicate shared amino acid residues. The hyphens “-” indicate amino acid residues that are missing from the novel sequence. The numbering convention used in this application in reference to the APO2L splice variant is by reference to the amino acid residue position of the wild type APO2L.

FIG. 2 shows the polypeptide alignment between two newly identified IL-24 splice variants, CLN00493987_{—}5pvl.a and CLN00453866_{—}5pvl.a, with the “wild-type” IL-24 having a NCBI accession number of NP_{—}006841_NM006850. NP_{—}006841_NM006850_exon4 represents exon 4 of this wild-type sequence; and NP006841_NM_{—}006850_exon1 represents exon 1 of the wild-type sequence. The alignment was performed using Clustal Format for T-COFFEE Version_{—}1.37, CPU=0.00 sec, SCORE=66, Nseq=5, Len=206. The hyphens “-” indicate amino acid residues that are missing from the novel sequences.

FIG. 3 shows the polypeptide alignment between the newly identified full length APO2L splice variant (SV), CLN00108891_{—}5pvl.a, and fraginents thereof: CLN00108891_frag1 (the “92-281” construct or the “92” construct) and CLN00108891_frag2 (the “40-45,92-281” construct or the “40” construct) with the full length wild-type APO2L having the NCBI accession number NP_{—}003801_NM003810 and a fragment thereof, NP_{—}003801_NM003810_frag1 (the “114-281” construct or the “114” construct). The alignment was performed using Clustal Format for T-COFFEE Version_{—}1.37, CPU=0.00 sec, SCORE=93, Nseq=5, Len=281. The asterisks (*) indicate shared amino acid residues. The hyphens “-” indicate amino acid residues that are missing.

FIG. 4 shows the APO2L constructs corresponding to the wild-type (WT) and splice variant (SV) and the “114,” “92,” and “40” constructs. “TM” represents the transmembrane domain spanning amino acid residues 17-39, as numbered on the basis of the WT APO2L full length polypeptide sequence.

FIG. 5 shows a plasmid map of the pTT5 vector used to create the expression vector for the secreted APO2L constructs 114, 92, and 40, with or without a cleavable tag. FIG. 5 includes the composition of Vector C, which was inserted into the pTT5 vector as designated at “C.” “Kozak” represents an optional Kozak sequence. “SP” represents an exemplary secretory leader sequence. “EcoR1” and “BamH1” represent restriction sites. The dash lines “ . . . ” between EcoR1 and BamH1 represent the location into which the polynucleotide sequence encoding the 114 construct, the 92 construct, or the 40 construct were inserted. “Thrombin” represents an optional enzymatic cleavage site for the V5H8 tag. “TGA” represents the stop codon. The remaining nucleotide sequences, such as “TTCGAA” and “GGAGGACAG” respresent optional additional random sequences that allow for flexibility of the molecule.

FIG. 6 shows APO2L protein expression by quantitative Western blotting, both in the presence and absence of a tag, as further described in the Examples.

FIG. 7, top panel, is a bar diagram showing the induction of apoptosis in COLO-205 colon cancer cells after incubation with either recombinant human APO2L (rhAPO2L) in conditioned medium (CM) from CHO cells transfected with a vector-only (CHO vector CM) or with conditioned media from cells transfected with the vector containing the 114-281 construct, the 92-281 construct, or the 40-45, 92-281 (40-281) construct. The three constructs in the middle were constructed with tags, while the constructs to the right were constructed without tags. The extent of caspases 3/7 release was measured as an indication of the extent of induction of apoptosis.

FIG. 7, bottom panel, is a bar diagram showing the effect of APO2L containing conditioned media on the proliferation of COLO-205 cells. The conditioned media used are the same as described above, and used in the top panel.

FIG. 8 shows the effect of conditioned media from APO2L transfected cells as described above for FIG. 7 on induction of apoptosis in Hela-229 cervical cancer cells and the effect on proliferation of such Hela cells.

FIG. 9 shows the effect of a 24 hour exposure to the conditioned media, as described above for FIG. 7, on the proliferation of COLO-205 cells, human hepatocytes, and 293 kidney epithelial cells, respectively.

FIG. 10 is a bar diagram showing the effect of conditioned media from cells transfected with different secreted proteins on the proliferation of COLO-205 cells. The assay was conducted in a 96 well plate. APO2L 92-281, APO2L 114-281, and APO2L 40-45/92-281 mark the wells in which conditioned media containing such was present.

FIG. 11 shows a cell proliferation assay conducted as before, but using APO2L fragments made in a cell free expression system.

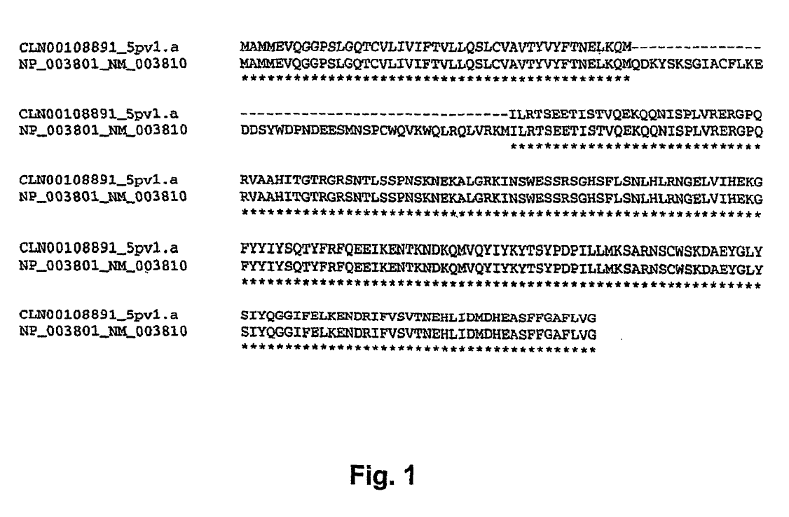

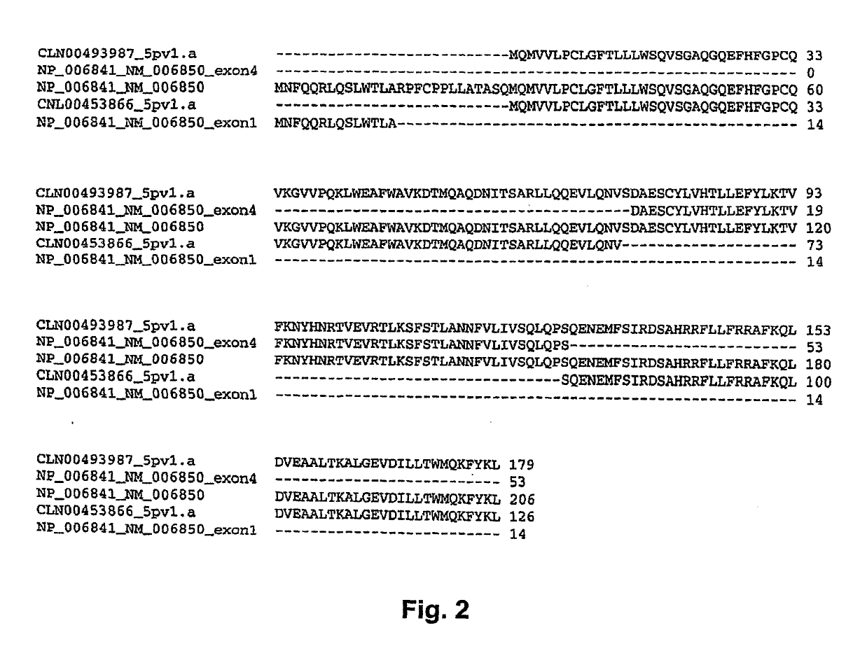

Table 1 provides information regarding the sequences of the Sequence Listing. Column 1 shows an internal designation identification number (FP ID); column 2 shows the nucleotide sequence ID number for the open reading frame of the nucleic acid sequence (SEQ. ID. NO. (N1)); column 3 shows the amino acid sequence identification number for the polypeptide sequence (SEQ. ID. NO. (P1)); column 4 shows the nucleotide sequence identification number for the entire nucleic acid sequence (SEQ ID NO. (N0)) that may contain 5′ or 3′ UTR; column 5 shows the polypeptide identification number of the source of the clone or NCBI accession number (Source ID); and column 6 shows the classification/gene family or annotation of the sequence (Type).

Table 2 characterizes the splice variants or fragments of the invention. Column 1 shows an internal designation identification number of the polypeptide (FP ID); column 2 shows the clone identification number of the polypeptide (Clone ID); column 3 shows the predicted length of the polypeptide in number of amino acid residues (Pred Prot Len); column 4 shows the public accession identification number of a top human hit found in the National Center for Biotechnology Information (NCBI) public database, NR (Top Human Hit Access ID); column 5 shows the annotation of the top human hit set forth in column 4 (Top Human Hit Annotation); column 6 shows the length of the top human hit in number of amino acid residues (Top Human Hit Len); column 7 shows the length of the match in number of amino acid residues between the query sequence designated by the FP ID and the top human hit (Match Len); column 8 shows the percent identity between the FP ID and the top human hit over the length of the FP ID amino acid sequence expressed as a percentage (Top Human Hit % ID over Query Len); and column 9 shows the percent identity between the FP ID and the top human hit over the length of the top human hit (% ID over Hum Hit Len).

Table 3 shows amino acid coordinates of the splice variants: column 1 shows an internal designation ID number of the polypeptide (FP ID); column 2 shows the source ID number or NCBI accession number of the polypeptide (Clone ID); column 3 shows an internal cluster ID number of the polypeptide (Cluster); column 4 (Class) shows the classification of the polypeptide as secreted (SEC), single transmembrane (STM), and an indication of whether it is a type I or type II STM; column 5 shows the predicted protein length in number of amino acid residues (Pred Prot Len); column 6 shows an internal parameter predicting the likelihood that the FP ID is secreted (Treevote), with “1” being a high likelihood of the polypeptide being secreted and “0” being a low likelihood of being secreted; column 7 shows the protein coordinates of the mature polypeptide with first amino acid residue at the N-terminus of the full-length polypeptide being amino acid number 1 (Mat Prot Coords); column 8 shows an alternate prediction of the mature protein coordinates (Alt Mat Prot Coords); column 9 shows the signal peptide coordinates, if any (Sig Pep Coords); column 10 shows the number of transmembrane domains (TM) present in the polypeptide; column 11 shows the coordinates of transmembrane domains (TM Coords); column 12 shows the coordinates of non-transmembrane domains (Non-TM Coords); column 13 shows the names of pfam domains within the polypeptide (Pfam).

Table 4 shows the coordinates of the predicted pfam domains in the APO2L polypeptides: column 1 shows an internal designation ID number for the polypeptide (FP ID); column 2 shows the source ID number or NCBI accession number for the polypeptide (Source ID); column 3 shows the name of the pfam domain (Pfam); column 4 shows the coordinates of the beginning and ending amino acid residues spanning the pfam domain in the polypeptide (Coords).

Table 5 characterizes the leader sequences for production of the secreted proteins of the invention. Column 1 shows an internal designation ID number of the polypeptide (FP ID); column 2 shows the source of the leader sequence as specified by the name of the protein or NCBI accession number and the beginning and ending amino acid coordinates of the leader sequence (Source ID); and column 3 provides an annotation identifying each leader sequence (Annotation).

### DETAILED DESCRIPTION OF THE INVENTION

### Definitions

The terms used herein have their ordinary meaning and the meanings given them specifically below.

The terms “nucleic acid molecule,” “nucleotide,” “polynucleotide,” and “nucleic acid” are used interchangeably herein to refer to polymeric forms of nucleotides of any length. They can include both double- and single-stranded sequences and include, but are not limited to, cDNA from viral, prokaryotic, and eucaryotic sources; mRNA; genomic DNA sequences from viral (e.g. DNA viruses and retroviruses) or prokaryotic sources, RNAi, cRNA, anti-sense molecules, ribozymesand synthetic DNA sequences. The term also captures sequences that include any of the known base analogs of DNA and RNA.

“Recombinant,” as used herein to describe a nucleic acid molecule means a polynucleotide of genomic, cDNA, viral, semisynthetic, and/or synthetic origin which, by virtue of its origin or manipulation, is not associated with all or a portion of the polynucleotide with which it is associated in nature. The term “recombinant” as used with respect to a protein or polypeptide, means a polypeptide produced by expression of a recombinant polynucleotide. The term “recombinant” as used with respect to a host cell means a host cell into which a recombinant polynucleotide has been introduced.

A “complement” of a nucleic acid molecule is a one that is comprised of its complementary base pairs. Deoxyribonucleotides with the base adenine are complementary to those with the base thymidine, and deoxyribonucleotides with the base thymidine are complementary to those with the base adenine. Deoxyribonucleotides with the base cytosine are complementary to those with the base guanine, and deoxyribonucleotides with the base guanine are complementary to those with the base cytosine. Ribonucleotides with the base adenine are complementary to those with the base uracil, and deoxyribonucleotides with the base uracil are complementary to those with the base adenine. Ribonucleotides with the base cytosine are complementary to those with the base guanine, and deoxyribonucleotides with the base guanine are complementary to those with the base cytosine.

A “promoter,” as used herein, is a DNA regulatory region capable of binding RNA polymerase in a mammalian cell and initiating transcription of a downstream (3′ direction) coding sequence operably linked thereto. For purposes of the present invention, a promoter sequence includes the minimum number of bases or elements necessary to initiate transcription of a gene of interest at levels detectable above background. Within the promoter sequence is a transcription initiation site, as well as protein binding domains (consensus sequences) responsible for the binding of RNA polymerase. Eucaryotic promoters will often, but not always, contain “TATA” boxes and “CAT” boxes. Promoters include those that are naturally contiguous to a nucleic acid molecule and those that are not naturally contiguous to a nucleic acid molecule. Additionally, a promoter includes inducible promoters, conditionally active promoters, such as a cre-lox promoter, constitutive promoters, and tissue specific promoters.

A “vector” is a plasmid that can be used to transfer DNA sequences from one organism to another or to express a gene of interest.

The term “host cell” includes an individual cell, cell line, cell culture, or cell in vivo, which can be or has been a recipient of any polynucleotides or polypeptides of the invention, for example, a recombinant vector, an isolated polynucleotide, an antibody or a fusion protein. Host cells include progeny of a single host cell, and the progeny may not necessarily be completely identical (in morphology, physiology, or in total DNA, RNA, or polypeptide complement) to the original parent cell due to natural, accidental, or deliberate mutation and/or change. Host cells can be prokaryotic or eukaryotic, including mammalian, insect, amphibian, reptilian, crustacean, avian, fish, plant, and fungal cells. A host cell includes cells transformed, transfected, transduced, or infected in vivo or in vitro with a polynucleotide of the invention, for example, a recombinant vector. A host cell which comprises a recombinant vector of the invention may be called a “recombinant host cell.”

“Operably linked” refers to an arrangement of elements wherein the components so described are configured so as to perform their desired function. Thus, a given promoter operably linked to a coding sequence is capable of effecting the expression of the coding sequence when the proper transcription factors, etc., are present. The promoter need not be contiguous with the coding sequence, so long as it functions to direct the expression thereof. Thus, for example, intervening untranslated yet transcribed sequences can be present between the promoter sequence and the coding sequence, as can translated introns, and the promoter sequence can still be considered “operably linked” to the coding sequence.

The terms “polypeptide” and “protein” are used interchangeably to refer to a polymer of amino acid residues, and are not limited to a minimum length. Thus, peptides, oligopeptides, dimers, multimers, and the like, are included within the definition. Both full-length proteins and fragments thereof are encompassed by the definition. The terms also include post-expression modifications of the polypeptide, for example, glycosylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

By “isolated” is meant, when referring to a polynucleotide or polypeptide of the invention, that the indicated molecule is substantially separated, e.g., from the whole organism in which the molecule is found or from the cell culture in which the antibody is produced, or is present in the substantial absence of other biological macromolecules of the same type. For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically.

“Secretory leader,” “signal peptide,” or a “leader sequence,” are used interchangeably herein to refer to a sequence of amino acid residues, typically positioned at the N terminus of a polypeptide, which directs the intracellular trafficking of a polypeptide. Polypeptides that contain a secretory leader, signal peptide, or leader sequence typically also contain a secretory leader, signal peptide, or leader sequence cleavage site. Such polypeptides, after cleavage at the cleavage sites, generate mature polypeptides, for example, after extracellular secretion or after being directed to an appropriate intracellular compartment.

By “fragment” is intended a polypeptide consisting of only a part of the intact full-length or naturally occurring polypeptide sequence and structure. The fragment can include e.g., a C-terminal deletion, an N-terminal deletion, and/or an internal deletion of a native polypeptide or an extracellular domain of a transmembrane protein. A fragment of a protein will generally include at least about 5-10, 15-25, or 20-50 or more contiguous amino acid residues of the full-length molecule, at least about 15-25 contiguous amino acid residues of the full-length molecule, or any integer between 5 amino acids and the full-length sequence.

A “biologically active” entity, or an entity having “biological activity,” is one having structural, regulatory, or biochemical functions of a naturally occurring molecule or any function related to or associated with a metabolic or physiological process. Biologically active polynucleotide fragments are those exhibiting activity similar, but not necessarily identical, to an activity of a polynucleotide of the present invention. The biological activity can include an improved desired activity, or a decreased undesirable activity. For example, an entity demonstrates biological activity when it participates in a molecular interaction with another molecule, such as hybridization, when it has therapeutic value in alleviating a disease condition, when it has prophylactic value in inducing an immune response, when it has diagnostic value in determining the presence of a molecule, such as a biologically active fragment of a polynucleotide that can, e.g., be detected as unique for the polynucleotide molecule, or that can be used as a primer in PCR. A biologically active polypeptide or fragment thereof includes one that can participate in a biological reaction, for example, one that can serve as an epitope or immunogen to stimulate an immune response, such as production of antibodies, or that can participate in signal transduction by binding to receptors, proteins, or nucleic acids, or activating enzymes or substrates.

“Expression of a nucleic acid molecule” refers to the conversion of the information contained in the molecule, into a gene product. A gene product can be the direct transcriptional product of a gene (e.g., mRNA, tRNA, rRNA, antisense RNA, ribozyme, structural RNA, or any other type of RNA) or a peptide or polypeptide produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

A “fusion partner” is a polypeptide fused in-frame at the N-terminus and/or C-terminus of a therapeutic or prophylactic polypeptide, or internally to a therapeutic or prophylactic polypeptide.

The term “receptor” refers to a polypeptide that binds to a specific extracellular molecule and may initiate a cellular response.

“Death domain receptors” are receptors that comprise one or more death domain, which is a sequence, typically located in the cytosolic portion of a transmembrane receptor, involved in TNF-mediated cell signaling. Death domain receptors may have one or more of many known functions, including regulating apoptosis.

The term “antibody” or “immunoglobulin” refers to a protein, e.g., one generated by the immune system, synthetically, or recombinantly, that is capable of recognizing and binding to a specific antigen; antibodies are commonly known in the art. The term includes active fragments, including for example, an antigen binding fragment of an immunoglobulin, a variable and/or constant region of a heavy chain, a variable and/or constant region of a light chain, a complementarity determining region (cdr), and a framework region. The terms include polyclonal and monoclonal antibody preparations, as well as preparations including hybrid antibodies, altered antibodies, chimeric antibodies, hybrid (chimeric) antibody molecules, F(ab′)₂ and F(ab) fragments; Fv molecules (e.g., noncovalent heterodimers), dimeric and trimeric antibody fragment constructs; minibodies, humanized antibody molecules and any functional fragments obtained from such molecules, wherein such fragments retain specific binding.

An “agent,” e.g., an anti-cancer agent, describes any substance, whether synthetic or semi-synthetic; natural, organic, or inorganic; a small molecule or a macromolecule; a pharmaceutical or a protein, with the capability of altering a biological activity. The biological activity can be measured using any assay known in the art.

As used herein, the phrase “pharmaceutically acceptable carrier” is intended to include substances that can be co-administered with the compositions of the invention that allow the composition or active molecule therein to perform its intended function. Examples of such carriers include solutions, solvents, buffers, adjuvants, dispersion media, delay agents, emulsions, and the like. Further, any other conventional carrier, suitable for use with the described compositions, fall within the scope of the instant invention, such as, for example, phosphate buffered saline.

The terms “subject,” “individual,” “host,” and “patient” are used interchangeably herein to refer to a living animal, including a human and a non-human animal. The subject may, e.g., be an organism possessing immune cells capable of responding to antigenic stimulation, and stimulatory and inhibitory signaling transduction through cell surface receptor binding. The subject may be a mammal, such as a human or nonhuman mammal, for example, dogs, cats, pigs, cows, sheep, goats, horses, rats, and mice. The term “subject” does not preclude individuals that are entirely normal with respect to a disease, or normal in all respects.

“Treatment,” as used herein, covers any treatment of a condition or disease in a mammal, including a human, and includes preventing the condition or disease from occurring or recurring in a subject who may be predisposed to the condition or disease but has not yet been diagnosed; inhibiting the condition or disease, i.e., arresting its development; relieving the condition or disease, i.e., causing regression of the condition or disease; restoring or repairing a lost, missing, or defective function; or stimulating an inefficient process. In the context of cancer, the term “treating” includes preventing growth of tumor cells or cancer cells, preventing replication of tumor cells or cancer cells, lessening the overall tumor burden, and ameliorating one or more symptoms associated with the disease.

A “disease” is a pathological condition, e.g., one that can be identified by symptoms or other identifying factors as diverging from a healthy or a normal state. The term “disease” includes disorders, syndromes, conditions, and injuries. Diseases include, but are not limited to, proliferative, inflammatory, immune, metabolic, infectious, and ischemic diseases.

A “modulator” of the polypeptides or polynucleotides or an “agent” herein is an agonist or antagonist that interferes with the binding or activity of such polypeptides or polynucleotides. Such modulators or agents include, for example, polypeptide variants, whether agonist or antagonist; antibodies, whether agonist or antagonist; soluble receptors, usually antagonists; small molecule drugs, whether agonist or antagonist; RNAi, usually an antagonist; antisense molecules, usually an antagonist; and ribozymes, usually an antagonist. In some embodiments, an agent is a subject polypeptide, where the subject polypeptide itself is administered to an individual. In some embodiments, an agent is an antibody specific for a subject “target” polypeptide. In some embodiments, an agent is a chemical compound such as a small molecule that may be useful as an orally available drug. Such modulation includes the recruitment of other molecules that directly effect the modulation. For example, an antibody that modulates the activity of a subject polypeptide that is a receptor on a cell surface may bind to the receptor and fix complement, activating the complement cascade and resulting in lysis of the cell. An agent which modulates a biological activity of a subject polypeptide or polynucleotide increases or decreases the activity or binding at least about 10%, at least about 15%, at least about 20%, at least about 25%, at least about 50%, at least about 80%, or at least about 2-fold, at least about 5-fold, or at least about 10-fold or more when compared to a suitable control.

### Nucleic Acids and Polypeptides

The present invention provides nucleic acid molecules containing a polynucleotide encoding a newly identified APO2L variant polypeptide and two newly identified IL-24 variant polypeptides having the amino acid sequences as shown in the Sequence Listing. The isolated APO2L and IL-24 variants of the invention were identified by bioinformatic analysis. The APO2L variant polypeptide is structurally related to members of the tumor necrosis factor (TNF) gene superfamily, and has an open reading frame encoding a polypeptide of 235 amino acids (SEQ ID NO.: 15). The IL-24 variant polypeptides are related to members of the IL-10 cytokine family. The first IL-24 variant contains an open reading frame encoding a polypeptide of 179 amino acids (SEQ ID NO.:6), and the second IL-24 variant contains an open reading frame encoding a polypeptide of 126 amino acids (SEQ ID NO.:8). IL-24 has been found to be predominantly expressed in, for example, the trachea, bile duct, cerebral cortex, smooth muscle, joint meniscus, brain, and tonsil.

Fragments of the full length APO2L and IL-24 variants may be used as hybridization probes for cDNA libraries to isolate the full length gene and to isolate other genes which have a high sequence similarity or a similar biological activity. Probes of this type can have at least 30 bases and may comprise, for example, 50 or more bases. The probe may also be used in a screening procedure to identify a cDNA clone corresponding to a full length transcript and a genomic clone or clones that contain the complete APO2L and IL-24 genes, including regulatory and promoter regions, exons, and introns. An example of such a screen would include isolating the coding regions of the APO2L and IL-24 genes by using a known nucleic acid sequence to synthesize an oligonucleotide probe. Labeled oligonucleotides having a sequence complementary to a gene of the present invention can be used to screen a human cDNA, a genomic DNA, or a mRNA library to identify complementary library components.

The present invention further relates to polynucleotides which hybridize to the hereinabove-described sequences if there is at least 91%, at least 92%, or at least 95% identity between the sequences. The present invention relates to polynucleotides which hybridize under stringent conditions to the hereinabove-described polynucleotides. Stringent conditions generally include condition under which hybridization will occur only if there is at least 95%, or at least 97% identity between the sequences. For example, overnight incubation at 42° C. in a solution containing 50% formamide, 5×SSC (150 mM NaCl, 15 mM trisodiurn citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65° C., constitute stringent conditions.

The polynucleotides which hybridize to the hereinabove described polynucleotides in a preferred embodiment encode polypeptides which either retain substantially the same biological function or activity as the mature polypeptide.

Alternatively, the polynucleotide may have at least 20 bases, at least 30 bases, or at least 50 bases which hybridize to a polynucleotide of the present invention and which has an identity thereto, as hereinabove described, and which may or may not retain activity.

Thus, the present invention is directed to polynucleotides having at least a 70% identity, at least a 90% identity, or at least a 95% identity to a polynucleotide which encodes the polypeptides set forth in the Sequence Listing, as well as fragments thereof, which fragments have at least 30 bases or at least 50 bases, and to polypeptides encoded by such polynucleotides.

Using the information provided herein, such as the nucleotide sequences set forth in the Sequence Listing, nucleic acid molecules of the present invention encoding an APO2L and IL-24 polypeptide may be obtained using standard cloning and screening procedures, such as those for cloning cDNAs using mRNA as starting material. Nucleic acids of the invention are useful as hybridization probes for differential identification of the tissue(s) or cell type(s) present in a biological sample. Polypeptides and antibodies directed to those polypeptides are useful for providing immunological probes for the differential identification of tissues or cell types.

Variant and Mutant Polynucleotides

The present invention further relates to variants of the nucleic acid molecules of the present invention, which encode portions, analogs, or derivatives of the APO2L and IL-24 molecules. Variants may occur naturally, such as a natural allelic variant, i.e., one of several alternate forms of a gene occupying a given chromosomal locus Genes II; Lewin, B., ed., John Wiley & Sons, New York (1985)). Non-naturally occurring variants may be produced using art-known mutagenesis techniques.

Such variants include those produced by nucleotide substitutions, deletions, or additions. The substitutions, deletions, or additions may involve one or more nucleotides. The variants may be altered in coding regions, non-coding regions, or both. Alterations in the coding regions may produce conservative or non-conservative amino acid substitutions, deletions or additions. These may take the form of silent substitutions, additions, or deletions which do not alter the properties or activities of the described APO2L and IL-24 proteins, or portions thereof.

In an embodiment, the invention provides nucleic acid molecules encoding mature proteins, i.e., those with cleaved signal peptide or leader sequences, e.g., as shown in the Sequence Listing. Further embodiments include an isolated nucleic acid molecule comprising a polynucleotide having a nucleotide sequence at least 93% identical, or at least 95%, 96%, 97%, 98% or 99% identical to a polynucleotide from the Sequence Listing, a polypeptide encoded by a polynucleotide shown in the Sequence Listing, a polypeptide shown in the Sequence Listing, or a biologically active fragment of any of these.

A polynucleotide having a nucleotide sequence at least, for example, 95% identical to a reference nucleotide sequence encoding an APO2L or IL-24 polypeptide is one in which the nucleotide sequence is identical to the reference sequence except that it may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 93%, 95%, 96%, 97%, 98%, or 99% identical to, for instance, the nucleotide sequences set forth in the Sequence Listing can be determined conventionally using known computer programs such as the Bestfit prograrn (Wisconsin Sequence Analysis Package, Version 8 for Unix, Genetics Computer Group, Madison, Wis.). Bestfit uses the local homology algorithm of Smith and Waterman, Advanices in Applied Mathematics 2:482-489 (1981), to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed.

The present application is directed to nucleic acid molecules at least 93%, 95%, 96%, 97%, 98%, or 99% identical to the nucleic acid sequences set forth in the Sequence Listing irrespective of whether they encode a polypeptide having APO2L or IL-24 activity. Even where a particular nucleic acid molecule does not encode a polypeptide having APO2L or IL-24 activity, one of skill in the art would know how to use the nucleic acid molecule, for instance, as a hybridization probe or a polymerase chain reaction (PCR) primer. Uses of the nucleic acid molecules of the present invention that do not encode a polypeptide having APO2L or IL-24 activity include, inter alia, (1) isolating the APO2L or IL-24 gene or allelic variants thereof in a cDNA library; (2) in situ hybridization (e.g., “FISH”) to metaphase chromosomal spreads to provide the precise chromosomal location of the APO2L or IL-24 genes, as described in Verna et al., Human Chromosomes: A Manual of Basic Techniques, Pergamon Press, New York (1988); and Northern blot analysis for detecting APO2L or IL-24 mRNA expression in specific tissues.

The present application is also directed to nucleic acid molecules having sequences at least 93%, 95%, 96%, 97%, 98%, or 99% identical to a nucleic acid sequence of the Sequence Listing which, encode a polypeptide having APO2L or IL-24 polypeptide activity, i.e., a polypeptide exhibiting activity either identical to or similar, but not identical, to an activity of the APO2L and IL-24 polypeptides of the invention, as measured in a particular biological assay. For example, the APO2L and IL-24 polypeptides of the present invention may either stimulate or inhibit the proliferation of various mammalian cells, as demonstrated below.

Of course, due to the degeneracy of the genetic code, one of ordinary skill in the art will immediately recognize that a large number of the nucleic acid molecules having a sequence at least 93%, 95%, 96%, 97%, 98%, or 99% identical to the nucleic acid sequence of the nucleic acid sequences set forth in the Sequence Listing will encode a polypeptide having APO2L or IL-24 polypeptide activity. In fact, since multiple degenerate variants of these nucleotide sequences encode the same polypeptide, this will be clear to the skilled artisan even without performing the above described comparison assay. It will be further recognized in the art that a reasonable number of nucleic acid molecules that are not degenerate variants will also encode a polypeptide having APO2L or IL-24 polypeptide activity, the skilled artisan is filly aware of amino acid substitutions that are either less likely or not likely to significantly affect protein function (e.g., replacing one aliphatic amino acid with a second aliphatic amino acid), as further described below.

Vectors and Host Cells

The present invention also relates to vectors which include the isolated nucleic acid molecules of the present invention, host cells which are genetically engineered with the recombinant vectors, and the production of APO2L and IL-24 polypeptides or fragments thereof by recombinant techniques. The vector may be, for example, a phage, plasmid, viral, or retroviral vector. Retroviral vectors may be replication competent or replication defective. In the latter case, viral propagation generally will occur only in complementing host cells.

The present invention provides recombinant vectors that contain, for example, nucleic acid constructs that encode secretory leader sequences and a selected heterologous polypeptide of interest, and host cells that are genetically engineered with the recombinant vectors. Selected heterologous polypeptides of interest in the present invention include, for example, an extracellular fragment of a secreted protein, a type I membrane protein, a type II membrane protein, a multi-membrane protein, and a soluble receptor. These vectors and host cells can be used for the production of polypeptides described herein, including fragments thereof by conventional recombinant techniques. The vector may be, for example, a phage, plasmid, viral or retroviral vector. Retroviral vectors may be replication competent or replication defective. As above, in the latter case, viral propagation generally will occur only in complementing host cells.

The polynucleotides may be joined to a vector containing a secretory leader sequence (see, for example, the Sequence Listing), and a selectable marker for propagation in a host. Generally, a plasmid vector is introduced in a precipitate, such as a calcium phosphate precipitate, or in a complex with a charged lipid. If the vector is a virus, it may be packaged iii vitro using an appropriate packaging cell line and then transduced into host cells.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged ill vitro using an appropriate packaging cell line and then transduced into host cells.

The DNA insert can be operatively linked to an appropriate promoter, such as the phage lambda PL promoter; the E. coli lac, trp, phoA and tac promoters; the SV40 early and late promoters; and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs can include a translation initiating codon at the beginning and a termination codon (UAA, UGA, or UAG) appropriately positioned at the end of the polypeptide to be translated.

As indicated, the expression vectors may include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera S f9 cells; animal cells such as CHO, COS, 293 and Bowes melanoma cells; and plant cells. Appropriate culture mediums and conditions for the above-described host cells are known in the art.

The selectable markers are genes that confer a phenotype on a cell expressing the marker, so that the cell can be identified under appropriate conditions. Generally, a selectable marker allows the selection of transformed cells based on their ability to thrive in the presence or absence of a chemical or other agent that inhibits an essential cell function. Suitable markers, therefore, include genes coding for proteins which confer drug resistance or sensitivity thereto, impart color to, or change the antigenic characteristics of those cells transfected with a molecule encoding the selectable marker, when the cells are grown in an appropriate selective medium. For example, selectable markers include cytotoxic markers and drug resistance markers, whereby cells are selected by their ability to grow on media containing one or more of the cytotoxins or drugs; auxotrophic markers by which cells are selected for their ability to grow on defined media with or without particular nutrients or supplements, such as thymidine and hypoxanthine; metabolic markers for which cells are selected, e.g., their ability to grow on defined media containing the appropriate sugar as the sole carbon source, and markers which confer the ability of cells to form colored colonies on chromogenic substrates or cause cells to fluoresce.

Among vectors suitable for use in bacteria include pQE70, pQE60, and pQE-9, available from QIAGEN, Inc., supra; pBS vectors, Phagescript vectors, Bluescript vectors, pNH8A, pNH6a, pNH18A, pNH46A, available from Stratagene (La Jolla, Calif.); and ptrc99a, pKK223-3, pKK233-3, pDR540, pRIT5 available from Pharmacia (Peapack, N.J.). Among suitable eukaryotic vectors are pWLNEO, pSV2CAT, pOG44, pXT1, and pSG available from Stratagene; and pSVK3, pBPV, pMSG and pSVL, available from Pharmacia. Other suitable vectors will be readily apparent to the skilled artisan.

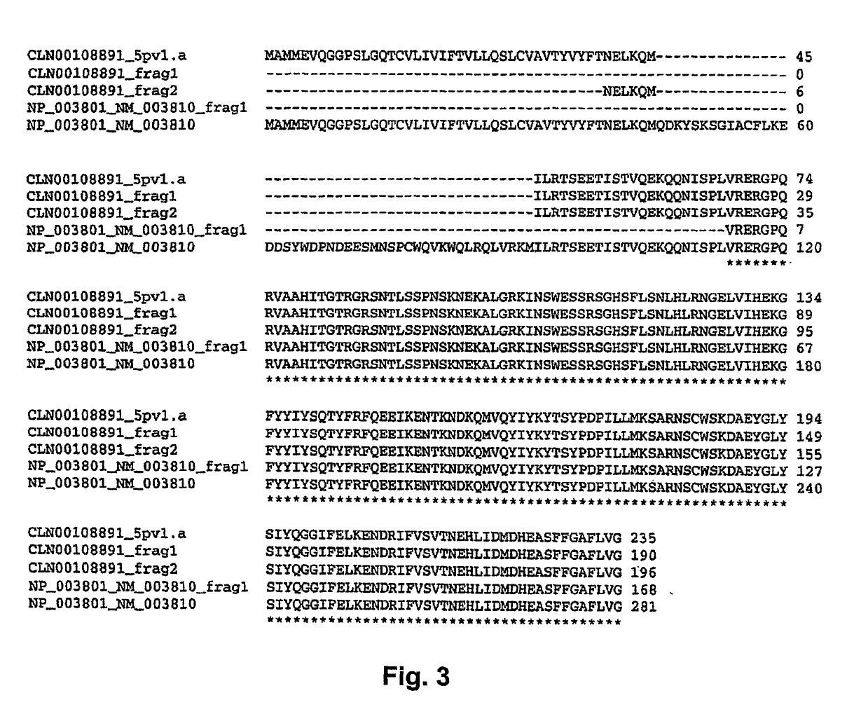

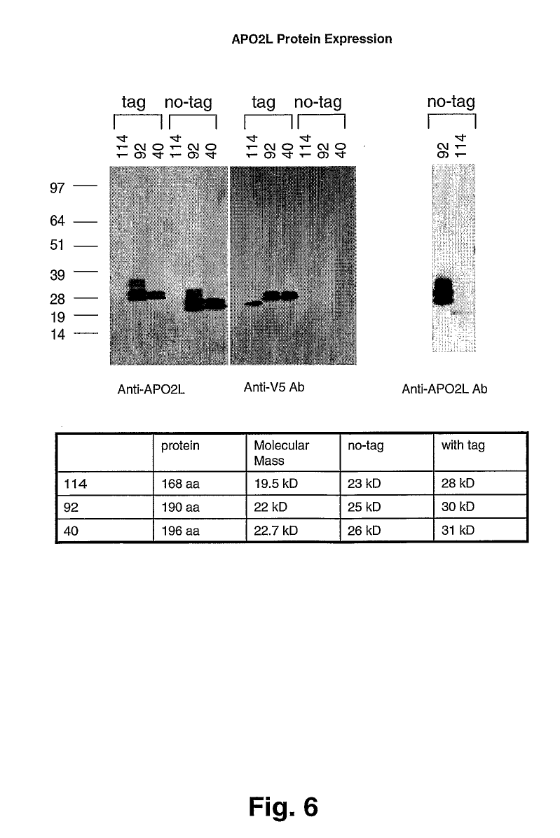

Other suitable vectors include those employing a pTT vector backbone, see, for example, FIGS. 3-7 (Durocher et al. Nucl. Acids Res. 30 (2002)). Briefly, the pTT vector backbone may be prepared by obtaining pIRESpuro/EGFP (pEGFP) and pSEAP basic vector(s), for example from Clontech (Palo Alto, Calif.), and pcDNA3.1, pCDNA3.1/Myc-(His)₆ and pCEP4 vectors can be obtained from, for example, Invitrogen. SuperGlo GFP variant (sgGFP) can be obtained from Q-Biogene (Carlsbad, Calif.). Preparing a pCEP5 vector can be accomplished by removing the CMV proinoter and polyadenylation signal of pCEP4 by sequential digestion and self-ligation using SalI and XbaI enzymes resulting in plasmid pCEP4Δ. A GblII fragment from pAdCMV5 (Massie et al., J Virol., 72: 2289-2296 (1998)), encoding the CMV5-poly(A) expression cassette ligated in BglII-linearized pCEP4Δ, resulting in pCEP5 vector. The pTT vector can be prepared by deleting the hygromycin (BsmnI and SalI excision followed by fill-in and ligation) and EBNA1 (ClaI and NsiI excision followed by fill-in and ligation) expression cassettes. The ColEI origin (FspI-SalI fragment, including the 3′ end of β-lactamase ORF) can be replaced with a FspI-SalI fragment from pcDNA3.1 containing the pMBI origin (and the same 3+ end of β-lactamase ORF). A Myc-(His)₆ C-terminal fusion tag can be added to SEAP (HindIII-HpaI fragment from pSEAP-basic) following in-frame ligation in pcDNA3.1/Myc-His digested with HindII and EcoRV. Plasmids can subsequently be amplified in Escherichia coli (E. coli) (DH5α) grown in LB medium and purified using MAXI prep columns (Qiagen, Mississauga, Ontario, Canada). To quantify, plasmids can be subsequently diluted in 50 mM Tris-HCl pH 7.4 and absorbencies can be measured at 260 nm and 280 nm. Plasmid preparations with A₂₆₀/A₂₈₀ ratios between about 1.75 and about 2.00 are suitable.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986).

The polypeptides may be expressed in a modified form, such as a fusion protein, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties may be added to the polypeptide to facilitate purification. Such regions may be removed prior to final preparation of the polypeptide.

The addition of peptide moieties to polypeptides to engender secretion or excretion, to improve stability and to facilitate purification, among others, are familiar and routine techniques in the art. A suitable fusion protein may comprise a heterologous region from immunoglobulin that is useful to stabilize and purify proteins. For example, EP-A-O 464 533 (Canadian counterpart 2045869) discloses fusion proteins containing various portions of constant region of immunoglobulin molecules together with another human protein or part thereof. In many cases, the Fc part in a fusion protein is thoroughly advantageous for use in therapy and diagnosis and thus results, for example, in improved pharmacokinetic properties (EP-A 0232 262). On the other hand, for some uses it would be desirable to be able to delete the Fc part after the fusion protein has been expressed, detected, and purified in the advantageous manner described. This is the case when the Fc portion proves to be a hindrance to use in therapy and diagnosis, for example when the fusion protein is to be used as an antigen for immunizations. In drug discovery, for example, human proteins, such as hIL-5, have been fused with Fc portions for the purpose of high-throughput screening assays to identify antagonists. See, Bennett et al., J. Molec. Recog., 8:52-58 (1995) and Johanson et al, J. Biol. Chem., 270:9459-9471 (1995).

The APO2L and IL-24 polypeptides can be recovered and purified from recombinant cell cultures by well-known methods, including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. High performance liquid chromatography (HPLC) can be employed for purification. Polypeptides of the present invention include products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes. Thus, it is well known in the art that the N-terminal methionine encoded by the translation initiation codon generally is removed with high efficiency from any protein after translation in eukaryotic cells. While the N-terminal methionine on most proteins also is efficiently removed in most prokaryotes, for some proteins this prokaryotic removal process is inefficient, depending on the nature of the amino acid to which the N-terminal methionine is covalently linked.

Typically, a heterologous polypeptide, whether modified or unmodified, may be expressed as described above, or as a fusion protein, and may include not only secretion signals, but also a secretory leader sequence. A secretory leader sequence of the invention directs certain proteins to the endoplasmic reticulum (ER). The ER separates the membrane-bound proteins from other proteins. Once localized to the ER, proteins can be further directed to the Golgi apparatus for distribution to vesicles; including secretory vesicles; the plasma membrane, lysosomes, and other organelles.

Proteins targeted to the ER by a secretory leader sequence can be released into the extracellular space as a secreted protein. For example, vesicles containing secreted proteins can fuse with the cell membrane and release their contents into the extracellular space—a process called exocytosis. Exocytosis can occur constitutively or after receipt of a triggering signal. In the latter case, the proteins may be stored in secretory vesicles (or secretory granules) until exocytosis is triggered. Similarly, proteins residing on the cell membrane can also be secreted into the extracellular space by proteolytic cleavage of a “linker” holding the protein to the membrane.

Additionally, peptide moieties and/or purification tags may be added to the polypeptide to facilitate purification. Such regions may be removed prior to final preparation of the polypeptide. The addition of peptide moieties to polypeptides to engender secretion or excretion, to improve stability, and to facilitate purification, among other reasons, are familiar and routine techniques in the art. Suitable purification tags include, for example, V5, HISX6, HISX8, avidin, and biotin.

The invention provides a fusion protein comprising a heterologous region from an immunoglobulin that is useful to stabilize and purify proteins. For example, EP-A-O 464 533 (Canadian counterpart 2045869) discloses fusion proteins containing various portions of constant region of immunoglobulin molecules together with another human protein or part thereof. In many cases, the Fc part of a fusion protein is advantageous for use in therapy and diagnosis and thus results, for example, in improved pharmacokinetic properties (EP-A 0232 262). On the other hand, for some uses it would be desirable to be able to delete the Fc part after the fusion protein has been expressed, detected, and purified in the advantageous manner described. This is the case when the Fc portion proves to be a hindrance to use in therapy and/or diagnosis, for example, when the fusion protein is to be used as an antigen for immunizations. In drug discovery, for example, human proteins, such as hIL-5, have been fused with Fc portions for the purpose of high-throughput screening assays to identify antagonists of hIL-5. See, Bennett et al., J. Molec. Recog., 8:52-58 (1995) and Johanson et al, J. Biol. Chem., 270:9459-9471 (1995).

A heterologous polypeptide of the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, lectin chromatography, and high performance liquid chromatography (HPLC). Polypeptides of the present invention include products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells, or from a cell free expression system. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins this prokaryotic removal process is inefficient, depending on the nature of the amino acid to which the N-terminal methionine is covalently linked.

Polypeptides and Fragments

The invention further provides isolated APO2L and IL-24 polypeptides containing the amino acid sequences encoded by the nucleotide sequences set forth in the Sequence Listing, the amino acid sequences set forth in the Sequence Listing, and polypeptides comprising a fragment of any of these.

The invention provides secreted proteins, which are capable of being directed to the endoplasmic reticulum (ER), secretory vesicles, or the extracellular space as a result of a secretory leader, signal peptide, or leader sequence, as well as proteins released into the extracellular space without necessarily containing a signal sequence. If a secreted protein is released into the extracellular space, it may undergo extracellular processing to a mature polypeptide. Release into the extracellular space can occur by many mechanisms, including exocytosis and proteolytic cleavage.

The sequences of the invention encompass a variety of different types of nucleic acids and polypeptides with different structures and functions. They can encode or comprise polypeptides belonging to different protein families (Pfam). The “Pfam” system is an organization of protein sequence classification and analysis, based on conserved protein domains; it can be publicly accessed in a number of ways, for example, at http://pfam.wustl.edu. Protein domains are portions of proteins that have a tertiary structure and sometimes have enzymatic or binding activities; multiple domains can be connected by flexible polypeptide regions within a protein. Pfam domains can comprise the N-terminus or the C-terminus of a protein, or can be situated at any point in between. The Pfam system identifies protein families based on these domains and provides an annotated, searchable database that classifies proteins into families (Bateman et al. , Nucl. Acids Res. 30:276-280 (2002)). Sequences of the invention can encode or be comprised of more than one Pfam.

Sequences of the invention may comprise a tumor necrosis factor (TNF) pfam domain (http://pfam.wustl.edu/cgi-bin/getdesc?name=TNF), as further described below. TNF encompasses a family of receptor ligands that display pleiotropic effects on normal and malignant cells. Natural induction of TNF can be protective, but its overproduction may be detrimental and even lethal to the host. TNF has a wide variety of functions and elicits a variety of responses in different cell types. It was originally characterized as an antitumor agent and a cytotoxic factor for malignant cells. TNF subverts the electron transport system of mitochondria to produce oxygen radicals, which can kill malignant cells lacking protective enzymes. It can cause cytolysis of certain tumor cell lines; it is involved in the induction of cachexia; it is a potent pyrogen, causing fever by direct action or by stimulation of interleukin-1 secretion; and it can stimulate cell proliferation and induce cell differentiation under certain conditions. TNF also plays a role in the defense against viral, bacterial, and parasitic infections, and in mediating autoimmune responses (Fiers, FEBS Lett. 285:199-212 (1991)).

Variant and Mutant Polypeptides

Protein engineering may be employed to improve or alter the characteristics of APO2L and IL-24 polypeptides of the invention. Recombinant DNA technology known to those skilled in the art can be used to create novel mutant proteins or “muteins” including single or multiple amino acid substitutions, deletions, additions, or fusion proteins. Such modified polypeptides can show, e.g., enhanced activity or increased stability. In addition, they may be purified in higher yields and show better solubility than the corresponding natural polypeptide, at least under certain purification and storage conditions.

N-Terminal and C-Terminal Deletion Mutants

For instance, for many proteins, including the extracellular domain of a membrane associated protein or the mature form(s) of a secreted protein, it is known in the art that one or more amino acids may be deleted from the N-terminus or C-terminus without substantial loss of biological function. For instance, Ron et al., J. Biol. Chem., 268:2984-2988 (1993), reported modified KGF proteins that had heparin binding activity even if 3, 8, or 27 amino-terminal amino acid residues were missing.

However, even if deletion of one or more amino acids from the N-terminus of a protein results in modification or loss of one or more biological functions of the protein, other biological activities may still be retained. Thus, the ability of the shortened protein to induce and/or bind to antibodies which recognize the complete or mature from of the protein generally will be retained when less than the majority of the residues of the complete or mature protein are removed from the N-terminus. Whether a particular polypeptide lacking N-terminal residues of a complete protein retains such immunologic activities can readily be determined by routine methods described herein and otherwise known in the art. Accordingly, the present invention further provides polypeptides having one or more residues deleted from the amino terminus of the amino acid sequences of the APO2L and IL-24 molecules as shown in the Sequence Listing.

Similarly, many examples of biologically functional C-terminal deletion muteins are known. For instance, interferon gamma increases in activity as much as ten fold when 8-10 amino acid residues are deleted from the carboxy terminus of the protein, see, for example, Dobeli et al., J. Biotechnology, 7:199-216 (1988).

However, even if deletion of one or more amino acids from the C-terminus of a protein results in modification of loss of one or more biological functions of the protein, other biological activities may still be retained. Thus, the ability of the shortened protein to induce and/or bind to antibodies which recognize the complete or mature form of the protein generally will be retained when less than the majority of the residues of the complete or mature protein are removed from the C-terminus. Whether a particular polypeptide lacking C-terminal residues of a complete protein retains such immunologic activities can readily be determined by routine methods described herein and otherwise known in the art.

Other Mutants

In addition to terminal deletion forms of the protein discussed above, it also will be recognized by one of ordinary skill in the art that some amino acid sequences of the APO2L and IL-24 polypeptides can be varied without significant effect of the structure or function of the protein. If such differences in sequence are contemplated, it should be remembered that there will be critical areas on the protein which determine activity.

Thus, the invention further includes variations of the APO2L and IL-24 polypeptides which show substantial APO2L or IL-24 polypeptide activity or which include regions of the APO2L or IL-24 proteins such as the protein portions discussed below. Such mutants include deletions, insertions, inversions, repeats, and type substitutions, selected according to general rules known in the art, so as have little effect on activity. For example, guidance concerning how to make phenotypically silent amino acid substitutions is provided in Bowie et al., Science, 247:1306-1310 (1990), wherein the authors indicate that there are two main approaches for studying the tolerance of an amino acid sequence to change. The first method relies on the process of evolution, in which mutations are either accepted or rejected by natural selection. The second approach uses genetic engineering to introduce amino acid changes at specific positions of a cloned gene and selections, or screens, to identify sequences that maintain functionality.

As the authors state, these studies have revealed that proteins are surprisingly tolerant of amino acid substitutions. The authors further indicate which amino acid changes are likely to be permissive at a certain position of the protein. For example, most buried amino acid residues require nonpolar side chains, whereas few features of surface side chains are generally conserved. Other such phenotypically silent substitutions are described in Bowie, et al., supra, and the references cited therein. Typically seen as conservative substitutions are the replacements, one for another, among the aliphatic amino acids Ala, Val, Leu, and Ile; interchange of the hydroxyl residues Ser and Thr, exchange of the acidic residues Asp and Glu, substitution between the amide residues Asn and Gln, exchange of the basic residues Lys and Arg, and replacements between the aromatic residues Phe and Tyr.

Thus, a fragment, derivative, or analog of a polypeptide of the Sequence Listing or polypeptide encoded by a nucleic acid sequence of the Sequence Listing may be (i) one in which one or more of the amino acid residues are substituted with a conserved or non-conserved amino acid residue; such a substituted amino acid residue may or may not be one encoded by the genetic code; (ii) one in which one or more of the amino acid residues includes a substituent group; (iii) one in which the mature polypeptide is fused with another compound, such as a compound to increase the half-life of the polypeptide (for example, polyethylene glycol); or (iv) one in which the additional amino acids are fused to the above form of the polypeptide, such as an IgG Fc fusion region peptide, a leader or secretory sequence, a sequence employed to purify the above form of the polypeptide, or a proprotein sequence. Such fragments, derivatives, and analogs are deemed to be within the scope of those skilled in the art from the teachings herein.

Thus, the APO2L and IL-24 polypeptides of the present invention may include one or more amino acid substitutions, deletions, or additions, either from natural mutations or human manipulation. As indicated, these changes may be of a minor nature, such as conservative amino acid substitutions, that do not significantly affect the folding or activity of the protein. Conservative amino acid substitutions include the aromatic substitutions Phe, Trp, and Tyr; the hydrophobic substitutions Leu, Iso, and Val; the polar substitutions Glu and Asp; the basic substitutions Arg, Lys, and His; the acidic substitutions Asp and Glu; and the small amino acid substations Ala, Ser, Thr, Met, and Gly.

Amino acids essential for the functions of APO2L and IL-24 polypeptides can be identified by methods known in the art, such as site-directed mutagenesis or alanine-scanning mutagenesis, see, for example, Cunningham and Wells, Science, 244:1081-1085 (1989). The latter procedure introduces single alanine mutations. The resulting mutant molecules are then tested for biological activity such as receptor binding, or in vitro or in vitro proliferative activity.

Of special interest are substitutions of charged amino acids with other charged or neutral amino acids which may produce proteins with highly desirable improved characteristics, such as less aggregation. Aggregation may not only reduce activity but also be problematic when preparing pharmaceutical formulations, because, for example, aggregates can be immunogenic, Pinckard et al., Clin. Exp. Immunol., 2:331-340 (1967); Robbins et al., Diabetes, 36:838-845 (1987); Cleland et al., Crit. Rev. Therapeutic Drug Carrier Systems, 10:307-377 (1993).

Replacing amino acids can also change the selectivity of the binding of a ligand to cell surface receptors. For example, Ostade et al., Nature, 361:266-268 (1993) describes certain mutations resulting in selective binding of TNF-α to only one of the two known types of TNF receptors. Sites that are critical for ligand-receptor binding can also be determined by structural analysis such as crystallization, nuclear magnetic resonance, or photoaffinity labeling, for example, Smith et al., J. Mol. Biol., 224:899-904 (1992) and de Vos et al., Science, 255:306-312 (1992).

The polypeptides of the present invention can be provided in an isolated form, and can be substantially purified. A recombinantly produced version of the herein described APO2L and IL-24 polypeptides can be substantially purified, e.g., by the one-step method described in Smith and Johnson, Gene, 67:31-40 (1988). Polypeptides of the invention also can be purified from natural or recombinant sources using anti-APO2L and IL-24 antibodies of the invention using methods which are well known in the art of protein purification.

The polypeptides herein may be purified or isolated in the presence of ions or agents that aid in the refolding of the molecules or aid in dimerizing or trimerizing the molecules as conventional in the art. For example, Zn may be added to trimerize the APO2L fragments 40-45,92-281 or 92-281.

Further polypeptides of the present invention include polypeptides which have at least 93%, 95%, 96%, 97%, 98%, or 99% similarity to those described above. The polypeptides of the invention also contain those which are at least 93%, 94%, or 95%, 96%, 97%, 98%, or 99% identical to a polypeptide encoded by a nucleic acid sequence of the Sequence Listing.

The % similarity of two polypeptides can be measured by a similarity score determined by comparing the amino acid sequences of the two polypeptides using the Bestfit program with the default settings for determining similarity. Bestfit uses the local homology algorithm of Smith and Waterman, Advances in Applied Mathematics 2:482-489 (1981) to find the best segment of similarity between two sequences.

A polypeptide having an amino acid sequence at least, for example, 95% identical to a reference amino acid sequence of an APO2L or IL-24 polypeptide is one in which the amino acid sequence of the polypeptide is identical to the reference sequence except that the polypeptide sequence may include up to five amino acid alterations per each 100 amino acids of the reference polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids, up to 5% of the total amino acid residues in the reference sequence, may be inserted into the reference sequence. These alterations of the reference sequence may occur at the amino or carboxy terminal positions of the reference amino acid sequence or anywhere between those terminal positions, interspersed either individually among residues in the reference sequence, or in one or more contiguous groups within the reference sequence.

As a practical matter, whether any particular polypeptide is at least 93%, 95%, 96%, 97%, 98%, or 99% identical to, for instance, an amino acid sequence or to a polypeptide sequence encoded by a nucleic acid sequence set forth in the Sequence Listing can be determined conventionally using known computer programs, such the Bestfit program. When using Bestfit or other sequence alignment program to determine whether a particular sequence is, for instance, 95% identical to a reference sequence according to the present invention, the parameters are set, of course, that the percentage of identity is calculated over the fall length of the reference amino acid sequence and that gaps in homology of up to 5% of the total number of amino acid residues in the reference sequence are allowed.

As described in detail below, the polypeptides of the present invention can be used to raise polyclonal and monoclonal antibodies, which are useful in assays for detecting APO2L or IL-24 protein expression, also as described below, or as agonists and/or antagonists capable of enhancing or inhibiting APO2L or IL-24 protein function. These polypeptides can also be used in a yeast two-hybrid system to capture APO2L or IL-24 protein binding proteins, which are also candidate agonists and antagonists, according to the present invention. The yeast two hybrid system is described in Fields and Song, Nature, 340:245-246 (1989).

Epitope-Bearing Portions

In another aspect, the invention provides a polypeptide comprising an epitope-bearing portion of a polypeptide of the invention. The epitope of this polypeptide portion is an immunogenic or antigenic epitope of a polypeptide of the invention. Immunogenic epitopes are those parts of a protein that elicit an antibody response when the whole protein is provided as the immunogen. On the other hand, a region of a protein molecule to which an antibody can bind is an antigenic epitope. The number of immunogenic epitopes of a protein generally is less than the number of antigenic epitopes. See, for instance, Geysen et al., Proc. Natl. Acad. Sci., USA 81:3998-4002 (1983).

As to the selection of polypeptides bearing an antigenic epitope (i.e., that contain a region of a protein molecule to which an antibody can bind), it is well known in that art that relatively short synthetic peptides that mimic part of a protein sequence are routinely capable of eliciting an antiserum that reacts with the partially mimicked protein. See, for instance, Sutcliffe et al., Science, 219:660-666 (1983). Peptides capable of eliciting protein-reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals. Antigenic epitope-bearing peptides and polypeptides of the invention are therefore useful for raising antibodies, including monoclonal antibodies, that bind specifically to a polypeptide of the invention. See, for instance, Wilson et al., Cell, 37:767-778 (1984). The epitope-bearing peptides and polypeptides of the invention may be produced by any conventional means. See, for example, Houghten, Proc. Natl. Acad. Sci., USA 82:5131-5135 (1985), and U.S. Pat. No. 4,631,211 (1986).

Epitope-bearing peptides and polypeptides of the invention can be used to induce antibodies according to methods well known in the art. See, for instance, Bittle, et al, J Gen. Virol., 66:2347-2354 (1985). Immunogenic epitope-bearing peptides of the invention, i.e., those parts of a protein that elicit an antibody response when the whole protein is the immunogen, are identified according to methods known in the art. See, for instance, U.S. Pat. No. 5,194,392 (1990), which describes a general method of detecting or determining the sequence of monomers (amino acids or other compounds) which is a topological equivalent of the epitope (i.e., a “mimotope”) which is complementary to a particular paratope (antigen binding site) of an antibody of interest. More generally, U.S. Pat. No. 4,433,092 (1989) describes a method of detecting or determining a sequence of monomers which is a topographical equivalent of a ligand which is complementary to the ligand binding site of a particular receptor of interest. Similarly, U.S. Pat. No. 5,480,971 (1996) discloses linear C1-C7-alkyl peralkylated oligopeptides, and sets and libraries of such peptides, as well as methods for using such oligopeptide sets and libraries for determining the sequence of a peralkylated oligopeptide that preferentially binds to an acceptor molecule of interest. Thus, non-peptide analogs of the epitope-bearing peptides of the invention also can be made routinely by these methods.

Fusion Proteins

As one of skill in the art will appreciate, APO2L and IL-24 polypeptides of the present invention, and the epitope-bearing fragments thereof described above, can be combined with parts of the constant domain of immunoglobulins, resulting in chimeric polypeptides. These fusion proteins facilitate purification and show an increased half-life in vivo. This has been shown, e.g., for chimeric proteins consisting of the first two domains of the human CD4-polypeptide and various domains of the constant regions of the heavy or light chains of mammalian immunoglobulins, for example, EP A 394,827; Traunecker et al., Nature, 331:84-86 (1988). Fusion proteins that have a disulfide-linked dimeric structure due to the IgG part can also be more efficient in binding and neutralizing other molecules than the monomeric APO2L or IL-24 protein or protein fragment alone, for example, as described by Fountoulakis et al., J. Biochem., 270:3958-3964 (1995). Suitable chemical moieties for derivatization of a heterologous polypeptide include, for example, polymers, such as water soluble polymers, all or part of human serum albumin, fetuin A, fetuin B, leucine zipper nuclear factor erythroid derivative-2 (NFE2), neuroretinal leucine zipper, mannose motif (mbp1), tetranectin, and an Fc region.

Polymers, e.g., water soluble polymers, are useful in the present invention as the polypeptide to which each polymer is attached will not precipitate in an aqueous environment, such as a physiological environment. Polymers employed in the invention will be pharmaceutically acceptable for the preparation of a therapeutic product or composition. One skilled in the art will be able to select the desired polymer based on such considerations as whether the polymer/protein conjugate will be used therapeutically and, if so, the desired dosage, circulation time, and resistance to proteolysis.

Suitable, clinically acceptable, water soluble polymers include, but are not limited to, polyethylene glycol (PEG), polyethylene glycol propionaldehyde, copolymers of ethylene glycol/propylene glycol, monomethoxy-polyethylene glycol, carboxymethylcellulose, dextran, polyvinyl alcohol (PVA), polyvinyl pyrrolidone, poly-1,3-dioxolane, poly-1,3,6-trioxane, ethylene/maleic anhydride copolymer, poly (β-amino acids) (either homopolymers or random copolymers), poly(n-vinyl pyrrolidone) polyethylene glycol, polypropylene glycol homopolymers (PPG) and other polyakylene oxides, polypropylene oxide/ethylene oxide copolymers, polyoxyethylated polyols (POG) (e.g., glycerol) and other polyoxyethylated polyols, polyoxyethylated sorbitol, or polyoxyethylated glucose, colonic acids or other carbohydrate polymers, Ficoll, or dextran and mixtures thereof.

As used herein, polyethylene glycol (PEG) is meant to encompass any of the forms that have been used to derivatize other proteins, such as mono-(C1-C10) alkoxy- or aryloxy-polyethylene glycol. Polyethylene glycol propionaldehyde may have advantages in manufacturing due to its stability in water.

Specifically, a modified heterologous polypeptide of the invention may be prepared by attaching polyaminoacids or branch point amino acids to the polypeptide. For example, the polyaminoacid may be a carrier protein that serves to increase the circulation half life of the polypeptide (i.e., in addition to the advantages achieved via a fusion molecule). For the therapeutic purpose of the present invention, such polyaminoacids should ideally be those that have or do not create neutralizing antigenic response, or other adverse responses. Such polyaminoacids may be selected from serum album (such as human serum albumin), an additional antibody or portion thereof, for example the Fc region, fetuin A, fetuin B, leucine zipper nuclear factor erythroid derivative-2 (NFE2), neuroretinal leucine zipper, mannose motif (mbp1), tetranectin, or other polyaminoacids, e.g. lysines. As described herein, the location of attachment of the polyaminoacid may be at the N-terminus, or C-terminus, or other places in between, and also may be connected by a chemical “linker” moiety to the selected molecule.

Polymers used herein, for example water soluble polymers, may be of any molecular weight and may be branched or unbranched. The polymers each typically have an average molecular weight of between about 2 kDa to about 100 kDa (the term “about” indicating that in preparations of a polymer, some molecules will weigh more, some less, than the stated molecular weight). The average molecular weight of each polymer may be between about 5 kDa and about 50 kDa, or between about 12 kDa and about 25 kDa. Generally, the higher the molecular weight or the more branches, the higher the polymer:protein ratio. Other sizes may also be used, depending on the desired therapeutic profile; for example, the duration of sustained release; the effects, if any, on biological activity; the ease in handling; the degree or lack of antigenicity; and other known effects of a polymer on a modified molecule of the invention.

Polymers employed in the present invention are typically attached to a heterologous polypeptide with consideration of effects on functional or antigenic domains of the polypeptide. In general, chemical derivatization may be performed under any suitable condition used to react a protein with an activated polymer molecule. Activating groups which can be used to link the polymer to the active moieties include the following: sulfone, maleimide, sulfliydryl, thiol, triflate, tresylate, azidirine, oxirane, and 5-pyridyl.

Polymers of the invention are typically attached to a heterologous polypeptide at the alpha (α) or epsilon (ε) amino groups of amino acids or a reactive thiol group, but it is also contemplated that a polymer group could be attached to any reactive group of the protein that is sufficiently reactive to become attached to a polymer group under suitable reaction conditions. Thus, a polymer may be covalently bound to a heterologous polypeptide via a reactive group, such as a free amino or carboxyl group. The amino acid residues having a free amino group may include lysine residues and the N-terminal amino acid residue. Those having a free carboxyl group may include aspartic acid residues, glutamic acid residues, and the C-terminal amino acid residue. Those having a reactive thiol group include cysteine residues.

Methods for preparing fusion molecules conjugated with polymers, such as water soluble polymers, will each generally involve (a) reacting a heterologous polypeptide with a polymer under conditions whereby the polypeptide becomes attached to one or more polymers and (b) obtaining the reaction product. Reaction conditions for each conjugation may be selected from any of those known in the art or those subsequently developed, but should be selected to avoid or limit exposure to reaction conditions such as temperatures, solvents, and pH levels that would inactivate the protein to be modified. In general, the optimal reaction conditions for the reactions will be determined case-by-case based on known parameters and the desired result. For example, the larger the ratio of polymer:polypeptide conjugate, the greater the percentage of conjugated product. The optimum ratio (in terms of efficiency of reaction in that there is no excess unreacted polypeptide or polymer) may be determined by factors such as the desired degree of derivatization (e.g., mono-, di-tri- etc.), the molecular weight of the polymer selected, whether the polymer is branched or unbranched and the reaction conditions used. The ratio of polymer (e.g., PEG) to a polypeptide will generally range from 1:1 to 100:1. One or more purified conjugates may be prepared from each mixture by standard purification techniques, including among others, dialysis, salting-out, ultrafiltration, ion-exchange chromatography, gel filtration chromatography, and electrophoresis.

One may specifically desire an N-terminal chemically modified protein. One may select a polymer by molecular weight, branching, etc., the proportion of polymers to protein (polypeptide or peptide) molecules in the reaction mix, the type of reaction to be performed, and the method of obtaining the selected N-terminal chemically modified protein. The method of obtaining the N-terminal chemically modified protein preparation (i.e., separating this moiety from other monoderivatized moieties if necessary) may be by purification of the N-terminal chemically modified protein material from a population of chemically modified protein molecules.

Selective N-terminal chemical modification may be accomplished by reductive alkylation which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved. For example, one may selectively attach a polymer to the N-terminus of the protein by performing the reaction at a pH which allows one to take advantage of the pKa differences between the ε-amino group of the lysine residues and that of the α-amino group of the N-terminal residue of the protein. By such selective derivatization, attachment of a polymer to a protein is controlled: the conjugation with the polymer takes place predominantly at the N-terminus of the protein and no significant modification of other reactive groups, such as the lysine side chain amino groups, occurs. Using reductive alkylation, the polymer may be of the type described above and should have a single reactive aldehyde for coupling to the protein. Polyethylene glycol propionaldehyde, containing a single reactive aldehyde, may also be used.

In one embodiment, the present invention contemplates the chemically derivatized polypeptide to include mono- or poly-(e.g., 2-4) PEG moieties. Pegylation may be carried out by any of the pegylation reactions known in the art. Methods for preparing a pegylated protein product will generally include (a) reacting a polypeptide with polyethylene glycol (such as a reactive ester or aldehyde derivative of PEG) under conditions whereby the protein becomes attached to one or more PEG groups; and (b) obtaining the reaction product(s). In general, the optimal reaction conditions for the reactions will be determined case by case based on known parameters and the desired result.

There are a number of PEG attachment methods available to those skilled in the art. See, for example, EP 0 401 384; Malik et al., Exp. Heinatol., 20:1028-1035 (1992); Francis, Focus on Growth Factors, 3(2):4-10 (1992); EP 0 154 316; EP 0 401 384; WO 92/16221; WO 95/34326; and the other publications cited herein that relate to pegylation, the disclosures of which are hereby incorporated by reference.

The step of pegylation as described herein may be carried out via an acylation reaction or an alkylation reaction with a reactive polyethylene glycol molecule. Thus, protein products according to the present invention include pegylated proteins wherein the PEG group(s) is (are) attached via acyl or alkyl groups. Such products may be mono-pegylated or poly-pegylated (e.g., containing 2-6 or 2-5 PEG groups). The PEG groups are generally attached to the protein at the α- or ε-amino groups of amino acids, but it is also contemplated that the PEG groups could be attached to any amino group attached to the protein that is sufficiently reactive to become attached to a PEG group under suitable reaction conditions.

Pegylation by acylation generally involves reacting an active ester derivative of polyethylene glycol (PEG) with a polypeptide of the invention. For acylation reactions, the polymer(s) selected typically have a single reactive ester group. Any known or subsequently discovered reactive PEG molecule may be used to carry out the pegylation reaction. An example of a suitable activated PEG ester is PEG esterified to N-hydroxysuccinimide (NHS). As used herein, acylation is contemplated to include, without limitation, the following types of linkages between the therapeutic protein and a polymer such as PEG: amide, carbamate, urethane, and the like, see for example, Chamow, Bioconjugate Chem., 5:133-140 (1994). Reaction conditions may be selected from any of those known in the pegylation art or those subsequently developed, but should avoid conditions such as temperature, solvent, and pH that would inactivate the polypeptide to be modified.

Pegylation by acylation will generally result in a poly-pegylated protein. The connecting linkage may be an amide. The resulting product may be substantially only (e.g., >95%) mono, di- or tri-pegylated. However, some species with higher degrees of pegylation may be formed in amounts depending on the specific reaction conditions used. If desired, more purified pegylated species may be separated from the mixture (particularly unreacted species) by standard purification techniques, including among others, dialysis, salting-out, ultrafiltration, ion-exchange chromatography, gel filtration chromatography and electrophoresis.

Pegylation by alkylation generally involves reacting a terminal aldehyde derivative of PEG with a polypeptide in the presence of a reducing agent. For the reductive alkylation reaction, the polymer(s) selected should have a single reactive aldehyde group. An exemplary reactive PEG aldehyde is polyethylene glycol propionaldehyde, which is water stable, or mono C1-C10 alkoxy or aryloxy derivatives thereof, see for example, U.S. Pat. No. 5,252,714.

Additionally, heterologous polypeptides of the present invention and the epitope-bearing fragments thereof described herein can be combined with parts of the constant domain of immunoglobulins (IgG), resulting in chimeric polypeptides. These particular fusion molecules facilitate purification and show an increased half-life in vivo. This has been shown, e.g., for chimeric proteins consisting of the first two domains of the human CD4-polypeptide and various domains of the constant regions of the heavy or light chains of mammalian immunoglobulins, for example, EP A 394,827; Traunecker et al., Nature, 331:84-86 (1988). Fusion molecules that have a disulfide-linked dimeric structure due to the IgG part can also be more efficient in binding and neutralizing other molecules than, for example, a monomeric polypeptide or polypeptide fragment alone, see, for example, Fountoulakis et al., J. Biochem., 270:3958-3964 (1995).

In another described embodiment, a human serum albumin fusion molecule may also be prepared as described herein and as further described in U.S. Pat. No. 6,686,179.

Moreover, the polypeptides of the present invention can be fused to marker sequences, such as a peptide that facilitates purification of the fused polypeptide. In preferred embodiments, the marker amino acid sequence is a hexa-histidine peptide such as the tag provided in a pQE vector (QIAGEN, Inc., among others, many of which are commercially available. As described in Gentz et al., Proc. Natl. Acad. Sci. USA 86:821-824 (1989), for instance, hexa-histidine provides for convenient purification of the fusion protein. Another peptide tag useful for purification, the chemagglutinin HA tag, corresponds to an epitope derived from the influenza hemagglutinin protein. (Wilson et al., Cell 37:767 (1984)).

Thus, any of these above fusions can be engineered using the polynucleotides or the polypeptides of the present invention.

Secretory Leader Sequences

As demonstrated herein, and in U.S. 60/647,013, in order for some secreted proteins to express and secrete in larger quantities, a secretory leader sequence from another, i.e., different, secreted protein is desirable. Employing heterologous secretory leader sequences is advantageous in that a resulting mature amino acid sequence, i.e., protein, of the secreted polypeptide is not altered as the secretory leader sequence is removed in the ER during the secretion process. Moreover, the addition of a heterologous secretory leader is often required to express and secrete, for example, extracellular domains of Type II single transmembrane proteins (STM), as the secretory leader, which is also a transmembrane spanning domain, must typically be removed so that they may be soluble.

Thus, to identify potential robust secretory leader sequence(s) that could universally be used to secrete proteins and to express the intracellular domain of Type II STMs, Applicants have cloned and expressed, as described herein, a number of different secreted proteins and measured their expression and secretion levels in the supernatant of 293 mammalian cells. Several high expressers and high secretor proteins were observed.

In one embodiment, secretory leader sequences belonging to the secreted protein collagen type IX alpha I chain, long form was selected to further examine its ability to promote expression and secretion when used as a heterologous secretory leader sequence. As described herein, the amino acid sequence of the secreted protein collagen type IX alpha I chain, long form is predicted to be MKTCWKIPVFFFVCSFLEPWASA (SEQ ID NO.:26). As further described herein, vectors were constructed containing this particular secretory leader, several proteins were cloned removing the secretory leader from the full length encoding sequence, and by cloning them into vectors containing SEQ ID NO.: 26, resulting in secreted proteins with a heterologous' secretory leader sequence. As further shown and described herein, high expression and secretion of several other selected proteins were also observed.

As described herein, Applicants have identified secretory leader sequences from secreted proteins useful for producing proteins in higher yields than when such proteins are produced in their natural environment. Identified secretory leader sequences, described herein include, for example, interleukin-9 precursor, T cell growth factor P40, P40 cytokine, triacylglycerol lipase, pancreatic precursor, somatoliberin precursor, vasopressin-neurophysin 2-copeptin precursor, beta-enoendorphin-dynorphin precursor, complement C2 precursor, small inducible cytokine A14 precursor, elastase 2A precursor, plasma serine protease inhibitor precursor, granulocyte-macrophage colony-stimulating factor precursor, interleukin-2 precursor, interleukin-3 precursor, alpha-fetoprotein precursor, alpha-2-HS-glycoprotein precursor, serum albumin precursor, inter-alpha-trypsin inhibitor light chain, serum amyloid P-component precursor, apolipoprotein A-II precursor, apolipoprotein D precursor, colipase precursor, carboxypeptidase A1 precursor, alpha-s1 casein precursor, beta casein precursor, cystatin SA precursor, follitropin beta chain precursor, glucagon precursor, complement factor H precursor, histidine-rich glycoprotein precursor, interleukin-5 precursor, alpha-lactalbumin precursor, Von Ebner's gland protein precursor, matrix Gla-protein precursor, alpha-1-acid glycoprotein 2 precursor, phospholipase A2 precursor, dendritic cell chemokine 1, statherin precursor, transthyretin precursor, apolipoprotein A-1 precursor, apolipoprotein C-III precursor, apolipoprotein E precursor, complement component C8 gamma chain precursor, serotransferrin precursor, beta-2-microglobulin precursor, neutrophils defensins 1 precursor, triacylglycerol lipase gastric precursor, haptoglobin precursor, neutrophils defensins 3 precursor, neuroblastoma suppressor of tumorigenicity 1 precursor, small inducible cytokine A13 precursor, CD5 antigen-like precursor, phospholipids transfer protein precursor, dickkopf related protein-4 precursor, elastase 2B precursor, alpha-1-acid glycoprotein 1 precursor, beta-2-glycoprotein 1 precursor, neutrophil gelatinase-associated lipocalin precursor, C-reactive protein precursor, interferon gamma precursor, kappa casein precursor, plasma retinol-binding protein precursor, interleukin-13 precursor, and any of the secreted proteins set forth in the Tables or Sequence Listing.

The above-identified secretory leader sequences, vectors, and methods described herein, are useful in the expression of a wide variety of polypeptides, including, for example, secreted polypeptides, extracellular proteins, transmembrane proteins, and receptors, such as a soluble receptor. Examples of such polypeptides include cytokines and growth factors, such as interleukins 1 through 18, the interferons, the lympholkines, hormones, RANTES, lymphotoxin-β, Fas ligand, flt-3 ligand, ligand for receptor activator of NF-kappa B (RANKL), soluble receptors, TNF-related apoptosis-inducing ligand (TRAIL), CD40 ligand, O×40 ligand, 4-1BB ligand (and other members of the TNF family), thymic stroma-derived lymphopoietin, stimulatory factors, such as, for example, granulocyte colony stimulating factor and granulocyte-macrophage colony stimulating factor, inhibitory factors, mast cell growth factor, stem cell growth factor, epidermal growth factor, growth hormone, tumor necrosis factor, leukemia inhibitory factor, oncostatin-M, splice variants, and hematopoietic factors such as erythropoietin and thrombopoietin.

Descriptions of some proteins that can be expressed according to the invention may be found in, for example, Human Cytokines: Handbook for Basic and Clinical Research, Vol. II (Aggarwal and Gutterman, eds. Blackwell Sciences, Cambridge Mass., 1998); Growth Factors: A Practical Approach (McKay and Leigh, eds., Oxford University Press Inc., New York, 1993) and The Cytokile Handbook (A W Thompson, ed.; Academic Press, San Diego Calif.; 1991).

Receptors for any of the aforementioned proteins may also be expressed using secretory leader sequences, vectors and methods described herein, including, for example, both forms of tumor necrosis factor receptor (referred to as p55 and p75), interleukin-1 receptors (type 1 and 2), interleukin-4 receptor, interleukin-15 receptor, interleukin-17 receptor, interleukin-18 receptor, granulocyte-macrophage colony stimulating factor receptor, granulocyte colony stimulating factor receptor, receptors for oncostatin-M and leukemia inhibitory factor, receptor activator of NF-kappa B (RANK), receptors for TRAIL, and receptors that comprise death domains, such as Fas or apoptosis-inducing receptor (AIR).

Other proteins that can be expressed using the secretory leader sequences, vectors and methods described herein include, for example, cluster of differentiation antigens (referred to as CD proteins), for example, those disclosed in Leukocyte Typing VI (Proceedings of the VIth International Workshop and Conference; Kishimoto, Kikutani et al., eds.; Kobe, Japan, 1996), or CD molecules disclosed in subsequent workshops. Examples of such molecules include CD27, CD30, CD39, CD40; and ligands thereto (CD27 ligand, CD30 ligand and CD40 ligand). Several of these are members of the TNF receptor family, which also includes 41BB and O×40; the ligands are often members of the TNF family (as are 4-11BB ligand and O×40 ligand); accordingly, members of the TNF and TNFR families can also be expressed using the present invention.

Proteins that are enzymatically active may also be expressed employing the herein described secretory leader sequences, vectors and methods and include, for example, metalloproteinase-disintegrin family members, various kinases (including streptokinase and tissue plasminogen activator as well as death associated kinase containing ankyrin repeats, and IKR 1 and 2), TNF-alpha converting enzyme, and numerous other enzymes. Ligands for enzymatically active proteins can also be expressed by applying the instant invention.

The secretory leader sequences, vectors, and methods described herein, are also useful for the expression of other types of recombinant proteins, including, for example, immunoglobulin molecules or portions thereof, and chimeric antibodies (i.e., an antibody having a human constant region couples to a murine antigen binding region) or fragments thereof. Numerous techniques are known by which DNA encoding immunoglobulin molecules can be manipulated to yield DNAs capable of encoding recombinant proteins such as single chain antibodies, antibodies with enhanced affinity, or other antibody-based polypeptides (see, for example, Larrick et al., Biotechnology 7:934-938, 1989; Reichmann et al., Nature 332:323-327, 1988; Roberts et al., Nature 328:731-734, 1987; Verhoeyen et al., Science 239:1534-1536, 1988; Chaudhary et al., Nature 339:394-397,1989).

Co-Translational and Post-Translational Modifications

The invention encompasses polypeptides which are differentially modified during or after translation, e.g., by glycosylation, acetylation, phosphorylation, amidation, derivatization by known protecting/blocking groups, proteolytic cleavage, linkage to an antibody molecule or other cellular ligand. Any of numerous chemical modifications may be carried out by known techniques, including but not limited to specific chemical cleavage by cyanogen bromide, trypsin, chymotrypsin, papain, V8 protease; NAB H₄; acetylation; formylation; oxidation; reduction; and/or metabolic synthesis in the presence of tunicamycin.

Additional post-translational modifications encompassed by the invention include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of procaryotic host cell expression. The polypeptides may also be modified with a detectable label, such as an enzymatic, fluorescent, isotopic, or affinity label to allow for detection and isolation of the protein.

Also provided by the invention are chemically modified derivatives of the polypeptides of the invention which may provide additional advantages such as increased solubility, stability, and circulating time of the polypeptide, or decreased immunogenicity (see U.S. Pat. No. 4,179,337). The chemical moieties for derivitization may be selected from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three, or more attached chemical moieties.

A polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

The polyethylene glycol molecules (or other chemical moieties) should be attached to the protein with consideration of effects on functional or antigenic domains of the protein. There are a number of attachment methods available to those skilled in the art, e.g., EP 0 401 384, herein incorporated by reference (coupling PEG to G-CSF), see also Malik et al., Exp. Hematol. 20:1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Suitable for therapeutic purposes is attachment at an amino group, such as attachment at the N-terminus or lysine group.

One may specifically desire proteins chemically modified at the N-terminus. Using polyethylene glycol as an illustration of the present composition, one may select from a variety of polyethylene glycol molecules (by molecular weight, branching, etc.), the proportion of polyethylene glycol molecules to protein (polypeptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus modification may be accomplished by reductive alkylation which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved.

### Compositions

In some embodiments, APO2L and IL-24 compositions are provided in formulation with pharmaceutically acceptable excipients, a wide variety of which are known in the art (Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3^{rd} ed., Pharmaceutical Press( 2000)). Pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In pharmaceutical dosage forms, the compositions of the invention can be administered in the form of their pharmaceutically acceptable salts, or they can also be used alone or in appropriate association, as well as in combination, with other pharmaceutically active compounds. The subject compositions are formulated in accordance to the mode of potential administration. Administration of the agents can be achieved in various ways, including oral, buccal, nasal, rectal, parenteral, intraperitoneal, intradermal, transdermal, subcutaneous, intravenous, intra-arterial, intracardiac, intraventricular, intracranial, intratracheal, and intrathecal administration, etc., or otherwise by implantation or inhalation. Thus, the subject compositions can be formulated into preparations in solid, semi-solid, liquid or gaseous forms, such as tablets, capsules, powders, granules, ointments, solutions, suppositories, injections, inhalants and aerosols. The following methods and excipients are merely exemplary and are in no way limiting.

Compositions for oral administration can form solutions, suspensions, tablets, pills, granules, capsules, sustained release formulations, oral rinses, or powders. For oral preparations, the agents, polynucleotides, and polypeptides can be used alone or in combination with appropriate additives, for example, with conventional additives, such as lactose, mannitol, corn starch, or potato starch; with binders, such as crystalline cellulose, cellulose derivatives, acacia, corn starch, or gelatins; with disintegrators, such as corn starch, potato starch, or sodium carboxymethylcellulose; with lubricants, such as talc or magnesium stearate; and if desired, with diluents, buffering agents, moistening agents, preservatives, and flavoring agents.

Suitable excipient vehicles are, for example, water, saline, dextrose, glycerol, ethanol, or the like, and combinations thereof. In addition, if desired, the vehicle can contain minor amounts of auxiliary substances such as wetting or emulsifying agents or pH buffering agents. Actual methods of preparing such dosage forms are known, or will be apparent, to those skilled in the art (Gennaro, 2003). The composition or formulation to be administered will, in any event, contain a quantity of the agent adequate to achieve the desired state in the subject being treated.

The agents, polynucleotides, and polypeptides can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. Other formulations for oral or parenteral delivery can also be used, as conventional in the art.

The antibodies, agents, polynucleotides, and polypeptides can be utilized in aerosol formulation to be administered via inhalation. The compounds of the present invention can be formulated into pressurized acceptable propellants such as dichlorodifluoromethane, propane, nitrogen, and the like. Further, the agent, polynucleotides, or polypeptide composition may be converted to powder form for administration intranasally or by inhalation, as conventional in the art.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

A polynucleotide, polypeptide, or other modulator, can also be introduced into tissues or host cells by other routes, such as viral infection, microinjection, or vesicle fusion. For example, expression vectors can be used to introduce nucleic acid compositions into a cell as described above. Further, jet injection can be used for intramuscular administration (Furth et al., Anal. Biochem. 205:365-368 (1992)). The DNA can be coated onto gold microparticles, and delivered intradermally by a particle bombardment device, or “gene gun” as described in the literature (Tang et al., Nature 356:152-154 (1992)), where gold microprojectiles are coated with the DNA, then bombarded into skin cells.

Unit dosage forms for oral or rectal administration such as syrups, elixirs, and suspensions can be provided wherein each dosage unit, for example, teaspoonful, tablespoonful, tablet, or suppository, contains a predetermined amount of the composition containing one or more agents. Similarly, unit dosage forms for injection or intravenous administration can comprise the agent(s) in a composition as a solution in sterile water, normal saline or another pharmaceutically acceptable carrier.

### Chromosome Assays

In certain embodiments relating to chromosomal mapping, a cDNA herein disclosed is used to clone the genomic nucleic acid of the APO2L or IL-24. This can be accomplished using a variety of well known techniques and libraries, which generally are commercially available. The genomic DNA then is used for in situ chromosome mapping using techniques well known for this purpose. Therefore, the nucleic acid molecules of the present invention are also valuable for chromosome identification. The sequence is specifically targeted to and can hybridize with a particular location on an individual human chromosome. Moreover, there is a current need for identifying particular sites on the chromosome. Few chromosome marking reagents based on actual sequence data (repeat polymorphisms) are presently available for marking chromosomal location. The mapping of DNAs to chromosomes according to the present invention is an important first step in correlating those sequences with genes associated with disease.

Briefly, sequences can be mapped to chromosomes by preparing PCR primers from the cDNA. Computer analysis of the 3′ untranslated region is used to rapidly select primers that do not span more than one exon in the genomic DNA, thus complicating the amplification process. These primers are then used for PCR screening of somatic cell hybrids containing individual human chromosomes. Only those hybrids containing the human gene corresponding to the primer will yield an amplified fragment.

PCR mapping of somatic cell hybrids is a rapid procedure for assigning a particular DNA to a particular chromosome. Using the present invention with the same oligonucleotide primers, sublocalization can be achieved with panels of fragments from specific chromosomes or pools of large genomic clones in an analogous manner. Other mapping strategies that can similarly be used to map to its chromosome include in situ hybridization, prescreening with labeled flow-sorted chromosomes and preselection by hybridization to construct chromosome specific-cDNA libraries.

Fluorescence in situ hybridization (FISH) of a cDNA clone to a metaphase Chromosomal spread can be used to provide a precise chromosomal location in one step. This technique can be used with a cDNA as short as approximately 50-60 bases. For a review of this technique, see Verma et al., Human Chromosomes: A Manual of Basic Techniques, Pergamon Press, New York (1988).

Once a sequence has been mapped to a precise chromosomal location, the physical position of the sequence on the chromosome can be correlated with genetic map data. (Such data are found, for example, in V. McKusick, Mendelian Inheritance in Man, available on line through Johns Hopkins University Welch Medical Library). The relationship between genes and diseases that have been mapped to the same chromosomal region are then identified through linkage analysis (coinheritance of physically adjacent genes).

Next, differences can be determined in the cDNA or genomic sequences of affected and unaffected individuals. If a mutation is observed in some or all of the affected individuals but not in any normal individuals, then the mutation is likely to be the causative agent of the disease. With current resolution of physical mapping and genetic mapping techniques, a cDNA precisely localized to a chromosomal region associated with the disease could be one of between 50 and 500 potential causative genes (assuming 1 megabase mapping resolution and one gene per 20 kb).

Using methods described above, the APO2L or IL-24 gene of the invention has been mapped by fluorescent in situ hybridization to human chromosome 8p21. The corresponding map position in the mouse includes several disease loci, including the ds (disorganization—developmental disruption) locus and the wc (waved coat-homozygous lethality) locus.

### Identification of Agonists and Antagonists

This invention provides modulators, i.e., polypeptides, polynucleotides, or other agents that increase or decrease the activity of their target. They may act as an agonist or antagonist, and interfere with the binding or activity of polypeptides or polynucleotides. Such modulators or agents include, for example, polypeptide variants, whether agonist or antagonist; antibodies, whether agonist or antagonist; soluble receptors, usually antagonists; small molecule drugs, whether agonist or antagonist; RNAi, usually an antagonist; antisense molecules, usually an antagonist; and ribozymes, usually an antagonist. In some embodiments, an agent is a subject polypeptide, where the subject polypeptide itself is administered to an individual. In some embodiments, an agent is an antibody specific for a subject “target” polypeptide. In some embodiments, an agent is a chemical compound such as a small molecule that may be useful as an orally available drug. Such modulation includes the recruitment of other molecules that directly effect the modulation. For example, an antibody that modulates the activity of a subject polypeptide that is a receptor on a cell surface may bind to the receptor and fix complement, activating the complement cascade and resulting in lysis of the cell. An agent which modulates a biological activity of a subject polypeptide or polynucleotide increases or decreases the activity or binding at least about 10%, at least about 15%, at least about 20%, at least about 25%, at least about 50%, at least about 80%, or at least about 2-fold, at least about 5-fold, or at least about 10-fold or more when compared to a suitable control.

This invention also provides a method of screening compounds to identify those which modulate the action of the polypeptide of the present invention. An example of such an assay comprises combining a mammalian fibroblast cell and the polypeptide(s) of the present invention, the compound to be screened and ³[H] thymidine under cell culture conditions where the fibroblast cell would normally proliferate. A control assay may be performed in the absence of the compound to be screened and compared to the amount of fibroblast proliferation in the presence of the compound to determine if the compound stimulates proliferation by determining the uptake of ³[H] thymidine in each case. The amount of fibroblast cell proliferation is measured by liquid scintillation chromatography, which measures the incorporation of ³[H] thymidine. Both agonistic and antagonistic compounds may be identified by this procedure.

In another method, a mammalian cell or membrane preparation expressing a receptor for a polypeptide of the present invention, as described above, is incubated with a labeled polypeptide of the present invention in the presence of the compound. The ability of the compound to enhance or block this interaction could then be measured. Alternatively, the response of a known second messenger system following interaction of a compound to be screened and the APO2L or IL-24 receptor is measured and the ability of the compound to bind to the receptor and elicit a second messenger response is measured to determine if the compound is a potential agonist or antagonist. Such second messenger systems include, but are not limited to, cAMP, guanylate cyclase, ion channels, and phosphoinositide hydrolysis.

Examples of antagonistic compounds include antibodies, or in some cases, oligonucleotides, which bind to a receptor of a polypeptide of the present invention but elicit no second messenger response, or which bind to the APO2L or IL-24 polypeptide itself. Alternatively, a potential antagonist may be a mutant form of the polypeptide which binds to the receptors but elicits no second messenger response, thus effectively blocking the action of the polypeptide.

Another compound antagonistic to APO2L or IL-24 genes and gene products is an antisense construct prepared using antisense technology. Antisense technology can be used to control gene expression through triple-helix formation or antisense DNA or RNA; both methods are based on the binding of a polynucleotide to DNA or RNA. For example, a 5′ coding portion of the polynucleotide sequence, which encodes mature polypeptides of the present invention, can be used to design an antisense RNA oligonucleotide of from about 10 to about 40 base pairs in length. A DNA oligonucleotide is designed to be complementary to a region of the gene involved in transcription, for example, a triple helix—see Lee et al., Nucl. Acids Res., 6:3073 (1979); Cooney et al, Science, 241:456 (1988); and Dervan et al., Science, 251: 1360 (1991), thereby preventing transcription and the production of the polypeptides of the present invention. The antisense RNA oligonucleotide hybridizes to the mRNA in vivo and blocks translation of the mRNA molecule into the polypeptide, as described by Okano, J. Neurochem., 56:560 (1991); Oligodeoxynucleotides as Antisense Inhibitors of Gene Expression, CRC Press, Boca Raton, Fla. (1988). The oligonucleotides described above can also be delivered to cells such that the antisense RNA or DNA is expressed in vivo to inhibit polypeptide production.

Potential antagonist compounds also include small molecules which bind to and occupy the binding site of the receptors, thereby making the receptor inaccessible to its polypeptide such that normal biological activity is prevented. Examples of small molecules include, but are not limited to, small peptides or peptide-like molecules. Antagonist compounds may be employed to inhibit the cell growth and proliferation effects of the polypeptides of the present invention on neoplastic cells and tissues, i.e. stimulation of angiogenesis of tumors, and, therefore, retard or prevent abnormal cellular growth and proliferation, for example, in tumor formation or growth.

The antagonists may also be employed to prevent hyper-vascular diseases, and prevent the proliferation of epithelial lens cells after extracapsular cataract surgery. Prevention of the mitogenic activity of the polypeptides of the present invention may also be desirous in cases such as restenosis after balloon angioplasty. The antagonists may also be employed to prevent the growth of scar tissue during wound healing.

The present invention also provides methods for identifying agents, such as antibodies, which enhance or block the actions of APO2L or IL-24 molecules on cells. For example these agents may enhance or block interaction of APO2L or IL-24-binding molecules, such as receptors. Agents of interest include both agonists and antagonists. The invention provides agonists which increase the natural biological functions of APO2L or IL-24 or which function in a manner similar to APO2L or IL-24. The invention also provides antagonists, which decrease or eliminate the functions of APO2L or IL-24.

One method of identifying APO2L and IL-24 agonists and antagonists involves biochemical assays following subcellular fractionation. For example, a cellular compartment, such as a membrane or cytosolic preparation may be prepared from a cell that expresses a molecule that binds APO2L or IL-24 molecules, such as a molecule of a signaling or regulatory pathway modulated by APO2L or IL-24 molecules. Subcellular fractionation methods are known in the art of cell biology, and can be tailored to produce crude fractions with discrete and defined components, e.g., organelles or organellar membranes. The preparation is incubated with labeled APO2L and IL-24 molecules in the absence or the presence of a candidate molecule which may be an APO2L and IL-24 agonist or antagonist. The ability of the candidate molecule to interact with the binding molecule or an APO2L or IL-24 molecules is reflected in decreased binding of the labeled ligand. Molecules which bind gratuitously, that is, without inducing the effects of APO2L or IL-24 molecules, are most likely antagonists. Molecules that bind well and elicit effects that are the same as or closely related to APO2L and/or IL-24 molecules may potentially prove to be agonists.

The effects of potential agonists and antagonists may by measured, for instance, by determining an activity of one or more components of a second messenger system following interaction of the candidate molecule with a cell or appropriate cell preparation, and comparing the effect with that of APO2L or IL-24 molecules, or with that of molecules that elicit the same effects as APO2L and IL-24. Second messenger systems which may be useful in this regard include, but are not limited to, cAMP, cGMP, ion channel, and phosphoinositide hydrolysis second messenger systems.

Another example of an assay for the identification of APO2L and/or IL-24 antagonists is a competitive assay that combines a mixture of APO2L or IL-24 molecules and a potential antagonist, with membrane-bound APO2L or IL-24 receptor molecules. Under appropriate conditions for a competitive inhibition assay, this assay can also be performed with recombinant APO2L or IL-24 receptor molecules. APO2L or IL-24 molecules can be labeled, such as by radioactivity, such that the number of APO2L or IL-24 molecules bound to a receptor molecule can be determined accurately to assess the effectiveness of the potential antagonist.

Potential antagonists include small organic molecules, polypeptides, and antibodies that bind to a polypeptide of the invention, and thereby inhibit or extinguish its activity. Potential antagonists also may be small organic molecules, polypeptides such as closely related proteins or antibodies that bind the same sites on a binding molecule, such as a receptor molecule, without inducing APO2L or IL-24-induced activities, thereby preventing the action of APO2L or IL-24 molecules by excluding APO2L or IL-24 molecules from binding. Antagonists of the invention include fragments of the APO2L and IL-24 molecules having the nucleic acid and amino acid sequences shown in the Sequence Listing.

Other potential antagonists include antisense molecules. Antisense technology can be used to control gene expression through, e.g., antisense DNA or RNA, or through triple-helix formation. Antisense techniques are discussed, for example, in Okano, J. Neurochem., 56:560 (1991); Oligodeoxynucleotides as Antisense Inhibitors of Gene Expression, CRC Press, Boca Raton, Fla. (1988). Triple helix formation is discussed in, for instance, Lee et al., Nucleic Acids Research, 6:3073 (1979); Cooney et al., Science, 241:456 (1988); and Dervan et al., Science, 251:1360 (1991). The methods are based on the binding of a polynucleotide to a complementary DNA or RNA. For example, the 5′ coding portion of a polynucleotide that encodes the mature polypeptide of the present invention may be used to design an antisense RNA oligonucleotide of from about 10 to about 40 base pairs in length. A DNA oligonucleotide is designed to be complementary to a region of the gene involved in transcription, thereby preventing transcription and the subsequent production of APO2L or IL-24 molecules. The antisense RNA oligonucleotide hybridizes to the mRNA in vivo and blocks translation of the mRNA molecule into an APO2L or IL-24 polypeptide. The oligonucleotides described above can also be delivered to cells such that the antisense RNA or DNA may be expressed in vivo to inhibit production of APO2L or IL-24 molecules.

### Therapeutic Uses of APO2L and IL-24, and Their Agonists and Antagonists

APO2L or IL-24 polynucleotides, polypeptides, agonists, and/or antagonists of the invention may be used in developing treatments for any disorder mediated (directly or indirectly) by defective APO2L or IL-24 molecules, or insufficient amounts of either of these. APO2L or IL-24 polypeptides, agonists, and/or antagonists may be administered to a patient (e.g., a mammal, such as human) afflicted with such a disorder. Alternatively, a gene therapy approach may be applied to treat such disorders. Disclosure herein of APO2L and IL-24 nucleotide sequences permits the detection of defective APO2L and IL-24 genes, and the replacement thereof with normal APO2L and IL-24-encoding genes. Defective genes may be detected in in vitro diagnostic assays, and by comparison of the APO2L and IL-24 nucleotide sequences disclosed herein with that of an APO2L and IL-24 gene derived from a patient suspected of harboring a defect in this gene.

The APO2L and/or IL-24 molecules of the present invention may be employed to treat lymphoproliferative disease which results in lymphadenopathy. They may also mediate apoptosis by stimulating clonal deletion of T-cells and may, therefore, be employed to treat autoimmune disease to stimulate peripheral tolerance and cytotoxic T-cell mediated apoptosis. The APO2L and/or IL-24 molecules may further be employed as a research tool in elucidating the biology of autoimmune disorders, including systemic lupus erythematosus (SLE), Graves' disease, immunoproliferative disease lymphadenopathy (IPL), angioimmunoproliferative lymphadenopathy (AIL), immunoblastic lymphadenopathy (IBL), rheumatoid arthritis, diabetes, and multiple sclerosis. They also find use in treating allergies and graft versus host disease.

The APO2L and IL-24 polynucleotides, polypeptides, and/or agonists or antagonists of the invention may also be used to treat, prevent, diagnose, and/or prognose diseases which include, but are not limited to, autoimmune disorders, immunodeficiency disorders, and graft versus host disease. Particular types of autoimmune diseases that can be treated with the molecules of the invention include, but are not limited to, Th2-lymphocyte related disorders (e.g., atopic dermatitis, atopic asthma, rhinoconjunctivitis, allergic rhinitis, Omenn's syndrome, systemic sclerosis, and graft versus host disease); Th-1 lymphocyte-related disorders (e.g., rheumatoid arthritis, multiple sclerosis, psoriasis, Sjögren's syndrome, Hashimoto's thyroiditis, Grave's disease, primary biliary cirrhosis, Wegener's granulomatosis, and tuberculosis); activated B lymphocyte-related disorders (e.g., systemic lupus erythematosus, Goodpasture's syndrome, rheumatoid arthritis, and type I diabetes).

The APO2L and IL-24 polypeptides of the present invention may be employed to inhibit neoplasia, such as tumor cell growth. They may be responsible for tumor destruction through apoptosis and cytotoxicity to certain cells. Multidrug resistant osteosarcomas are sensitive to APO2L, and the response to APO2L correlates with the expression of Akt (Cenni et al., Int. J. Oncol. 25:1599-1608 (2004)).

Diseases associated with increased cell survival, or the inhibition of apoptosis, that may be treated, prevented, diagnosed and/or prognosed with the APO2L or IL-24 polynucleotides, polypeptides and/or agonists or antagonists of the invention include, but are not limited to, cancers (such as follicular lymphomas, carcinomas with p53 mutations, and hormone-dependent tumors, including, but not limited to colon cancer, cardiac tumors, pancreatic cancer, melanoma, retinoblastoma, glioblastoma, lung cancer, intestinal cancer, testicular cancer, stomach cancer, neuroblastoma, myxoma, myoma, lymphoma, endothelioma, osteoblastoma, osteoclastoma, osteosarcoma, chondrosarcoma, adenoma, breast cancer, prostate cancer, Kaposi's sarcoma, and ovarian cancer); autoimmune disorders (such as, multiple sclerosis, Sjögren's syndrome, Graves' disease, Hashimoto's thyroiditis, autoimmune diabetes, biliary cirrhosis, Behcet's disease, Crohn's disease, polymyositis, systemic lupus erythematosus, and immune-related glomerulonephritis, autoimmune gastritis, autoimmune thrombocytopenic purpura, and rheumatoid arthritis) and viral infections (such as herpes viruses, pox viruses and adenoviruses), inflammation, graft vs. host disease (acute and/or chronic), acute graft rejection, and chronic graft rejection. In preferred embodiments, APO2L or IL-24 polynucleotides and/or polypeptides, and their agonists, and/or antagonists are used to inhibit growth, progression, and/or metastasis of cancers, in particular those listed above or in the paragraph that follows.

Additional diseases or conditions associated with increased cell survival, that may be treated, prevented, diagnosed, and/or prognosed with the APO2L or IL-24 polynucleotides and/or polypeptides and their agonists and/or antagonists include, but are not limited to, progression and/or metastases of malignancies and related disorders such as leukemia (including acute leukemias, (e.g., acute lymphocytic leukemia and acute myelocytic leukemia (including myeloblastic, promyelocytic, myelomonocytic, monocytic, and erythroleukemia) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain diseases, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma. For example, APO2L induces selective apoptosis in most cell lines derived from the Ewing's sarcoma family of tumors (Merchant et al., Cancer Res. 64:8349-56 (2004)).

Diseases associated with increased apoptosis, that may be treated, prevented, diagnosed and/or prognosed with the APO2L or IL-24 polynucleotides, polypeptides and/or agonists or antagonists of the invention include, but are not limited to, AIDS, neurodegenerative disorders (such as Alzheimer's disease, Parkinson's disease, amyotrophic lateral sclerosis, retinitis pigmentosa, cerebellar degeneration, and brain tumor or prior associated disease); diabetes, autoimmune disorders (such as, multiple sclerosis, Sjögren's syndrome, Graves' disease, Hashimoto's thyroiditis, autoimmune diabetes, biliary cirrhosis, Behcet's disease, Crohn's disease, polymyositis, SLE, immune-related glomerulonephritis, autoimmune gastritis, thrombocytopenic purpura, and rheumatoid arthritis) myelodysplastic syndromes (such as aplastic anemia), graft vs. host disease (acute and/or chronic), ischemic injury (such as that caused by myocardial infarction, stroke, and reperfusion injury), liver injury or disease (e.g., hepatitis related liver injury, cirrhosis, ischemia/reperfusion injury, cholestosis (bile duct injury) and liver cancer); toxin-induced liver disease (such as that caused by alcohol), septic shock, ulcerative colitis, cachexia, and anorexia. In some embodiments, APO2L or IL-24 polynucleotides, polypeptides, agonists, and/or antagonists are used to treat the diseases and disorders listed above.

Molecules of the invention are useful for killing or inhibiting the multiplication of a cell that produces an infectious disease or for treating an infectious disease. The molecules of the invention can be used accordingly in a variety of settings for the treatment of an infectious disease in an animal. In the context of an infectious disease, the term “treating” includes any or all of preventing the growth, multiplication or replication of the pathogen that causes the infectious disease and ameliorating one or more symptoms of an infectious disease.

Many of the pathologies associated with HIV are mediated by apoptosis, including HIV-induced nephropathy and HIV encephalitis. Thus, in some embodiments, APO2L or IL-24 polynucleotides, polypeptides, agonists or antagonists of the invention are used to treat AIDS and pathologies associated with AIDS.

Another embodiment of the present invention is directed to the use of APO2L or IL-24 polynucleotides, polypeptides, or antagonists to reduce APO2L or IL-24-mediated death of T cells in HIV-infected patients. The role of T cell apoptosis in the development of AIDS has been the subject of a number of studies (see, for example, Meyaard et al., Science, 257:217-219 (1992); Groux et al., J. Exp. Med., 175:331 (1992); and Oyaizu et al., in Cell Activation and Apoptosis in HIV Infection, Andrieu and Lu, Eds., Plenum Press, New York, pp. 101-114 (1995)). Fas-mediated apoptosis has been implicated in the loss of T cells in HIV positive individuals (Katsikis et al., J. Exp. Med. 181:2029-2036 (1995). It is also likely that T cell apoptosis occurs through multiple mechanisms.

APO2L and/or IL-24 polypeptides of the invention may also be employed to regulate hematopoiesis and, in particular, erythropoiesis. Hematopoiesis is a multi-step cell proliferation and differentiation process which begins with a pool of multipotent stem cells. These cells can proliferate and differentiate into hematopoietic progenitors in reply to different stimuli. The APO2L and/or IL-24 polypeptides of the invention, as well as agonists and antagonists thereof, may be used to either stimulate or inhibit development of hematopoietic cells and, in particular, erythropoietic precursor cells.

Additionally, molecules of the invention may be employed as agents to boost immunoresponsiveness among individuals having a temporary immune deficiency. Conditions resulting in a temporary immune deficiency that may be ameliorated or treated by administering the APO2L or IL-24 polypeptides or polyniucleotides of the invention, or agonists thereof, include, but are not limited to, recovery from viral infections (e.g., influenza), conditions associated with malnutrition, recovery from infectious mononucleosis, or conditions associated with stress, recovery from measles, recovery from blood transfusion, and recovery from surgery.

In the context of an autoimmune disease, the term “treating” includes any or all of preventing replication of cells associated with an autoimmune disease state including, but not limited to, cells capable of producing an autoimmune antibody, lessening the autoimmune-antibody burden, and ameliorating one or more symptoms of an autoimmune disease.

APO2L or IL-24 polynucleotides or polypeptides of the invention, or agonists or antagonists thereof, may be used to diagnose, prognose, treat, or prevent one or more of the following diseases or disorders, or conditions associated therewith: primary immunodeficiencies, immune-mediated thrombocytopenia, Kawasaki syndrome, bone marrow transplant (e.g., recent bone marrow transplant in adults or children), chronic B-cell lymphocytic leukemia, HIV infection (e.g., adult or pediatric HIV infection), chronic inflammatory deniyelinating polyneuropathy, and post-transfusion purpura.

Additionally, APO2L or IL-24 polynucleotides or polypeptides of the invention, or agonists or antagonists thereof, may be used to diagnose, prognose, treat or prevent one or more of the following diseases, disorders, or conditions associated therewith, Guillain-Barre syndrome, anemia (e.g., anemia associated with parvovirus B19, patients with stable multiple myeloma who are at high risk for infection (e.g., recurrent infection), autoimmune hemolytic anemia (e.g., warm-type autoimmune hemolytic anemia), thrombocytopenia (e.g., neonatal thrombocytopenia), and immune-mediated neutropenia), transplantation (e.g., cytomegalovirus (CMV)-negative recipients of CMV-positive organs), hypogammaglobulinemia (e.g., hypogammaglobulinemic neonates with risk factor for infection or morbidity), epilepsy (e.g., intractable epilepsy), systemic vasculitic syndromes, myasthenia gravis (e.g., decompensation in myasthenia gravis), dermatomyositis, and polymyositis.

Autoimmune disorders and conditions associated with these disorders that may be treated, prevented, and/or diagnosed with the APO2L or IL-24 polynucleotides, polypeptides, and/or antagonist of the invention (e.g., anti-APO2L or IL-24 antibodies), include, but are not limited to, autoimmune hemolytic anemia, autoimmune neonatal thrombocytopenia, idiopathic thrombocytopenia purpura, autoimmunocytopenia, hemolytic anemia, antiphospholipid syndrome, dermatitis, allergic encephalomyelitis, myocarditis, relapsing polychondritis, rheumatic heart disease, glomerulonephritis (e.g., IgA nephropathy), multiple sclerosis, neuritis, uveitis ophthalmia, polyendocrinopathies, purpura (e.g., Henloch-Scoenlein purpura), Reiter's disease, stiff-man syndrome, autoimmune pulmonary inflammation, Guillain-Barre syndrome, insulin dependent diabetes mellitus, and autoimmune inflammatory eye disease.

Additional autoimmune disorders highly likely to be treated, prevented, and/or diagnosed with the compositions of the invention include, but are not limited to, autoimmune thyroiditis, hypothyroidism (i.e., Hashimoto's thyroiditis) (often characterized, e.g., by cell-mediated and humoral thyroid cytotoxicity), systemic lupus erythematosus (often characterized, e.g., by circulating and locally generated immune complexes), Goodpasture's syndrome (often characterized, e.g., by anti-basement membrane antibodies), pemphigus (often characterized, e.g., by epidermal acantholytic antibodies), receptor autoimmunities such as, for example, (a) Graves' disease (often characterized, e.g., by TSH receptor antibodies), (b) myasthenia gravis (often characterized, e.g., by acetylcholine receptor antibodies), and (c) insulin resistance (often characterized, e.g., by insulin receptor antibodies), autoimmune hemolytic anemia (often characterized, e.g., by phagocytosis of antibody-sensitized red blood cells), autoimmune thrombocytopenic purpura (often characterized, e.g., by phagocytosis of antibody-sensitized platelets.

Additional autoimmune disorders which may be treated, prevented, and/or diagnosed with the compositions of the invention include, but are not limited to, rheumatoid arthritis (often characterized, e.g., by immune complexes in joints), scleroderma with anti-collagen antibodies (often characterized, e.g., by nucleolar and other nuclear antibodies), mixed connective tissue disease (often characterized, e.g., by antibodies to extractable nuclear antigens (e.g., ribonucleoprotein)), polymyositis/dermatomyositis (often characterized, e.g., by nonhistone anti-nuclear antibodies), pernicious anemia (often characterized, e.g., by antibodies to parietal cells, microsomes, and intrinsic factor), idiopathic Addison's disease (often characterized, e.g., by humoral and cell-mediated adrenal cytotoxicity, infertility (often characterized, e.g., by antispermatozoal antibodies), glomerulonephritis (often characterized, e.g., by glomerular basement membrane antibodies or immune complexes) such as primary glomerulonephritis and IgA nephropathy, bullous pemphigoid (often characterized, e.g., by IgG and complement in the basement membrane), Sjögren's syndrome (often characterized, e.g., by multiple tissue antibodies, and/or a specific nonhistone anti-nuclear antibodies (SS-B)), diabetes mellitus (often characterized, e.g., by cell-mediated and humoral islet cell antibodies), and adrenergic drug resistance (including adrenergic drug resistance with asthma or cystic fibrosis) (often characterized, e.g., by beta-adrenergic receptor antibodies).

Further autoimmune disorders which may be treated, prevented, and/or diagnosed with the compositions of the invention include, but are not limited to, chronic active hepatitis (often characterized, e.g. by smooth muscle antibodies), primary biliary cirrhosis (often characterized, e.g., by mitochondrial antibodies), other endocrine gland failure (often characterized, e.g., by specific tissue antibodies in some cases), vitiligo (often characterized, e.g., by melanocyte antibodies), vasculitis (often characterized, e.g., by Ig and complement in vessel walls and/or low serum complement), post-myocardial infarction (often characterized, e.g., by myocardial antibodies), cardiotomy syndrome (often characterized, e.g., by myocardial antibodies), urticaria (often characterized, e.g., by IgG and IgM antibodies to IgE), atopic dermatitis (often characterized, e.g., by IgG and IgM antibodies to IgE), asthma (often characterized, e.g., by IgG and IgM antibodies to IgE), inflammatory myopathies, and many other inflammatory, granulamatous, degenerative, and atrophic disorders.

In an additional embodiment, APO2L or IL-24 polynucleotides or polypeptides, or antagonists thereof (e.g., anti-APO2L or IL-24 antibodies) are used to treat or prevent systemic lupus erythematosus and/or diseases, disorders or conditions associated therewith. Lupus-associated diseases, disorders, or conditions that may be treated or prevented with APO2L or IL-24 polynucleotides or polypeptides, or antagonists of the invention, include, but are not limited to, hematologic disorders (e.g., hemolytic anemia, leukopenia, lymphopenia, and thrombocytopenia), immunologic disorders (e.g., anti-DNA antibodies, and anti-Sm antibodies), rashes, photosensitivity, oral ulcers, arthritis, fever, fatigue, weight loss, serositis (e.g., pleuritus (pleuricy)), renal disorders (e.g., nephritis), neurological disorders (e.g., seizures, peripheral neuropathy, CNS related disorders), gastrointestinal disorders, Raynaud phenomenon, and pericarditis.

APO2L and/or IL-24 polypeptides, agonists, or antagonists of the invention may be used to treat diseases associated with ischemia, e.g., cardiovascular disorders, including peripheral artery disease, such as limb ischemia. They may also include stroke, vascular disease, and fulminant liver failure. In the context of an ischemic disease, the term “treating” includes any or all of preventing the growth, multiplication, or replication of the pathogen that causes the ischemic disease and ameliorating one or more symptoms of an ischemic disease.

Cardiovascular disorders include cardiovascular abnormalities, such as arterio-arterial fistula, arteriovenous fistula, cerebral arteriovenous malformations, congenital heart defects, pulmonary atresia and scimitar syndrome. Congenital heart defects include aortic coarctation, cor triatriatum, coronary vessel anomalies, crisscross heart, dextrocardia, patent ductus arteriosus, Ebstein's anomaly, Eisenmenger complex, hypoplastic left heart syndrome, levocardia, tetralogy of Fallot, transposition of great vessels, double outlet right ventricle, tricuspid atresia, persistent truncus arteriosus, and heart septal defects, such as aortopulmonary septal defect, endocardial cushion defects, Lutembacher's syndrome, trilogy of Fallot, and ventricular heart septal defects.

Cardiovascular disorders also include heart disease, such as arrhythmias, carcinoid heart disease, high cardiac output, low cardiac output, cardiac tamponade, endocarditis (including bacterial), heart aneurysm, cardiac arrest, congestive heart failure, congestive cardiomyopathy, paroxysmal dyspnea, cardiac edema, heart hypertrophy, congestive cardiomyopathy, left ventricular hypertrophy, right ventricular hypertrophy, post-infarction heart rupture, ventricular septal rupture, heart valve diseases, myocardial diseases, myocardial ischemia, pericardial effusion, pericarditis (including constrictive and tuberculous), pneumopericardium, postpericardiotomy syndrome, pulmonary heart disease, rheumatic heart disease, ventricular dysfunction, hyperemia, cardiovascular pregnancy complications, scimitar syndrome, cardiovascular syphilis, and cardiovascular tuberculosis.

Arrhythmias include sinus arrhythmia, atrial fibrillation, atrial flutter, bradycardia, extrasystole, Adams-Stokes syndrome, bundle-branch block, sinoatrial block, long QT syndrome, parasystole, Lown-Ganong-Levine syndrome, Mahaim-type pre-excitation syndrome, Wolff-Parkinson-White syndrome, sick sinus syndrome, tachycardias, and ventricular fibrillation. Tachycardias include paroxysmal tachycardia, supraventricular tachycardia, accelerated idioventricular rhythm, atrioventricular nodal reentry tachycardia, ectopic atrial tachycardia, ectopic junctional tachycardia, sinoatrial nodal reentry tachycardia, sinus tachycardia, Torsades de Pointes, and ventricular tachycardia.

Heart valve diseases include aortic valve insufficiency, aortic valve stenosis, heart murmurs, aortic valve prolapse, mitral valve prolapse, tricuspid valve prolapse, mitral valve insufficiency, mitral valve stenosis, pulmonary atresia, pulmonary valve insufficiency, pulmonary valve stenosis, tricuspid atresia, tricuspid valve insufficiency, and tricuspid valve stenosis.

Myocardial diseases include alcoholic cardiomyopathy, congestive cardiomyopathy, hypertrophic cardiomyopathy, aortic subvalvular stenosis, pulmonary subvalvular stenosis, restrictive cardiomyopathy, Chagas cardiomyopathy, endocardial fibroelastosis, endomyocardial fibrosis, Kearns syndrome, myocardial reperfusion injury, and myocarditis.

Myocardial ischemias include coronary disease, such as angina pectoris, coronary aneurysm, coronary arteriosclerosis, coronary thrombosis, coronary vasospasm, myocardial infarction and myocardial stunning.

Cardiovascular diseases also include vascular diseases such as aneurysms, angiodysplasia, angiomatosis, bacillary angiomatosis, Hippel-Lindau disease, Klippel-Trenaunay-Weber syndrome, Sturge-Weber syndrome, angioneurotic edema, aortic diseases, Takayasu's arteritis, aortitis, Leriche's syndrome, arterial occlusive diseases, arteritis, enarteritis, polyarteritis nodosa, cerebrovascular disorders, diabetic angiopathies, diabetic retinopathy, embolisms, thrombosis, erythromelalgia, hemorrhoids, hepatic veno-occlusive disease, hypertension, hypotension, ischemia, peripheral vascular diseases, phlebitis, pulmonary veno-occlusive disease, Raynaud's disease, CREST syndrome, retinal vein occlusion, Scimitar syndrome, superior vena cava syndrome, telangiectasia, ataxia telangiectasia, hereditary hemorrhagic telangiectasia, varicocele, varicose veins, varicose ulcer, vasculitis, and venous insufficiency.

Aneurysms include dissecting aneurysms, false aneurysms, infected aneurysms, ruptured aneurysms, aortic aneurysms, cerebral aneurysms, coronary aneurysms, heart aneurysms, and iliac aneurysms.

Arterial occlusive diseases include arteriosclerosis, intermittent claudication, carotid stenosis, fibromuscular dysplasias, mesenteric vascular occlusion, Moyamoya disease, renal artery obstruction, retinal artery occlusion, and thromboangitis obliterans.

Cerebrovascular disorders include carotid artery diseases, cerebral amyloid angiopathy, cerebral aneurysm, cerebral anoxia, cerebral arteriosclerosis, cerebral arteriovenous malformation, cerebral artery diseases, cerebral embolism and thrombosis, carotid artery thrombosis, sinus thrombosis, Wallenberg's syndrome, cerebral hemorrhage, epidural hematoma, subdural hematoma, subarachnoid hemorrhage, cerebral infarction, cerebral ischemia (including transient), subclavian steal syndrome, periventricular leukomalacia, vascular headache, cluster headache, migraine, and vertebrobasilar insufficiency.

Embolisms include air embolisms, amniotic fluid embolisms, cholesterol embolisms, blue toe syndrome, fat embolisms, pulmonary embolisms, and thromboembolisms. Thromboses include coronary thrombosis, hepatic vein thrombosis, retinal vein occlusion, carotid artery thrombosis, sinus thrombosis, Wallenberg's syndrome, and thrombophlebitis.

Ischemia includes cerebral ischemia, ischemic colitis, compartment syndromes, anterior compartment syndrome, myocardial ischemia, reperfusion injuries, and peripheral limb ischemia. Vasculitis includes aortitis, arteritis, Behcet's syndrome, Churg-Strauss syndrome, mucocutaneous lymph node syndrome, thromboangiitis obliterans, hypersensitivity vasculitis, Schoenlein-Henoch purpura, allergic cutaneous vasculitis, and Wegener's granulomatosis.

The present invention further provides for treatment of diseases or disorders associated with neovascularization by administration of the APO2L or IL-24 polynucleotides and/or polypeptides of the invention (including APO2L or IL-24 agonists and/or antagonists). Malignant and metastatic conditions which can be treated with the polynucleotides and polypeptides of the invention include, but are not limited to those malignancies, solid tumors, and cancers described herein and otherwise known in the art (for a review of such disorders, see Fishman et al., Medicine, 2d ed., J. B. Lippincott Co. (1985)).

Additionally, ocular disorders associated with neovascularization which can be treated with the APO2L or IL-24 polynucleotides and polypeptides of the present invention (including APO2L or IL-24 agonists and APO2L or IL-24 antagonists) include, but are not limited to neovascular glaucoma, diabetic retinopathy, retinoblastoma, retrolental fibroplasia, uveitis, retinopathy of prematurity, macular degeneration, corneal graft neovascularization, as well as other eye inflammatory diseases, ocular tumors, and diseases associated with choroidal or iris neovascularization. See, e.g., reviews by Waltman et al., Am. J. Ophthal., 85:704-710 (1978) and Gartner et al., Surv. Ophthal., 22:291-312 (1978).

Additionally, disorders which can be treated with the APO2L or IL-24 polynucleotides and polypeptides of the present invention (including APO2L or IL-24 agonists and APO2L or IL-24 antagonists) include, but are not limited to, hemangioma, arthritis, psoriasis, angiofibroma, atherosclerotic plaques, delayed wound healing, granulations, hemophilic joints, hypertrophic scars, nonunion fractures, Osler-Weber syndrome, pyogenic granuloma, scleroderrna, trachoma, and vascular adhesions.

Polynucleotides and/or polypeptides of the invention, and/or agonists and/or antagonists thereof, are useful in the diagnosis and treatment or prevention of a wide range of diseases and/or conditions. Such diseases and conditions include, but are not limited to, cancer (e.g., immune cell related cancers, breast cancer, prostate cancer, ovarian cancer, follicular lymphoma, cancer associated with mutation or alteration of p53, brain tumor, bladder cancer, uterocervical cancer, colon cancer, colorectal cancer, non-small cell carcinoma of the lung, small cell carcinoma of the lung, stomach cancer, etc.), lymphoproliferative disorders (e.g., lymphadenopathy), microbial (e.g., viral, bacterial, etc.) infection (e.g., HIV-1 infection, HIV-2 infection, herpes virus infection (including, but not limited to, HSV-1, HSV-2, CMV, VZV, HHV-6, HHV-7, EBV), adenovirus infection, poxvirus infection, human papilloma virus infection, hepatitis infection (e.g., HAV, HBV, HCV, etc.), Helicobacterpylori infection, invasive Staphylococci, etc.), parasitic infection, nephritis, bone disease (e.g., osteoporosis), atherosclerosis, pain, cardiovascular disorders (e.g., neovascularization, hypovascularization or reduced circulation (e.g., ischemic disease (e.g., myocardial infarction, stroke, etc.)), AIDS, allergy, inflammation, neurodegenerative disease (e.g., Alzheimer's disease, Parkinson's disease, amyotrophic lateral sclerosis, pigmentary retinitis, cerebellar degeneration, etc.), graft rejection (acute and chronic), graft vs. host disease, diseases due to osteomyelodysplasia (e.g., aplastic anemia, etc.), joint tissue destruction in rheumatism, liver disease (e.g., acute and chronic hepatitis, liver injury, and cirrhosis), autoimmune disease (e.g., multiple sclerosis, rheumatoid arthritis, systemic lupus erythematosus, immune complex glomerulonephritis, autoimmune diabetes, autoimmune thrombocytopenic purpura, Grave's disease, Hashimoto's thyroiditis, etc.), cardiomyopathy (e.g., dilated cardiomyopathy), diabetes, diabetic complications (e.g., diabetic nephropathy, diabetic neuropathy, diabetic retinopathy), influenza, asthma, psoriasis, glomerulonephritis, septic shock, and ulcerative colitis.

Polynucleotides and/or polypeptides of the invention and/or agonists and/or antagonists thereof are useful in promoting angiogenesis, wound healing (e.g., wounds, bums, and bone fractures).

Polynucleotides and/or polypeptides of the invention and/or agonists and/or antagonists thereof are also useful as an adjuvant to enhance immune responsiveness to specific antigen and/or anti-viral immune responses.

More generally, polynucleotides and/or polypeptides of the invention and/or agonists and/or antagonists thereof are useful in regulating (i.e., elevating or reducing) the immune response. For example, polynucleotides and/or polypeptides of the invention may be useful in preparation or recovery from surgery, trauma, radiation therapy, chemotherapy, and transplantation, or may be used to boost immune response and/or recovery in the elderly and immunocompromised individuals. Alternatively, polynucleotides and/or polypeptides of the invention and/or agonists and/or antagonists thereof are useful as immunosuppressive agents, for example in the treatment or prevention of autoimmune disorders. In specific embodiments, polynucleotides and/or polypeptides of the invention are used to treat or prevent chronic inflammatory, allergic or autoimmune conditions, such as those described herein or are otherwise known in the art.

The uses of the APO2L or IL-24 polypeptides, include, but are not limited to, the treatment or prevention of viral hepatitis, herpes viral infections, allergic reactions, adult respiratory distress syndrome, neoplasia, anaphylaxis, allergic astima, allergen rhinitis, drug allergies (e.g., to penicillin, cephalosporins), primary central nervous system lymphoma (PCNSL), glioblastoma, chronic lymphocytic leukemia (CLL), lymphadenopathy, autoimmune disease, graft versus host disease, rheumatoid arthritis, osteoarthritis, Graves' disease, acute lymphoblastic leukemia (ALL), lymphomas (Hodgkin's disease and non-Hodgkin's lymphoma (NHL)), ophthalmopathy, uveoretinitis, the autoimmune phase of Type 1 diabetes, myasthenia gravis, glomerulonephritis, autoimmune hepatological disorder, autoimmune inflammatory bowel disease, and Crohn's disease. In addition, the APO2L or IL-24 polypeptides of the present invention may be employed to inhibit neoplasia, such as tumor cell growth. The combination of APO2L or IL-24 protein with immunotherapeutic agents such as IL-2 or IL-12 may result in synergistic or additive effects that would be useful for the treatment of established cancers.

### Antibodies

APO2L or IL-24 -protein specific antibodies for use in the present invention can be raised against the intact APO2L or IL-24 protein or an antigenic polypeptide fragment thereof. The protein or fragment may be presented with or without a carrier protein, such as an albumin, to an animal system (such as rabbit or mouse); in general the polypeptide fragments are sufficiently immunogenic to produce a satisfactory immune response without a carrier if they are at least about 25 amino acids in length.

Antibodies of the invention include polyclonal and monoclonal antibody preparations, as well as preparations including hybrid antibodies, altered antibodies, chimeric antibodies and, humanized antibodies, as well as: hybrid (chimeric) antibody molecules (see, for example, Winter et al., Nature 349:293-299 (1991)); and U.S. Pat. No. 4,816,567); F(ab′)₂ and F(ab) fragments; Fv molecules (noncovalent heterodimers, see, for example, Inbar et al., Proc. Natl. Acad. Sci. 69:2659-2662 (1972)); and Ehrlich et al. (1980) Biochem 19:4091-4096); single-chain Fv molecules (sFv) (see, e.g., Huston et al., Proc. Natl. Acad. Sci. 85:5879-5883 (1980)); dimeric and trimeric antibody fragment constructs; minibodies (see, e.g., Pack et al., Biochem. 31:1579-1584 (1992); Cumber et al., J. Immunology 149B:120-126 (1992)); humanized antibody molecules (see, e.g., Riechmann et al., Nature 332:323-327 (1988); Verhoeyan et al., Science 239:1534-1536 (1988)); and any functional fragments obtained from such molecules, wherein such fragments retain specific binding.

Methods of making monoclonal and polyclonal antibodies are known in the art. Monoclonal antibodies are generally antibodies having a homogeneous antibody population. The term is not limited regarding the species or source of the antibody, nor is it intended to be limited by the manner in which it is made. The term encompasses whole immunoglobulins. Polyclonal antibodies are generated by immunizing a suitable animal, such as a mouse, rat, rabbit, sheep or goat, with an antigen of interest, such as a stem cell transformed with a gene encoding an antigen. In order to enhance immunogenicity, the antigen can be linked to a carrier prior to immunization. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Furthermore, the antigen may be conjugated to a bacterial toxoid, such as toxoid from diphtheria, tetanus, cholera, etc., in order to enhance the immunogenicity thereof.

In addition, techniques developed for the production of chimeric antibodies (Morrison et al., Proc. Natl. Acad. Sci., 81:851-855 (1984); Neuberger et al., Nature, 312:604-608 (1984); Takeda et al., Nature, 314:452-454 (1985)) by splicing genes from a mouse antibody molecule of appropriate antigen specificity together with genes from a human antibody molecule of appropriate biological activity can be used. Chimeric antibodies, i.e., antibdies in which different portions are derived from different animal species, such as those having a variable region derived from a murine monoclonal antibody and a human immunoglobulin constant region, for example, humanized antibodies, and insertion/deletions relating to cdr and framework regions, re suitable for use in the invention.

The invention also includes humanized antibodies, i.e., those with mostly human immunoglobulin sequences. Humanized antibodies of the invention generally refer to nonhuman immunoglobulins that have been modified to incorporate portions of human sequences. A humanized antibody may include a human antibody that contains entirely human immunoglobulin sequences.

The antibodies of the invention may be prepared by any of a variety of methods. For example, cells expressing the APO2L or IL-24 protein or an antigenic fragment thereof can be administered to an animal in order to induce the production of sera containing polyclonal antibodies. A preparation of APO2L or IL-24 protein can be prepared and purified to render it substantially free of natural contaminants, and the preparation introduced into an animal in order to produce polyclonal antisera with specific binding activity.

Antibodies of the invention specifically bind to their respective antigen(s); they may display high avidity and/or high affinity to a specific polypeptide, or more accurately, to an epitope of an antigen. Antibodies of the invention may bind to one epitope, or to more than one epitope. They may display different affinities and/or avidities to different epitopes on one or more molecules. When an antibody binds more strongly to one epitope than to another, adjusting the binding conditions can, in some instances, result in antibody binding almost exclusively to the specific epitope and not to any other epitopes on the same polypeptide, and not to a polypeptide that does not comprise the epitope.

The invention also provides monoclonal antibodies and APO2L or IL-24 protein binding fragments thereof. Monoclonal antibodies of the invention can be prepared using hybridoma technology, for example, Kohler et al., Nature, 256:495 (1975); Kohler et al., Eur. J. Immunol., 6:511 (1976); Kohler et. al., Eur. J. Immunol., 6:292 (1976); Hammerling et al., in: Monoclonal Antibodies and T-Cell Hybridoinas, Elsevier, N.Y., (1981) pp. 563-681). In general, such procedures involve immunizing an animal (e.g., a mouse) with an APO2L or IL-24 protein antigen or with an APO2L or IL-24 protein-expressing cell. Suitable cells can be recognized by their capacity to bind anti-APO2L or IL-24 protein antibody. Such cells may be cultured in any suitable tissue culture medium; e.g., in Earle's modified Eagle's medium supplemented with 10% fetal bovine serum (inactivated at about 56° C.), and supplemented with about 10 grams/liter of nonessential amino acids, about 1,000 U/ml of penicillin, and about 100 μg/ml of streptomycin. The splenocytes of such mice are extracted and fused with a suitable myeloma cell line. Any suitable myeloma cell line may be employed in accordance with the present invention; e.g., the parent myeloma cell line (SP20), available from the American Type Culture Collection, Manassas, Va. After fusion, the resulting hybridoma cells are selectively maintained in HAT medium, and then cloned by limiting dilution as described by Wands et al., Gastroenterology, 80:225-232 (1981).

APO2L and IL-24 Protein Antigens

Alternatively, antibodies capable of binding to the APO2L or IL-24 protein antigen may be produced in a two-step procedure through the use of anti-idiotypic antibodies. Such a method makes use of the fact that antibodies are themselves antigens, and that, therefore, it is possible to obtain an antibody which binds to a second antibody. In accordance with this method, APO2L or IL-24-protein specific antibodies are used to immunize an animal, e.g., a mouse. The splenocytes of such an animal are then used to produce hybridoma cells, and the hybridoma cells are screened to identify clones which produce an antibody whose ability to bind to the APO2L or IL-24 protein-specific antibody can be blocked by the APO2L or IL-24 protein antigen. Such antibodies comprise anti-idiotypic antibodies to the APO2L or IL-24 protein-specific antibody and can be used to imnmunize an animal to induce formation of further APO2L or IL-24 protein-specific antibodies.

It will be appreciated that Fab and F(ab′)2 and other fragments of the antibodies of the present invention may be used according to the methods disclosed herein. Such fragments are typically produced by proteolytic cleavage, using enzymes such as papain (to produce Fab fragments) or pepsin (to produce F(ab′)2 fragments). Alternatively, APO2L or IL-24 protein-binding fragments can be produced through the application of recombinant DNA technology or through synthetic chemistry. Humanized chimeric monoclonal antibodies are suitable for in vivo use of anti-APO2L or IL-24 in humans. Such humanized antibodies can be produced using genetic constructs derived from hybridoma cells producing the monoclonal antibodies described above. Methods for producing chimeric antibodies are known in the art. See, for review, Morrison, Science, 229:1202 (1985); Oi et al., BioTechniques, 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature, 312:643 (1984); Neuberger et al., Nature, 314:268 (1985).

### Diagnosis

This invention is also related to the use of the genes of the present invention as part of a diagnostic assay for detecting diseases or susceptibility to diseases related to the presence of mutations in the nucleic acid sequences encoding the polypeptide of the present invention. Individuals carrying mutations in a gene of the present invention may be detected at the DNA level by a variety of techniques. Nucleic acids for diagnosis may be obtained from a patient's cells, such as from blood, urine, saliva, tissue biopsy, and autopsy material. The genomic DNA may be used directly for detection or may be amplified enzymatically by using PCR, for example, as described by Saiki et al., Nature, 324: 163-166 (1986), prior to analysis. RNA or cDNA may also be used for the same purpose. As an example, PCR primers complementary to the nucleic acid encoding a polypeptide of the present invention can be used to identify and analyze mutations. For example, deletions and insertions can be detected by a change in size of the amplified product in comparison to the normal genotype. Point mutations can be identified by hybridizing amplified DNA to radiolabeled RNA or alternatively, radiolabeled antisense DNA sequences. Perfectly matched sequences can be distinguished from mismatched duplexes by RNase A digestion or by differences in melting temperatures.

Genetic testing based on DNA sequence differences may be achieved by detecting alterations in electrophoretic mobility of DNA fragments in gels run with or without denaturing agents. Small sequence deletions and insertions can be visualized by high resolution gel electrophoresis. DNA fragments of different sequences may be distinguished on denaturing formamide gradient gels in which the mobilities of different DNA fragments are retarded in the gel at different positions according to their specific melting or partial melting temperatures, for example, as described by Myers et al., Science, 230:1242 (1985).

Sequence changes at specific locations may also be revealed by nuclease protection assays, such as RNase and S 1 protection or the chemical cleavage method as shown in Cotton et al., Proc. Natl. Acad. Sci., USA, 85:4397-4401 (1985). Thus, the detection of a specific DNA sequence may be achieved by methods such as hybridization, RNase protection, chemical cleavage, direct DNA sequencing or the use of restriction enzymes, (e.g., Restriction Fragment Length Polymorphisms (RFLP)) and Southern blotting of genomic DNA. In addition to more conventional gel-electrophoresis and DNA sequencing, mutations can also be detected by in situ analysis.

The present invention also relates to a diagnostic assay for detecting altered levels of APO2L or IL-24 proteins in various tissues. An over-expression of these proteins compared to normal control tissue samples may detect the presence of abnormal cellular proliferation, for example, a tumor. Assays used to detect protein levels in a host-derived sample are well-known to those of skill in the art and include radioimmunoassays, competitive-binding assays, Western Blot analysis, ELISA assays, “sandwich” assays, and other assays for the expression levels of the genes encoding the APO2L or IL-24 proteins known in the art. Expression can be assayed by qualitatively or quantitatively measuring or estimating the level of APO2L or IL-24 protein, or the level of mRNA encoding APO2L or IL-24 protein, in a biological sample. Assays may be performed directly, for example, by determining or estimating absolute protein level or mRNA level, or relatively, by comparing the APO2L or IL-24 protein or mRNA to a second biological sample. In performing these assays, the APO2L or IL-24 protein or mRNA level in the first biological sample is measured or estimated and compared to a standard APO2L or IL-24 protein level or mRNA level; suitable standards include second biological samples obtained from an individual not having the disorder of interest. Standards may be obtained by averaging levels of APO2L or IL-24 in a population of individuals not having a disorder related to APO2L or IL-24 expression. As will be appreciated in the art, once a standard APO2L or IL-24 protein level or mRNA level is known, it can be used repeatedly as a standard for comparison.

An ELISA assay, for example, as described by Coligan, et al., Current Protocols in Immunology, 1(2), Chap. 6, (1991), utilizes an antibody prepared with specificity to a polypeptide antigen of the present invention. In addition, a reporter antibody is prepared against the monoclonal antibody. To the reporter antibody is attached a detectable reagent such as a radioactive tag, a fluorescent tag, or an enzymatic tag, e.g., a horseradish peroxidase. A sample is removed from a host and incubated on a solid support, e.g. a polystyrene dish, that binds the proteins in the sample. Any free protein binding sites on the dish are then covered by incubating with a non-specific protein, e.g., bovine serum albumin. Next, the specific antibody, e.g., a monoclonal antibody, is incubated in the dish, during which time the antibody attaches to any polypeptides of the present invention attached to the polystyrene dish. All unbound monoclonal antibody is washed out with buffer. The reporter antibody, i.e., one linked to horseradish peroxidase is placed in the dish, resulting in the binding of the reporter antibody to any antibody bound to the protein of interest; unattached reporter antibody is then removed. Substrate, e.g., peroxidase, is then added to the dish, and the amount of signal produced color, e.g., developed in a given time period provides a measurement of the amount of a polypeptide of the present invention present in a given volume of patient sample when compared against a standard.

A competition assay may be employed wherein antibodies specific to a polypeptide of the present invention are attached to a solid support, and labeled APO2L or IL-24, along with a sample derived from the host, are passed over the solid support. The label can be detected and quantified, for example, by liquid scintillation chromatography, and the measurement can be correlated to the quantity of the polypeptide of interest present in the sample. A “sandwich” assay, similar to an ELISA assay, may be employed, wherein a polypeptide of the present invention is passed over a solid support and binds to antibody modules attached to the solid support. A second antibody is then bound to the polypeptide of interest. A third antibody, which is labeled and specific to the second antibody is then passed over the solid support and binds to the second antibody. The amount of antibody binding can be quantified; it correlates with the amount of the polypeptide of interest.

Biological samples of the invention can include any biological sample obtained from a subject, body fluid, cell line, tissue culture, or other source which contains APO2L or IL-24 protein or mRNA. As indicated, biological samples include body fluids (such as sera, plasma, urine, synovial fluid, and spinal fluid) which contain free APO2L or IL-24 protein, ovarian or renal system tissue, and other tissue sources found to express complete or mature APO2L or IL-24 polypeptide or an APO2L or IL-24 receptor. Methods for obtaining tissue biopsies and body fluids from mammals are well known in the art. Where the biological sample is to include mRNA, a tissue biopsy may provide the source.

Total cellular RNA can be isolated from a biological sample using any suitable technique such as the single-step guanidinium-thiocyanate-phenol-chloroform method described in Chomczynski and Sacchi, Anal. Biochem., 162:156-159 (1987). Levels of mRNA encoding the APO2L or IL-24 protein are then assayed using any appropriate method. These include Northern blot analysis, S1 nuclease mapping, PCR, reverse transcription in combination with PCR (RT-PCR), and reverse transcription in combination with the ligase chain reaction (RT-LCR).

Assaying APO2L or IL-24 protein levels in a biological sample can be performed using antibody-based techniques. For example, APO2L or IL-24 protein expression in tissues can be studied with classical immunohistological methods, for example, Jalkanen, M., et al., J. Cell. Biol., 101:976-985 (1985); Jalkanen, M., et al., J. Cell. Biol., 105:3087-3096 (1987). Other antibody-based methods useful for detecting APO2L or IL-24 protein gene expression include immunoassays, such as the enzyme linked immunosorbent assay (ELISA) and the radioimmunoassay (RIA). Suitable antibody assay labels are known in the art and include enzyme labels, such as glucose oxidase, radioisotopes, and fluorescent labels, such as fluorescein and rhodamine, and biotin.

In addition to assaying APO2L or IL-24 protein levels in a biological sample obtained from an individual, APO2L or IL-24 protein can also be detected ill vivo by imaging. Antibody labels or markers for in vivo imaging of APO2L or IL-24 protein include those detectable by X-radiography, NMR, or ESR. For X-radiography, suitable labels include radioisotopes such as barium or cesium, which emit detectable radiation but are not overtly harmful to a subject. Suitable markers for NMR and ESR include those with a detectable characteristic spin, such as deuterium, which may be incorporated into the antibody by labeling of nutrients for the relevant hybridoma.

An APO2L or IL-24 protein-specific antibody or antibody fragment which has been labeled with an appropriate detectable imaging moiety, such as a radioisotope, a radio-opaque substance, or a material detectable by nuclear magnetic resonance, is introduced, for example, parenterally, subcutaneously or intraperitoneally, into the subject to be examined for an immune system disorder. It will be understood in the art that the size of the subject and the imaging system used will determine the quantity of imaging moiety needed to produce diagnostic images. The labeled antibody or antibody fragment will then preferentially accumulate at the location of cells which contain APO2L or IL-24 protein. In vivo tumor imaging is described in Burchiel et al., ed., Chapter 13, Tumor Imaging: The Radiochemical Detection of Cancer, Masson Publishing Inc. (1982).

### Formulations

The APO2L or IL-24 polypeptide compositions will be formulated and dosed in a fashion consistent with good medical practice, taking into account the clinical condition of the individual subject, the site of delivery of the APO2L or IL-24 polypeptide composition, the method of administration, the scheduling of administration, and other factors known to practitioners. The effective amount of APO2L or IL-24 polypeptide for purposes herein is thus determined by such considerations.

The polypeptides, agonists, and antagonists of the present invention may be employed in combination with a suitable pharmaceutical carrier to comprise a pharmaceutical composition for parenteral administration. Such compositions comprise a therapeutically effective amount of the polypeptide, agonist, or antagonist and a pharmaceutically acceptable carrier or excipient. Such a carrier includes, but is not limited to, saline, buffered saline, dextrose, water, glycerol, ethanol, and combinations thereof. The formulation should suit the mode of administration.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the polypeptides, agonists and antagonists of the present invention may be employed in conjunction with other therapeutic compounds.

The pharmaceutical compositions may be administered in a convenient mainer such as by the oral, topical, intravenous, intraperitoneal, intramuscular, subcutaneous, intranasal, or intradermal routes. The pharmaceutical compositions are administered in an amount which is effective for treating and/or prophylaxis of the specific indication. In general, they are administered in an amount of at least about 10 micrograms/kg body weight and in most cases they will be administered in an amount not in excess of about 8 milligrams/kg body weight per day.

The polypeptides of the invention, and agonist and antagonist compounds which are polypeptides, may also be employed in accordance with the present invention by expression of such polypeptides in vivo, i.e., gene therapy. Thus, for example, cells may be engineered with a polynucleotide (DNA or RNA) encoding for the polypeptide ex vivo; the engineered cells are then provided to a patient. Such methods are well-known in the art. For example, cells may be engineered by procedures known in the art by use of a retroviral particle containing RNA encoding for the polypeptide of the present invention.

Similarly, cells may be engineered in vivo for expressing the polypeptide in vivo, for example, by procedures known in the art. As known in the art, a cell producing a retroviral particle containing RNA encoding the polypeptide of the present invention may be administered to a patient for the purpose of engineering cells in vivo and expressing the polypeptide in vivo. These and other methods for administering a polypeptide of the present invention by similar methods should be apparent to those skilled in the art from the teachings of the present invention. For example, the expression vehicle for engineering cells may be other than a retroviral particle, for example, an adenovirus, which may be used to engineer cells in vivo after combination with a suitable delivery vehicle.

Retroviruses from which the retroviral plasmid vectors hereinabove mentioned may be derived include, but are not limited to, Moloney Murine Leukemia virus, spleen necrosis virus, retroviruses such as Rous sarcoma virus, Harvey sarcoma virus, avian leukosis virus, gibbon ape leukemia virus, human immunodeficiency virus, adenovirus, myeloproliferative sarcoma virus, and mammary tumor virus. In one embodiment, the retroviral plasmid vector is derived from Moloney murine leukemia virus.

The nucleic acid sequence encoding the polypeptide of the present invention is under the control of a suitable promoter. Vectors of the invention include one or more promoters. Suitable promoters which may be employed include, but are not limited to, the retroviral LTR; the SV40 promoter; and the human cytomegalovirus (CMV) promoter described in Miller, et al., Biotechniques, Vol. 7, No. 9, 980-990 (1989), or any other homologous or heterologous promoter, for example, cellular promoters such as eukaryotic cellular promoters including, but not limited to, the histone, pol III, and β-actin promoters. Other viral promoters which may be employed include, but are not limited to, adenovirus promoters, e.g., the adenoviral major late promoter; thymidine kinase (TK) promoters; and B19 parvovirus promoters.

Suitable promoters include the respiratory syncytial virus (RSV) promoter; inducible promoters, such as the MMT promoter, the metallothionein promoter; heat shock promoters; the albumin promoter; the ApoAl promoter; human globin promoters; viral thymidine kinase promoters, such as the Herpes Simplex thymidine kinase promoter; retroviral LTRs (including the modified retroviral LTRs hereinabove described); the beta-actin promoter; and human growth hormone promoters. The promoter also may be the native promoter which controls the gene encoding the polypeptide. The selection of a suitable promoter will be apparent to those skilled in the art from the teachings contained herein.

A retroviral plasmid vector can be employed to transduce packaging cell lines to form producer cell lines. Examples of packaging cells which may be transfected include, but are not limited to, the PE501, PA317, -2, -AM, PA12, T19-14X, VT-19-17-H2, CRE, CRIP, GP+E-86, GP+envAm12, and DAN cell lines as described in Miller, Human Gene Therapy, 1:5-14 (1990). The vector may transduce the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host.

The producer cell line generates infectious retroviral vector particles which include the nucleic acid sequence(s) encoding the polypeptides. Such retroviral vector particles then may be employed to transduce eukaryotic cells, either in vitro or in vivo. The transduced eukaryotic cells will express the nucleic acid sequence(s) encoding the polypeptide. Eukaryotic cells which may be transduced include, but are not limited to, embryonic stem cells, embryonic carcinoma cells, as well as hematopoietic stem cells, hepatocytes, fibroblasts, myoblasts, keratinocytes, endothelial cells, and bronchial epithelial cells.

### APO2L or IL-24 “Knock-outs” and Homologous Recombination

Endogenous gene expression can be reduced by inactivating or “knocking out” a gene of interest and/or its promoter using targeted homologous recombination. (e.g., see Smithies et al., Nature, 317:230-234 (1985); Thomas & Capecchi, Cell, 51:503-512 (1987); Thompson et al., Cell, 5:313-321 (1989); each of which is incorporated by reference herein in its entirety). For example, a mutant, non-functional polynucleotide of the invention (or a completely unrelated DNA sequence) flanked by DNA homologous to the endogenous polynucleotide sequence (either the coding regions or regulatory regions of the gene) can be used, with or without a selectable marker and/or a negative selectable marker, to transfect cells that express polypeptides of the invention in vivo. In another embodiment, techniques known in the art are used to generate knockouts in cells that contain, but do not express, the gene of interest. Insertion of the DNA construct, via targeted homologous recombination, results in inactivation of the targeted gene. Such approaches are particularly suited in research and agricultural fields where modifications to embryonic stem cells can be used to generate animal offspring with an inactive targeted gene (see, e.g., Thomas & Capecchi 1987 and Thompson 1989, supra). However, this approach can be routinely adapted for use in humans provided the recombinant DNA constructs are directly administered or targeted to the required site in vivo using appropriate viral vectors that will be apparent to those of skill in the art.

In further embodiments of the invention, cells that are genetically engineered to express the polypeptides of the invention, or alternatively, that are genetically engineered not to express the polypeptides of the invention (e.g., knockouts) are administered to a patient in vivo. Such cells may be obtained from the patient (i.e., animal, including human) or an MHC compatible donor and can include, but are not limited to fibroblasts, bone marrow cells, blood cells (e.g., lymphocytes), adipocytes, muscle cells, endothelial cells, etc. The cells are genetically engineered in vitro using recombinant DNA techniques to introduce the coding sequence of polypeptides of the invention into the cells, or alternatively, to disrupt the coding sequence and/or endogenous regulatory sequence associated with the polypeptides of the invention, e.g., by transduction (using viral vectors, and/or vectors that integrate the transgene into the cell genome) or transfection procedures, including, but not limited to, the use of plasmids, cosmids, YACs, naked DNA, electroporation, liposomes, etc. The coding sequence of the polypeptides of the invention can be placed under the control of a strong constitutive or inducible promoter or promoter/enhancer to achieve expression, and secretion, of the polypeptides of the invention. The engineered cells which express and secrete the polypeptides of the invention can be introduced into the patient systemically, e.g., in the circulation, or intraperitoneally. Alternatively, the cells can be incorporated into a matrix and implanted in the body, e.g., genetically engineered fibroblasts can be implanted as part of a skin graft; genetically engineered endothelial cells can be implanted as part of a lymphatic or vascular graft. (See, for example, Anderson et al. U.S. Pat. No. 5,399,349; and Mulligan & Wilson, U.S. Pat. No. 5,460,959, each of which is incorporated by reference herein in its entirety).

When the cells to be administered are non-autologous or non-MHC compatible cells, they can be administered using well known techniques which prevent the development of a host immune response against the introduced cells. For example, the cells may be introduced in an encapsulated form which, while allowing for an exchange of components with the immediate extracellular environment, does not allow the introduced cells to be recognized by the host immune system.

### Transgenic Non-Human Animals

The polypeptides of the invention can also be expressed in transgenic animals. Animals of any species, including, but not limited to, mice, rats, rabbits, hamsters, guinea pigs, pigs, micro-pigs, goats, sheep, cows, and non-human primates, e.g., baboons, monkeys, and chimpanzees may be used to generate transgenic animals. In a specific embodiment, techniques described herein or otherwise known in the art, are used to express polypeptides of the invention in humans, as part of a gene therapy protocol.

Any technique known in the art may be used to introduce the transgene (i.e., polynucleotides of the invention) into animals to produce a founder lines of transgenic animals. Such techniques include, but are not limited to, pronuclear microinjection (Paterson et al., Appl. Microbiol. Biotechnol. 40:691-698 (1994); Carver et al., Biotechnology (NY) 11: 1263-1270 (1993); Wright et al., Biotechnology (NY) 9:830-834 (1991); and Hoppe et al., U.S. Pat. No. 4,873,191 (1989)); retrovirus mediated gene transfer into germ lines (Van der Putten et al., Proc. Natl. Acad. Sci., USA 82:6148-6152 (1985)), blastocysts or embryos; gene targeting in embryonic stem cells (Thompson et al., Cell 56:313-321 (1989)); electroporation of cells or embryos (Lo, Mol. Cell. Biol. 3:1803-1814 (1983)); introduction of the polynucleotides of the invention using a gene gun (see, e.g., Ulmer et al., Science 259:1745 (1993); introducing nucleic acid constructs into embryonic pluripotent stem cells and transferring the stem cells back into the blastocyst; and sperm-mediated gene transfer (Lavitrano et al., Cell 57:717-723 (1989); etc. For a review of such techniques, see Gordon, Intl. Rev. Cytol. 115:171-229 (1989), which is incorporated by reference herein in its entirety. Further, the contents of each of the documents recited in this paragraph is herein incorporated by reference in its entirety. See also, U.S. Pat. No. 5,464,764 (Capecchi et al., Positive-Negative Selection Methods and Vectors); U.S. Pat. No. 5,631,153 (Capecchi et al., Cells and Non-Human Organisms Containing Predetermined Genomic Modifications and Positive-Negative Selection Methods and Vectors for Making Same); U.S. Pat. No. 4,736,866 (Leder et al., Transgenic Non-Human Animals); and U.S. Pat. No. 4,873,191 (Wagner et al., Genetic Transformation of Zygotes); each of which is hereby incorporated by reference in its entirety. Any technique known in the art may be used to produce transgenic clones containing polynucleotides of the invention, for example, nuclear transfer into enucleated oocytes of nuclei from cultured embryonic, fetal, or adult cells induced to quiescence (Campbell et al., Nature 380:64-66 (1996); Wilmut et al., Nature 385:810-813 (1997)), each of which is herein incorporated by reference in its entirety).

The present invention provides for transgenic animals that carry the transgene in all their cells, as well as animals which carry the transgene in some, but not all their cells, i.e., mosaic or chimeric animals. The transgene may be integrated as a single transgene or as multiple copies such as in concatamers, e.g., head-to-head tandems or head-to-tail tandems. The transgene may also be selectively introduced into and activated in a particular cell type by following, for example, the teaching of Lasko et al. (Proc. Natl. Acad. Sci. USA 89:6232-6236 (1992)). The regulatory sequences required for such a cell-type specific activation will depend upon the particular cell type of interest, and will be apparent to those of skill in the art. It may be desired that the polynucleotide transgene be integrated into the chromosomal site of the endogenous gene, gene targeting is then suitable. Briefly, when stich a technique is to be utilized, vectors containing some nucleotide sequences homologous to the endogenous gene are designed for the purpose of integrating, via homologous recombination with chromosomal sequences, into and disrupting the function of the nucleotide sequence of the endogenous gene. The transgene may also be selectively introduced into a particular cell type, thus inactivating the endogenous gene in only that cell type, by following, for example, the teaching of Gu et al. (Science 265:103-106 (1994)). The regulatory sequences required for such a cell-type specific inactivation will depend upon the particular cell type of interest, and will be apparent to those of skill in the art.

Once transgenic animals have been generated, the expression of the recombinant gene may be assayed utilizing standard techniques. Initial screening may be accomplished by Southern blot analysis or PCR techniques to analyze animal tissues to verify that integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, and reverse transcriptase-PCR (rt-PCR). Samples of transgenic gene-expressing tissue may also be evaluated immunocytochemically or immunohistochemically using antibodies specific for the transgene product.

Once the founder animals are produced, they may be bred, inbred, outbred, or crossbred to produce colonies of the particular animal. Examples of such breeding strategies include, but are not limited to outbreeding of founder animals with more than one integration site in order to establish separate lines; inbreeding of separate lines in order to produce compound transgenics that express the transgene at higher levels because of the effects of additive expression of each transgene; crossing of heterozygous transgenic animals to produce animals homozygous for a given integration site in order to both augment expression and eliminate the need for screening of animals by DNA analysis; crossing of separate homozygous lines to produce compound heterozygous or homozygous lines; and breeding to place the transgene on a distinct background that is appropriate for an experimental model of interest.

Transgenic and “knock-out” animals of the invention have uses which include, but are not limited to, animal model systems useful in elaborating the biological function of APO2L or IL-24 polypeptides, studying conditions and/or disorders associated with aberrant APO2L or IL-24 expression, and in screening for compounds effective in ameliorating such conditions and/or disorders.

### Kits

The present invention provides kits that can be used in the above methods. In one embodiment, a kit comprises an antibody of the invention, e.g., a purified antibody, in one or more containers. In a specific embodiment, the kits of the present invention contain a substantially isolated polypeptide comprising an epitope which is specifically immunoreactive with an antibody included in the kit. The kits of the present invention may also comprise a control antibody which does not react with the polypeptide of interest.

In another embodiment, the kits of the present invention comprise a means for detecting the binding of an antibody to a polypeptide of interest (e.g., the antibody may be conjugated to a detectable substrate such as a fluorescent compound, an enzymatic substrate, a radioactive compound or a luminescent compound, or a second antibody which recognizes the first antibody may be conjugated to a detectable substrate).

In another embodiment of the present invention, the kit is a diagnostic kit for use in screening serum containing antibodies specific against proliferative and/or cancerous polynucleotides and polypeptides. Such a kit may include a control antibody that does not react with the polypeptide of interest. Such a kit may include a substantially isolated polypeptide antigen comprising an epitope which is specifically immunoreactive with at least one anti-polypeptide antigen antibody. Further, such a kit includes means for detecting the binding of said antibody to the antigen (e.g., the antibody may be conjugated to a fluorescent compound such as fluorescein or rhodamine which can be detected by flow cytometry). In embodiments, the kit may include a recombinantly produced or chemically synthesized polypeptide antigen. The polypeptide antigen of the kit may also be attached to a solid support.

In a further embodiment, the detecting means of the above-described kit includes a solid support to which said polypeptide antigen is attached. Such a kit may also include a non-attached reporter-labeled anti-human antibody. In this embodiment, binding of the antibody to the polypeptide antigen can be detected by binding of the said reporter-labeled antibody.

In an additional embodiment, the invention includes a diagnostic kit for use in screening serum containing antigens of the polypeptide of the invention. The diagnostic kit includes a substantially isolated antibody specifically immunoreactive with polypeptide or polynucleotide antigens, and means for detecting the binding of the polynucleotide or polypeptide antigen to the antibody. In one embodiment, the antibody is attached to a solid support. In an embodiment, the antibody is a monoclonal antibody. The detecting means of the kit may include a second, labeled monoclonal antibody. Alternatively, or in addition, the detecting means may include a labeled, competing antigen.

In one diagnostic configuration, test serum is reacted with a solid phase reagent having a surface-bound antigen obtained by the methods of the present invention. After binding with specific antigen antibody to the reagent and removing unbound serum components by washing, the reagent is reacted with reporter-labeled anti-human antibody to bind reporter to the reagent in proportion to the amount of bound anti-antigen antibody on the solid support. The reagent is again washed to remove unbound labeled antibody, and the amount of reporter associated with the reagent is determined. Typically, the reporter is an enzyme which is detected by incubating the solid phase in the presence of a suitable fluorometric, luminescent or colorimetric substrate (Sigma, St. Louis, Mo.).

The solid surface reagent in the above assay is prepared by known techniques for attaching protein material to solid support material, such as polymeric beads, dip sticks, 96-well plates and/or filter material. These attachment methods generally include non-specific adsorption of the protein to the support or covalent attachment of the protein, typically through a free amine group, to a chemically reactive group on the solid support, such as an activated carboxyl, hydroxyl, or aldehyde group. Alternatively, streptavidin coated plates can be used in conjunction with biotinylated antigen(s).

All publications, patents and patent applications cited herein, whether supra or infra, are hereby incorporated by reference in their entirety.

As used in this specification and the appended claims, the singular forms “a,” “an,” and “the” include plural referents unless the content clearly dictates otherwise. Thus, for example, reference to “an antigen” includes a mixture of two or more antigens, a reference to “a subject polypeptide” includes a plurality of such polypeptides, and reference to “the agent” includes reference to one or more agents and equivalents thereof known to those skilled in the art, and so forth.

It will be clear that the invention may be practiced otherwise than as particularly described in the foregoing description and examples. Numerous modifications and variations of the present invention are possible in light of the above teachings and, therefore, are within the scope of the appended claims.

It is to be understood that both the foregoing general description and the following detailed description are exemplary and explanatory only and are not restrictive of the invention, as claimed. Moreover, it must be understood that the invention is not limited to the particular embodiments described, as such may, of course, vary. Further, the terminology used to describe particular embodiments is not intended to be limiting, since the scope of the present invention will be limited only by its claim.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test the invention.

Further, all numbers expressing quantities of ingredients, reaction conditions, % purity, polypeptide and polynucleotide lengths, and so forth, used in the specification and claims, are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents to the scope of the claims, each numerical parameter should at least be construed in light of the number of reported significant digits, applying ordinary rounding techniques.

Nonetheless, the numerical values set forth in the specific examples are reported as precisely as possible. Any numerical value, however, inherently contains certain errors from the standard deviation of its experimental measurement.

All publications cited are incorporated by reference herein in their entireties; references cited in such publications are also incorporated by reference in their entireties.

### MODES OF PRACTICING THE INVENTION

### Embodiments of the Invention

The present invention provides newly identified APO2L and IL-24 variant polypeptides and nucleic acid molecules, as well as isolated polynucleotides encoding the polypeptides, and expression vectors containing the isolated polynucleotides.

Nucleic acid molecules of the invention include isolated cDNA, genomic DNA, cRNA, siRNA, RNAi, and mRNA molecules. They may be double-stranded, and may comprise sequences found in the Sequence Listing and/or complements of sequences found in the Sequence Listing, as well as biologically active fragments of any of these.

Isolated nucleic acid molecules of the invention also include those with a polynucleotide sequence at least 95% homologous to those described above. They further include those with a polynucleotide sequence that hybridizes under high stringency conditions to those described above. Yet further, they include complements of those sequences which hybridize under high stringency conditions. These complements include, but are not limited to, RNAi, anti-sense molecules, and ribozymes.

The invention also provides isolated polypeptides with an amino acid sequence found in the Sequence Listing, or a biologically active fragment of such an amino acid sequence. This polypeptide can be encoded by one or more nucleic acid molecule with a nucleotide sequence found in the Sequence Listing or a biologically active fragment of a nucleotide sequence found in the Sequence Listing.

The invention further provides a vector with one or more promoter that regulates the expression of an isolated nucleic acid molecule with a polynucleotide sequence, or a fragment of a polynucleotide sequence, found in the Sequence Listing. The promoter may or may not be naturally contiguous to the nucleic acid molecule; it may be inducible, conditionally active, e.g., the cre-lox promoter, constitutive, and/or tissue-specific.

The invention yet further provides a prokaryotic or eukaryotic recombinant host cell with an isolated nucleic acid molecule with a nucleotide sequence found in the Sequence Listing, and/or a biologically active fragment of such a sequence. This host cell may also include an isolated polypeptide with an amino acid sequence found in the Sequence Listing, and/or a biologically active fragment of such a sequence. This host cell may further include a vector, with the nucleic acid molecule described above and a promoter that regulates its expression. Eukaryotic host cells of the invention may be human, non-human mammalian, insect, fish, plant, or fungal cells.

The invention provides a non-human animal injected with or transformed with an isolated nucleic acid molecule with a nucleotide sequence found in the Sequence Listing, and/or a biologically active fragment of such a sequence.

The invention also provides nucleic acid compositions, polypeptide compositions, vector compositions, and host cell compositions of the nucleic acid molecules, polypeptides, vectors, and host cells described above. These compositions include a carrier, which may be, e.g., a pharmaceutically acceptable carrier, an excipient, and/or a buffer.

Properties of the disclosed APO2L and IL-24 variants include the ability to induce apoptosis of certain types of target cells. Among the types of cells that are killed by contact with the molecules of the invention are, for example, cancer cells such as leukemia, lymphoma, and melanoma or other tumor cells, and virally-infected cells.

In another aspect, the invention provides a method for producing the disclosed polypeptides. For example, cell free expression and culturing host cells transformed with a recombinant expression vector with nucleic acids encoding these polypeptides under appropriate expression conditions are included among the methods of the invention. Specifically, the invention provides a method of producing a recombinant host cell by providing a vector that includes an isolated nucleic acid molecule with a nucleotide sequence found in the Sequence Listing, and/or a biologically active fragment of such a sequence, and allowing the vector to contact a host cell, thus forming a recombinant host cell.

The invention also provides a method of producing a polypeptide by providing a recombinant host cell, such as described above, and culturing it to produce a polypeptide of the invention. The invention further provides a method of producing a polypeptide by providing an isolated nucleic acid molecule with a nucleotide sequence found in the Sequence Listing and/or a biologically active fragment of such a sequence, and expressing the nucleic acid molecule in a cell-free system to produce the polypeptide. This method can be performed using a wheat germ lysate, rabbit reticulocyte, or E. coli lysate expression system

In another aspect, the invention provides methods and compositions to modulate the polypeptides of the invention, and to prevent, and diagnose diseases associated with the polypeptides of the invention, and the polynucleotides that encode them. Specifically, the invention provides a diagnostic kit. The kit may include a vehicle and a polynucleotide molecule complementary to one with a nucleotide sequence found in the Sequence Listing and/or a biologically active fragment of such a sequence. The kit may also include a vehicle and an antibody that specifically binds to an isolated polypeptide with an amino acid sequence found in the Sequence Listing and/or a biologically active fragment of such a sequence. The kit may further include an isolated polypeptide with an amino acid sequence found in the Sequence Listing and/or a biologically active fragment of such a sequence.

The invention provides methods for determining the presence of a nucleic acid molecule with a nucleotide sequence found in the Sequence Listing or its complement, and/or a biologically active fragment of such a sequence. This method can be performed by providing a nucleic acid molecule with a nucleotide sequence found in the Sequence Listing or its complement, and/or a biologically active fragment of such a sequence, allowing the molecules to interact, and determining whether interaction has occurred.

The invention also provides methods for determining the presence in a sample of an antibody specific to a polypeptide of the invention, or a biologically active fragment, as described above, by providing a composition with the polypeptide, allowing the polypeptide to interact with the sample, and determining whether interaction has occurred.

The invention further provides antibodies that specifically bind to and/or interfere with the activity of one or more polypeptide, or a biologically active fragment of a polypeptide of the invention. The polypeptides bound to or inhibited by these antibodies are at least six contiguous amino acid residues in length, and are found in the Sequence Listing. Antibodies of the invention may be polyclonal; monoclonal; single chain; or be completely or partially comprised of active fragments, such as antigen-binding fragments, F, fragments, cdr fragments, and framework fragments.

In yet another aspect, the invention provides a method of inhibiting tumor growth by providing a composition that includes one or more isolated polypeptide or biologically active fragment thereof chosen from the Sequence Listing, and contacting the tumor with the composition. The invention provides a method for killing tumor cells including, but not limited to tumor cells that possess a death domain receptor by providing a composition including one or more polypeptide or biologically active fragment thereof chose from the Sequence Listing, and contacting the composition with the tumor cell. This method can kill human tumor cells, such as leukemic tumor cells or, e.g., carcinomas such as mammary adenocarcinomas non-small cell lung carcinomas, and tumor cells from tumors of the breast, colon, lung, prostate, bladder, stomach or skin.

The invention also provides specific types of tumors in subjects in need of such treatment by providing a composition that includes one or more polypeptides or biologically active fragments thereof chosen from the Sequence Listing, and contacting the composition with the tumor. Mammary adenocarcinomas, non-small cell lung carcinomas, breast tumors, lung tumors, prostate tumors, colon tumors, stomach tumors, bladder tumors, and skin cancers can be treated by this method.

The invention further provides for using a polypeptide or biologically active fragments thereof chose from the Sequence Listing as a target in screening for a modulator. Suitable modulator targets include small molecule drugs and antibodies.

The invention provides a method of stimulating an immune cell response, e.g., immune cell proliferation, by providing a composition that includes a substantially pure polypeptide or biologically active fragment thereof chosen from the Sequence Listing and either contacting the polypeptide with one or more immune cell or administering the composition to a subject. Immune cells which can be stimulated in this manner include monocytes, lymphocytes, macrophages, and peripheral blood mononuclear cells (PBMC). The substantially pure polypeptide or fragment thereof may be encoded by a nucleic acid molecule comprising a nucleotide sequence or biologically active fragment thereof chosen from the Sequence Listing. In embodiments that involve administering the composition to a subject, it may be administered either locally or systemically. Immune cells that can mediate the response include monocytes, lymphocytes, macrophages and PBMCs. The polypeptides of this method can be encoded by nucleic acid molecules or their biologically active fragments with nucleotide sequences chosen from the Sequence Listing.

The invention also provides a method of increasing the number of immune cells in a subject following cancer therapy, by providing a composition with a substantially pure polypeptide and/or one or more of its biologically active fragments chosen from the Sequence Listing, and administering the composition to the subject. This method can be used to increase the number of monocytes, lymphocytes, macrophages, or PBMCs. This method is suitable for use following chemotherapy radiation therapy and/or bone marrow transplantation for cancer. The polypeptides used in practicing this method can be encoded by nucleic acid molecules or their biologically active fragments with nucleotide sequences chosen from the Sequence Listing.

The invention further provides a method for treating or preventing an infection in a subject by providing a composition with a substantially pure polypeptide and/or one or more of its biologically active fragments chosen from the Sequence Listing, and administering the composition, either locally or systemically, to the subject. Bacterial, mycoplasma, fungal, and viral infections can be treated by this method. The polypeptide can be encoded by nucleic acid molecules or their biologically active fragments with nucleotide sequences chosen from the Sequence Listing.

The invention yet further provides a method of modulating an immune response in a subject by providing a modulator of a polypeptide and/or one or more of its biologically active fragments chosen from the Sequence Listing, and administering the modulator to a subject. The modulator may be an agonist and/or antagonist. Suitable modulators include antibodies, e.g., polyclonal antibodies, monoclonal antibodies, or cdr fragments, framework fragments, single chain antibodies, and active antibody fragments. The method can modulate a variety of immune responses, e.g., it can suppress inflammation and suppress autoimmune decrease. It can treat rheumatoid arthritis, osteoarthritis, psoriasis, inflammatory bowel disease, multiple sclerosis, and ischemia-related disorders, such as fulminant liver failure, myocardial infarction and stroke.

The invention provides a method of enhancing the immune response to a vaccine in a subject by providing a polypeptide and/or one or more of its biologically active fragments chosen from the Sequence Listing, providing a vaccine composition, and administering the polypeptide composition and the vaccine composition to the subject. The polypeptide composition can be administered to the subject either prior to, after, or substantially contemporaneously with the vaccine composition.

In another aspect, the present invention provides nucleic acid and polypeptide constructs for producing proteins in higher yields than when they are produced in their natural environment, and provides vectors, host cells, and methods for producing proteins in higher yields. The polypeptide or polynucleotide constructs can be modified, such as by the forming a fusion molecule with a fusion partner; these fusion molecules may be prepared by any conventional technique.

### EXAMPLES

### Example 1

### Cells and Cell Culture

Human kidney epithelial 293T cells (ATCC, cat# CRL-11268), human kidney epithelial 293 cells (ATCC, cat#CRL-1573), the human colon cancer cell line, COLO-205 cells (ATCC, cat# CCL-222), the human cervix cancer cell line, HeLa-229 cells (ATCC, Manassas, Va. cat# CCL-2.1), and human hepatocytes (Cambrex, Cat#AC-2625A) were purchased from American Type Culture Collection (ATCC, Manassas, Va.) and Cambrex (East Rutherford, N.J.).

The cells were grown in the basal medium supplemented with 10% fetal bovine serum (FBS, ATCC Cat#30-2020) and 1:100 penicillin-streptomycin (Cellgro, Hermdon, Va., cat#30-002-CI). The basal medium was DMEM (ATCC, Cat#30-2002) for 293T and 293 cells, RPMI-1640 medium (ATCC, cat#30-2001) for COLO-205 cells, Minimum Essential Medium Eagle (MEM, Mediatech, Hermdon, Va., CAT#MT 10-010-CM) for HeLa-229 cells, and Hepatocyte Culture Medium (HCM, Cambrex, CC-3198) for human hepatocytes.

### Example 2

### Plasmid Construction

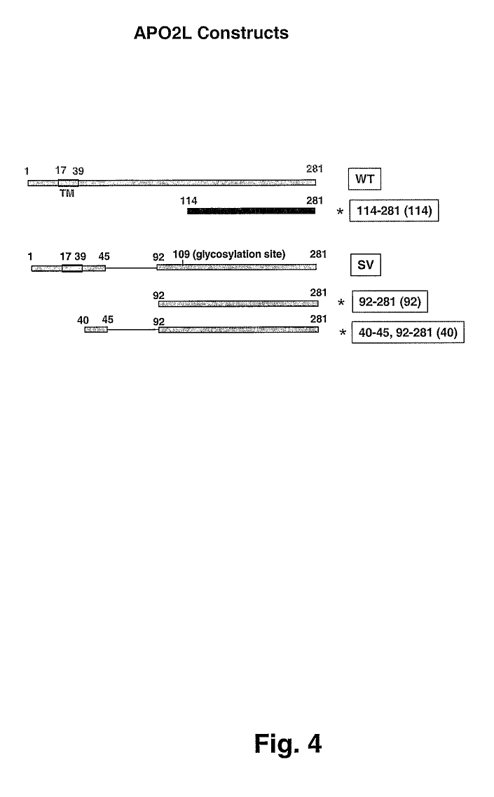

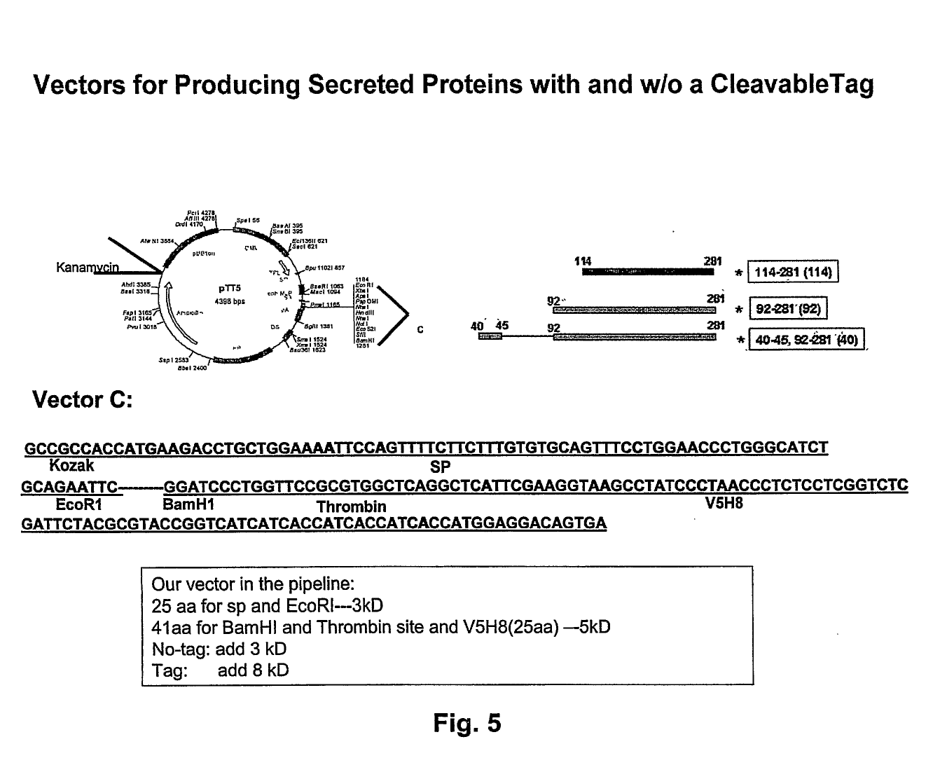

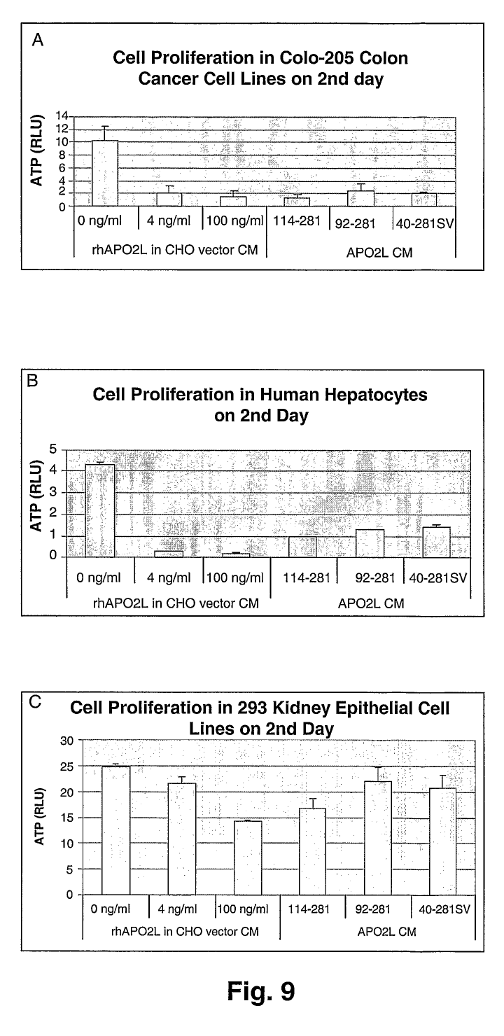

Fragments of the extracellular domain of APO2L corresponding to amino acid residues 40-45 and 92-281; 92-281; and 114-281, as shown in FIG. 4, were PCR-amplified utilizing EcoRI and BamHI subcloning sites and subcloned into a modified mammalian expression vector, as shown in FIG. 5 to produce Vector C, as shown in FIG. 9 Each fragment was PCR-amplified with or without a stop codon in order to produce protein without the V5H8 tag or with the V5H8 tag, respectively. The primers used for subcloning are as follows. For APO2L 40-45 plus 92-281 (i.e., a construct having amino acid residues 40-45 and 92-281 of the wild type APO2L or a polynucleotide sequence encoding such), without tag (APO2L 40-no tag), the forward primer was EcoRI_AACG AGCTGAAGCAGATGATTTTG and the reverse primer is BamHI_TTAGCCAACT AAAAAGGCCCCGA. For APO2L 40-45 plus 92-281 with tag (APO2L40-tag), the forward primer was EcoRI_AACGAGCTGAAGCAGATGATTTTG and the reverse primer was BamHI_GCCAACTAAAAAGGCCCCGAA. For APO2L 92-281 (i.e., a construct having amino acid residues 92-281 of the wild type APO2L or polynucleotide sequence encoding such) without tag (“APO2L 92-no tag”), the forward primer was EcoRI_ATTTTGAGAACCTCTGA GGAAAC and the reverse primer was BamHI_TTAGCCAACTAAAAAGGCCCCGA. For APO2L 92-281 with tag (“APO2L92-tag”), the forward primer was EcoRI_ATTTTGAGAACCTCT GAGGAAAC and the reverse primer was BamHI_GCCAACTAAAAA GGCCCCGAA. For APO2L 114-281 (i.e., construct having amino acid residues 114-281 of the wild type APO2L or polynucleotide sequence encoding such) without tag (“APO2L 114-no tag”), the forward primer was EcoRI_GTGAGAGAAAGAGGTCCTCAGA and the reverse primer was BamHI_TTAGCCAACTAAAAAGGCCCCGA. For APO2L 114-281 with tag (“APO2L 114-tag”), the forward primer was EcoRI_GTGAGAGAAAG AGGTCCTCAGA and the reverse primer was BamHI_GCCAACTAAAAA GGCCCCGAA.

### Example 3

### Gene Expression by Transfection

Human kidney epithelial 293T cells were plated at 5×10⁵ cells/well in 6-well plates or at 1.5×10⁴ cells/well in 96-well plates in a 2 ml volume of media in a 6-well plate or 100 μl media in a 96-well plate. The culture medium contained DMEM supplemented with 10% FBS and 1:100 penicillin-streptomycin. The cells were incubated at 37° C. with 5% CO₂ overnight.

All six APO2L constructs described in Example 2 were transfected to 293T cells using Fugene 6 Transfection Reagent (Roche Diagnostics, Indianapolis, Ind., Cat#1814443). Briefly, the medium was changed to 2 ml fresh culture medium per 6-well plate or 100 μl per 96-well plate at least 2 hours before transfection. Fugene 6, 5 μI, was added dropwise to 100 μl serum-free DMEM, mixed by tapping the microcentrifuge tube, and incubated at room temperature for five min. DNA (1.3 μg) was added dropwise to the Fugene 6/medium mixture and mixed, then incubated at room temperature for 15 min. Then the Fugene 6/medium/DNA mixture was gently added to the cells in a 6-well plate. The transfected cells were incubated at 37° C. with 5% CO₂ for 48 hours and the medium changed to 1 ml of CHO liquid Soy medium (Hyclone, Cat#SH30359). Forty-eight hours later, the supernatant (conditioned medium) containing one or more APO2L soluble proteins was collected after centrifuging for 10 min. at room temperature.

### Example 4

### Apoptosis Assay

Cells of a human colon cancer cell line, COLO 205 cells, and cells of a human cervical cancer cell line, HeLa 229 cells, were seeded at about 1×10⁴ cells/well in a 96-well white, clear bottom plate (Becton Dickinson Discovery Labware, Bedford, Mass. cat#353947) in 100 μl per well of culture medium and incubated at 37° C. with 5% CO₂ overnight.

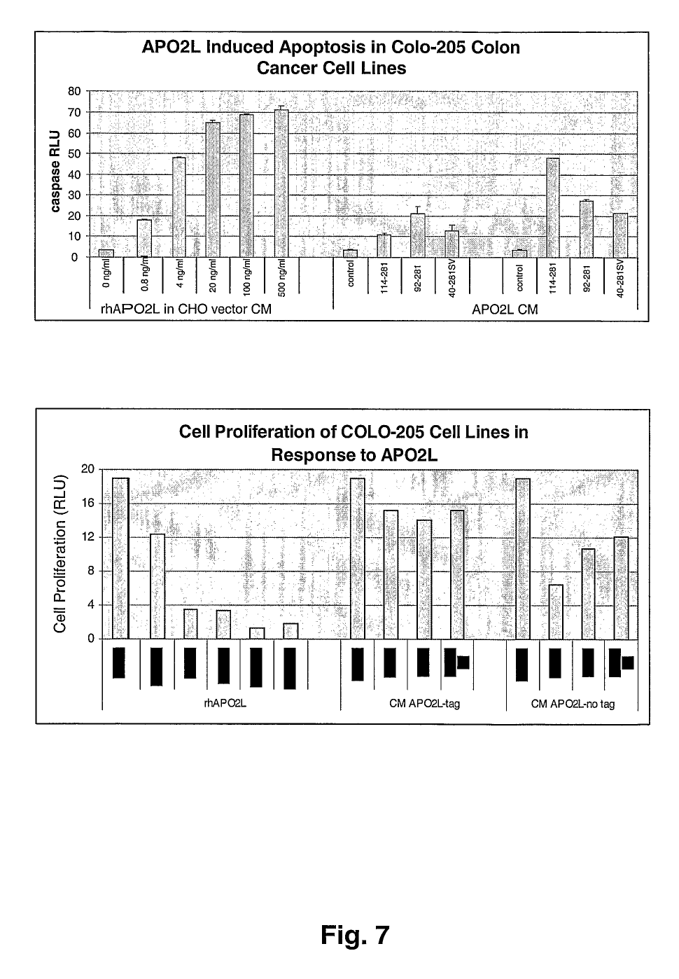

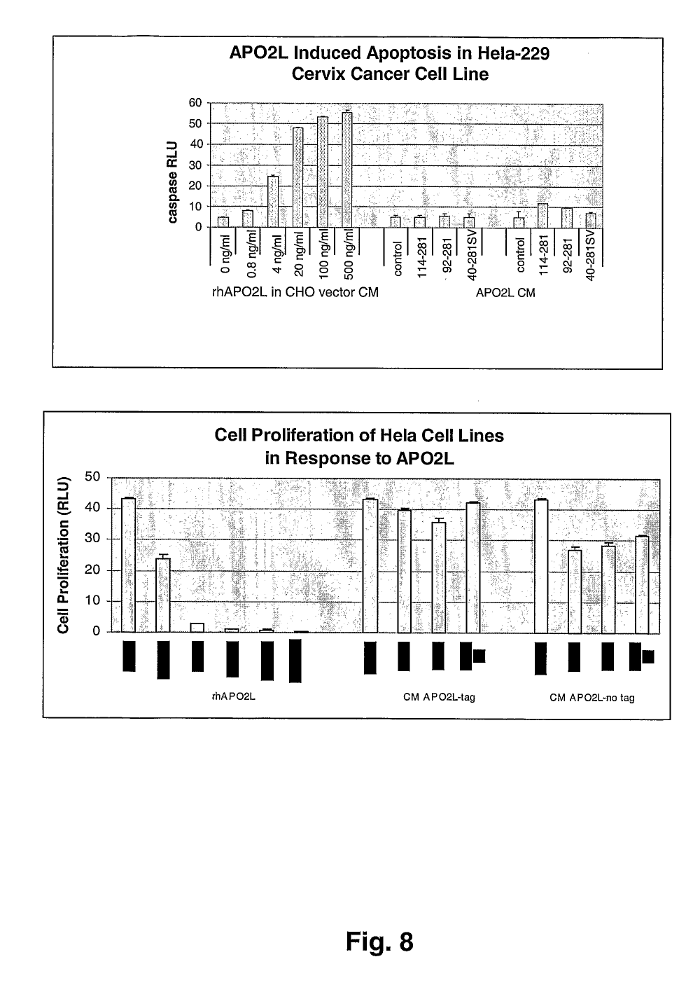

After aspirating the medium, the cells were treated with 50 μl or 100 μl of media conditioned with various APO2L constructs, including vector-only conditioned medium (Control), wild-type conditioned medium (114-281), and splice variant conditioned medium (92-281 and 40-281); recombinant human APO2L (rhAPO2L) was used as a positive control. Meanwhile, recombinant human APO2L (having an amino acid sequence identical to the 114-281 species of APO2L) (R&D, Minneapolis, Minn., cats 375-TEC) was serially diluted in vector-only conditioned medium, and applied to the cells; this served as a negative control. The cells were incubated at 37° C. with 5% CO₂. Approximately 3-6 hours later, a volume of apoptosis reagent equal to the volume of them was added. This Caspase-Glo 3/7 Reagent (Promega, Madison, Wis., Cat.# G8091) was incubated at room temperature for 30 min. to 3 hours in the dark. The luminescent signal was read using Lmax microplate reader (Molecular Devices, Sunnyvale, Calif.) with an integration time of 0.1-1.0 second. The results are shown in FIGS. 7 and 8.

Results shown in FIG. 7 demonstrate a dose reponse curve with regard to use of recombinant human APO2L (rhAPOL2L) in induction of caspase 3/7 in COLO-205 cells. Conditioned media from 293T cells transfected with the present constructs made with V5H8 tags induced caspase production to a certain extent, such as to the level of 0.8 ng/ml range as compared to the rhAPO2L. In contrast, conditioned media from constructs made in the absence of the V5H8 tags, especially the 114-281 construct (i.e., 293T cells transfected with vector containing DNA encoding amino acid residues 114-281 of the wild type APO2L sequence), were more efficient in inducing caspase production in the COLO-205 cells than the cells transfected with constructs with tags, such as up to the level induced by 4 ng/ml of rhAPO2L or greater than the level induced by 9.8 ng/ml of rhAPO2L. These experiments were conducted using 50 μl of conditioned media.

Results shown in FIG. 8 demonstrate that the conditioned media from the 293T cells transfected with the present constructs were less efficient in inducing caspase production in Hela-229 cells than in the COLO-205 cells for all the constructs tested, with the conditioned media from the 293T cells transfected with the untagged constructs inducing a level of caspase production in the range of that induced by 0.8 ng/ml of rhAPO2L.

### Example 5

### Cell Proliferation Assay for COLO-205 Cells and Normal Cells

COLO205 cells, HeLa229 cells, 293 cells, and human hepatocytes were seeded at about 1×10⁴ cells/well in 96-well white with clear bottom plates (Becton Dickinson Discovery Labware, Bedford, Mass. cat#353947) in 100 μl per well of culture medium (RMPI-1640 supplemented with 10% fetal bovin serum and antibiotics) and incubated at 37° C. with 5% CO₂ overnight.

After aspiration of the medium, fresh culture medium was added to the cells. The cells were treated with 50 ul of different APO2L conditioned medium in a 1:1 dilution, including vector-only conditioned medium. Meanwhile, recombinant APO2L (R&D, Minneapolis, Minn., cat# 375-TEC) was serially diluted in vector-only conditioned medium, and applied to the cells; this served as the negative control. The cells were incubated at 37° C. with 5% CO₂. After various incubation times (several hours up to 4 days), an equal volume of CellTiter-Glo Buffer (Promega, Madison, Wis., Cat# G7570) was added to the culture medium to determine the number of viable cells, based on detection of ATP in viable cells, according to the manufacturer's procedure. Briefly, cells were lysed in CellTiter-Glo Reagent and plated at room temperature for 10 min. to stabilize the luminescence signal, which was then read luminescence on a Lmax microplate reader (Molecular Devices, Sunnyvale, Calif.) with an integration time of 0.1 second. Results are shown in FIGS. 7, 8, and 9.

FIG. 7 shows that rhAPO2L inhibits COLO-205 cell proliferation at the 0.8 ng/ml concentration and significantly inhibits COLO-205 cell proliferation at the 4 ng/ml concentration. Conditioned media from cells transfected with tagged and untagged constructs showed varying levels of inhibition of cell proliferation. Significant inhibition of proliferation in the COLO-205 cells was observed in the conditioned media from cells transfected with untagged APO2L constructs, equal to or greater than the effect of 0.8 ng/ml or rhAPO2L. Inhibition of cell proliferation as measured herein may reflect cell survival as a combination effect of cell killing and inhibition of proliferation.

FIG. 9 shows the results of treatment of COLO-205 colon cancer cells, human hepatocytes, and 293 kidney epithelial cells, respectively, with 40 μl of conditioned media from 293 cells transfected with the different APO2L constructs, 114-281, 92-281 or 40-45,92-281 (the “40-281” construct) described above, as compared to treatment with rhAPO2L at the 0, 4, or 100 ng/ml concentration, on cell proliferation after a 24 hr treatment. FIG. 9 also shows inhibition of COLO-205 cells proliferation by all the conditioned media tested, substantially to the same extent as that produced by 4 or 100 ng/ml of rhAPO2L. FIG. 9 further shows inhibition of proliferation of human hepatocytes, demonstrating that the 40-45,92-281 species is less inhibitory to the proliferation of normal human hepatocytes than the 92-281 species, which is less inhibitory than the 114-281 species of APO2L fragments. FIG. 9 shows that the 92-281 species is less inhibitory on proliferation of 293 kidney epithelial cells than the 40-42,92-281 species, which is less inhibitory than the 114-281 species.

### Example 6

### Apoptosis of Activated T-Cells

Activated human T cells are induced to undergo programmed cell death (apoptosis) upon its triggering through the CD3/T cell receptor complex, a process termed activated-induced cell death (AICD). AICD of CD4T cells isolated from HIV-infected asymptomatic individuals has been reported (Groux et al., supra). Thus, AICD may play a role in the depletion of CD4⁺T cells and the progression to AIDS in HIV-infected individuals. Accordingly, APO2L polynucleotides, polypeptides, or their antagonists are administered to HIV infected cells, in vivo, or in vitro or ex vivo, thus inhibiting APO2L or IL-24-mediated T cell death. Patients may be symptomatic or asymptomatic when treatment with APO2L polynucleotides, polypeptides, or their antagonists commences. Prior to treatment, peripheral blood T cells may be extracted from the patient and tested for susceptibility to APO2L-mediated cell death by procedures known in the art.

A patient's blood or plasma is contacted with APO2L antagonists (e.g., anti-APO2L antibodies) of the invention ex vivo. The APO2L antagonists may be bound to a suitable chromatography matrix by procedures known in the art. The patient's blood or plasma flows through the chromatography matrix, e.g., a column, containing APO2L antagonist bound to the matrix, before being returned to the patient. The immobilized APO2L antagonist binds APO2L, thus removing APO2L protein from the patient's blood.

The APO2L polynucleotide, polypeptide, or antagonist may be administered alone or in combination with other inhibitors of T cell apoptosis. For example, as discussed above, Fas-mediated apoptosis has been implicated in loss of T cells in HIV positive individuals (Katsikis et al., J Exp. Med., 181:2029-2036 (1995)). Thus, a patient susceptible to both Fas ligand mediated and APO2L-mediated T cell death may be treated with both an agent that blocks APO2L receptor interactions and an agent that blocks Fas-ligand/Fas interactions. Suitable agents for blocking the binding of Fas-ligand to Fas include, but are not limited to, soluble Fas polypeptides; multimeric forms of soluble Fas polypeptides (e.g., dimers of sFas/Fc); anti-Fas antibodies that bind Fas without transducing the biological signal that results in apoptosis; anti-Fas-ligand antibodies that block binding of Fas-ligand to Fas; and muteins of Fas-ligand that bind Fas but do not transduce the biological signal that results in apoptosis. The anti-Fas antibodies may be monoclonal antibodies. Examples of suitable agents for blocking Fas-ligand/Fas interactions, including blocking anti-Fas monoclonal antibodies, are described in WO 95/10540, hereby incorporated by reference.

### Example 7

### Blocking Agents Combined with Antagonists

Agents which block binding of APO2L to an APO2L receptor are administered with the APO2L polynucleotides, polypeptides, or antagonists of the invention. Such agents include, but are not limited to, soluble APO2L receptor polypeptides; multimeric forms of soluble APO2L receptor polypeptides; and APO2L receptor antibodies that bind the APO2L receptor without transducing the biological signal that results in apoptosis; anti-APO2L antibodies that block binding of APO2L to one or more APO2L receptors; and muteins of APO2L that bind APO2L receptors but do not transduce the biological signal that results in apoptosis.

### Example 8

### Use of a Cell-Free Expression System for Expression of Mature Secreted Proteins

A nucleotide primer is designed and synthesized that contains the following nineteen nucleotides 5′CCACCCACCACCACCAATG 3′ followed by the first nineteen nucleotides predicted to encode the amino terminus of a mature secreted protein. To express the mature secreted protein, a second reverse primer is designed to a region of the plasmid approximately 1000 nucleotides downstream from the coding sequence of the gene to be expressed. The second primer is designed as the reverse complement of the vector sequence in this region, such that this primer will be useful for PCR amplification of the mature coding sequence of the mature open reading frame to be expressed. The second primer is typically 17-23 nucleotides in length with a Tm of approximately 55-65° C.

A purified plasmid containing the cDNA to be expressed, or E. coli cells containing the plasmid that contains the cDNA to be expressed, is then added as a template to a standard PCR reaction that includes the two primers described above, standard PCR reagents, and a DNA polymerase that has proof-reading activity, and the plasmid is subjected to 15-30 cycles of PCR amplification. The product of this PCR reaction is the PCR1 coding template.

Optionally, a separate PCR reaction is setup to prepare a GST-Mega primer that will be used to create a GST-fusion expression template. Using a plasmid template that contains the coding sequence for GST downstream of the Non-Omega translation initiation sequence, a PCR reaction is prepared using the primer 5′ GGTGACACTATAGAACT CACCTATCTCCCCAACA 3′ and the primer 5′ GGGCCCCTG GAACAGAACTTC 3′ and amplified in a standard PCR reaction that includes the two primers described above, standard PCR reagents, and a DNA polymerase that has proof-reading activity; the reaction includes 15-30 cycles of PCR amplification. After the PCR reaction is complete the PCR product is subjected to exonuclease I treatment for 30 min. at 37° C., then heat-inactivated at 80° C. for 30 min., and the PCR product is purified by agarose gel electrophoresis and extracted using a gel purification kit (Amersham) to produce the GST-Mega primer.

The GST-Mega primer is then used to create GST-fusion expression templates containing the mature coding region of the cDNA to be expressed. An aliquot of the PCR1 coding template (0.5 ul) is mixed with an aliquot of the GST-Mega primer (1 ul) and a primer (5′ GCGTAGCATTTAGGTGACACT 3′) that encodes part of the SP6 promoter sequence and anneals to the five prime end of the GST Mega primer, and with a second primer designed to a region of the plasmid approximately 300-350 nucleotides downstream from the coding sequence of the gene to be expressed. This second primer is designed as the reverse complement of the vector sequence in this region such that this primer will be useful for doing PCR amplification of the PCR1 coding template. This second primer is typically 17-23 nucleotides in length with a Tm of approximately 55-65° C. The GST-fusion expression template is then generated a standard PCR reaction using standard PCR reagents, a DNA polymerase that has proof-reading activity, and 15-30 cycles of amplification. The product of this PCR reaction is called the GST-fusion expression template.

An in vitro transcription reaction (50 μl) is then prepared using 5 μl of the GST-fusion expression template in the following buffer, 80 mM HEPES KOH pH 7.8, 16 mM Mg(OAc)₂, 2 mM spermidine, 10 mM DTT containing 1 unit of SP6 (Promega) and 1 unit of RNasin (Promega) and incubated for 3 hours at 37° C. The mRNA is then subjected to ethanol precipitation by addition of 20 μl of RNase-free water, 37.5 μl of 5 M ammonium acetate, and 862 ul of 99% ethanol, mixed by vortexing, and pelleted by centrifugation at 15,000×g for 10 min. at 4° C. The mRNA pellet is then washed in 70% ethanol and again pelleted by centrifugation at 15,000×g for 5 min. at 4° C.

For the i71 vitro translation reaction, a stock of 2× Dialysis Buffer is prepared that contains 20 mM HEPES buffer pH 7.8 (KOH), 200 mM KOAc, 5.4 mM Mg(OAc)₂, 0.8 mM spermidine, 100 μM DTT, 2.4 mM ATP, 0.5 mM GTP, 32 mM creatine phosphate, 0.02% NaN₃, and 0.6 mM Amino Acid Mix minus ASP, TRP, GLU, ISO, LEU, PHE, and TYR. The amino acids ASP, TRP, GLU, ISO, LEU, PHE, and TYR are prepared separately as an 80 mM stock in 1N HCL, and, after complete dissolution, are added to a final concentration of 0.6 mM. After addition of all ingredients the 2× Dialysis Buffer stock is adjusted to pH 7.6 using 5 N KOH, filter sterilized, and stored frozen in aliquots at −80° C.

To resuspend the iii vitro transcribed mRNA that has been ethanol-precipitated and washed in 70% ethanol, a 50 ul translation mixtu-re is prepared that includes Wheat Germ Reagent at a final OD 260 nm of 60, and a volume of 1× Dialysis Buffer (to which 2 mM DTT has been added) sufficient to bring the final volume to 50 ul (Wheat Germ Reagent comprises 1× Dialysis Buffer). After removing the ethanol from the precipitated mRNA, the 50 ul translation mixture is added, allowed to sit for 5-10 min. and then the mRNA is resuspended. The complete translation mixture containing the resuspended mRNA is then layered under 250 ul of 1× Dialysis Buffer in a well of a 96-well round bottom microtiter plate, to setup the Bilayer Reaction. The plate is then sealed manually with a plate sealer and then incubated for 20 hours at 26° C.

To recover the recombinant protein expressed as a GST fusion, the translation mixture is transferred to a tube, diluted five-fold with phosphate buffered saline containing 0.25 M sucrose, 2 mM DTT, and 10 ul of glutathione-sepharose is added and incubated with mixing, for 3 hours at 4° C. The sepharose beads containing the bound GST fusion protein are then washed three times in phosphate buffered saline containing 0.25 M sucrose and 2 mM DTT. The beads are then washed with protease cleavage buffer containing 50 mM Tris pH 7.4, 150 mM NaCl, 1 mM EDNA, 2 mM DTT, and 0.25 M sucrose. After careful removal of the wash buffer 10 ul of final wash buffer, is added with 0.4 ul of Prescission Protease (Amersham), the beads gently suspended with a pipette, and then allowed to incubate overnight at 4° C. To recover the cleaved, secreted protein product, 20 ul of final wash buffer is added and entire liquid fraction recovered by pipette or by filtering through a sintered frit. To stabilize the recovered secreted protein, purified BSA prepared as a 10 mg/mI stock in PBS is added to a final concentration of 1 mg/ml and the protein sample then dialyzed in PBS and filter sterilized for storage prior to testing for biological activity. To produce additional protein the single Bilayer Reaction can be reproduced many times and the purification and formulation scaled accordingly. Typically, sixteen Bilayer Reactions will produce sufficient biologically active protein for testing in biological assays.

### Example 9

### Identifying Proteins Secreted at High Levels

Complementary DNAs (cDNAs) were analyzed bioinformatically to determine the nucleotide sequences that encode strong secretory leader sequences of secreted proteins, based upon a defined set of attributes that included, for example, the presence of hydrophobic signal peptide domains typically encoded by the first 6-27 amino acid codons (18-81 nucleotides) of the open reading frame (ORF), beginning with 1-4 polar amino acids followed by a stretch of hydrophobic amino acids and then a short region of charged amino acids just before the cleavage site and the presence or absence of a cleavage site. Using such criteria, in addition to other physical characteristics, the signal peptide or leader sequence of a secreted protein, was determined.

In addition to bioinformatic analyses, strong secretory leader sequences were identified experimentally as well. cDNAs predicted to encode secreted proteins were subcloned into a pTT5 expression vector in frame with a C-terminal V5 and His ×8 epitope and transiently transfected into 293T cells using a 96-well high throughput system. Purified plasmid DNA for each clone was prepared using the Qiagen™ Turbo DNA system in 96 well plates. The DNA concentration for each clone was determined by absorbance at 260 nm and diluted to 50 μg/ml. Transient transfection cf ten 96-well plates was performed by combining 10 μg of each DNA plasmid with 50 μg GIBCO Opti-MEM I (Cat#:319-85-070) in a round bottom 96-well polystyrene plate (master transfection plate). In order to generate the transfection complex, 37.5 μg of Opti-MEM I, preincubated for 5 min. with 2.5 μg Fugene 6 (Roche Applied Science cat#:1988387), was added, and the complex allowed to form at room temperature for about 30 min.

The transfection complex was subsequently diluted by the addition of 100 μl of Opti-MEM I, mixed several times by pipetting in an up and down motion, and then transferred, 20 μl at a time, into ten 96 well flat bottom poly-lysine-coated plates (Becton Dickinson cat#: 356461). 293T cells suspended (200 μl at 2×10⁵ cell/ml) in DMEM medium containing 10% FBS, penicillin, and streptomycin were then added to each well and incubated at 37° C. in 5% CO₂. After approximately 40 hours, the medium was removed by aspiration, the cells briefly washed with 150 μl phosphate-buffered saline (PBS), and new pre-warmed medium added. To measure the expression and secretion level of each protein, fresh HyQ-PF CHO Liquid Soy (Hyclone Cat# SH30359.02) medium (150 μl) was added to each well, and incubated at 37° C. in 5% CO)₂. For measuring activity of secreted proteins, fresh DMEM medium containing 5% FBS and penicillin and streptomycin (150 μl) was added in place of the HyQ-PF CHIO Liquid Soy.

After an additional 48 hours, the culture supernatants from all ten 96-well plates were harvested and combined into a single sterile deep well plate, covered with a sterile lid, and centrifuged at 1400 RPM for 10 min. to pellet any loose cells and cell debris. The supernatant was then transferred to a new sterile deep well plate for testing for protein expression by Western blot. The cell layer remaining on the plates was solubilized with 0.2% SDS, 0.5% NP-40 in PBS.

The expression of cDNAs in 293-6E cells was tested either using the high throughput transfection process, or in larger quantities, using 293-6E cells grown in shaker flasks. For the high throughput process, 293-6E cells were treated in an identical fashion as 293T cells. For scale-up expression, 293-6E cells were grown in polycarbonate Erlenmeyer flasks fitted with a vented screw cap and rotated on a table top shaker at 100 RPM in Freestyle Medium (Invitrogen®) at 37° C. in 5% CO₂ at cell densities ranging from 0.5 to 3×10⁶ cells/ml. Typically, 50 ml of culture was grown in a 250 ml flask. One day prior to setting up a transfection, 293-6E cells were diluted into fresh Freestyle medium to a density of 0.6×10⁶ cells/ml. On the day of transfection, the cells were predicted to be in log phase (0.8−1.5×10⁶ cells/ml ) and adjusted to a density of 106 cells/ml.

To prepare the transfection mix, 2.5 ml sterile PBS was added to two 15 ml tubes; 50 μg DNA was added to one tube; into the other, 100 μl PEI solution (1 mg/ml sterile stock solution with polyethylenimine, linear, 25 kDa., pH 7.0 (Polysciences, Warrington, Wis.) was added. The solutions were then combined and allowed to incubate for 15 min. at room temperature to form the transfection complex. The transfection mixture was then transferred to a 293-6E suspension culture and allowed to grow for 4-6 days at 37° C. in 5% CO₂.

To determine protein secretion levels, culture supernatants were analyzed by Western blot. Samples (15 μl) were resolved by SDS-PAGE on a 26 lane Criterion gel (BioRad), transferred to nitrocellulose, blocked, and then probed with an anti-V5 HRP conjugate (Invitrogen®). Secretion levels were determined by comparing band intensity to that of one of three different purified standards run in the same Western analysis at three different concentrations. The standards used were either (1) V5-Hisx6 tagged Delta-like protein 1 extracellular protein; (2) V5-Hisx6 tagged CSF-1 Receptor extracellular domain, each expressed separately using the baculovirus expression system- and purified to >90% purity; or (3) Positope (Invitrogen, cat#: R900-50) containing a V5 Hisx6 tag. The standards were either run separately or in combination.

From the analysis of the high throughput expression of many cDNAs in 293T′ cells, several cDNA were identified that resulted in very high secretion levels. One of these, the signal peptide (leader) sequence from the high expressing clone. CLN00517648, which encoded human collagen, type IX, alpha 1, long form, was used to engineer the high level secretion of lower-expressing cDNAs, type I TM proteins, and type II cDNAs by replacing the exdogenous signal peptide sequence of each cDNA with that of collagen type IX, alpha 1. Constructs encoding human APO2L were engineered in the pTT5 vector and transfected into 293T and 293-6E cells to test expression and secretion using the improved signal peptide in 293T cells and in 293-6E cells, using both the high throughput and the scale-up procedures.

### Example 10

### Construction of Vector C

Vector C was constructed as shown in FIG. 5 with the appropriate polynucleotide sequence of the secreted protein to be expressed inserted between the EcoR1 site and the BamH1 site. Vector C was inserted into pTT5 for use in transfecting cells, such as 293 cells for production of the secreted protein.

### Example 11

### High Throughput Screening of Secreted Factors

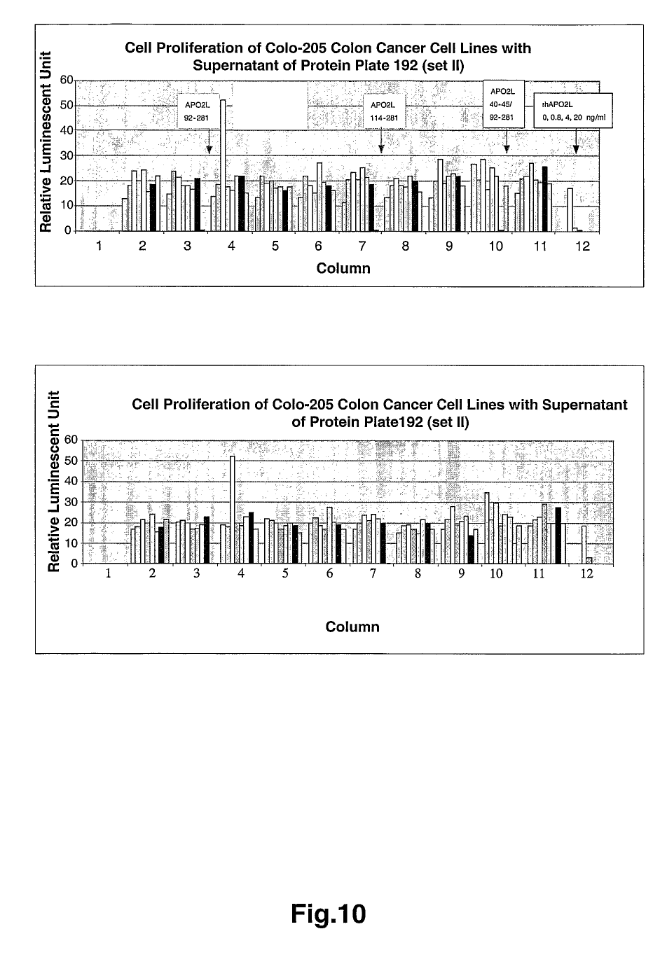

Conditioned media from 293 cells transfected with different mature secreted proteins, as described above, were used in a high throughput screening assay for cell proliferation and factors that inhibited cell proliferation was identified. Results are shown in FIG. 10. This histogram shows that APO2L 92-281, APO2L 114-281, APO2L 40-45/90-281 inhibited proliferation of COLO-205 cells to the same extent and approximately to the same extent as 4 or 20 ng/ml of rhAPO2L.

### Example 12

### Effect of APO2L Fragments Made in a Cell-Free Expression System on Proliferation of COLO-205

The colon cancer cell line Colo-205, purchased from American Type Culture Collection (ATCC, Manassas, Va., cat# CCL-222), was seeded at about 4×10⁴ cells/well in 96-well white with clear bottom plate (Becton Dickinson Discovery Labware, Bedford, Mass. cat#353947) in 200 μl per well of culture medium including RPMI-1640 medium (ATCC, cat# 30-2001) supplemented with 10% fetal bovine serum (FBS, ATCC Cat# 30-2020) and 1% of penicillin-streptomycin (Cellgro, Herndon, Va., cat#30-002-CI). The plate was sealed with Breathe Easy Sealing Tape (E&K scientific, Cambell, Calif., cat# T796200), and kept at room temperature for one hour followed by incubation at 37° C. and with 5% CO₂ overnight.

Recombinant APO2L (R&D, Minneapolis, Minn., cat# 375-TEC) was diluted in cell-free mock solution to 0 ng/ml, 0.8 ng/ml, 4 ng/ml, and 20 ng/ml, respectively. The sealing tape was peeled off. Cells were treated with the above serial concentration of recombinant APO2L with 1:1 dilution of COLO-205 culture medium to reach a total volume of 100 μl. The final concentrations of APO2L were 0 ng/ml, 0.4 ng/ml, 2 ng/ml, and 10 ng/ml, respectively. Cells were also treated with cell-free (CF) APO2L including CF_APO2L40, CF_APO2L92 and CF-APO2L114 with 1:1 dilution using COLO-205 culture medium with a final volume of 100 μl. The plate was sealed with Breathe Easy Sealing Tape. Cells were incubated at 37° C. and 5% CO₂ for four days.

CellTiter-Glo™ Luminescent Cell Viability Assay kit (Promega, Madison, Wis., Cat# G7570) was used to determine the number of viable cells by following the manufacture's procedure. Briefly, CellTiter-Glo Buffer was transferred into an amber bottle containing CellTiter-Glo Substrate to make CellTiter-Glo Reagent. About 100 μl (equal volume cell culture medium) of CellTiter-Glo Reagent was applied to each well. The contents were mixed for 2 minutes on a shaker to induce cell lysis. The plate was allowed to stand at room temperature for 10 minutes to stabilize the luminescence signal. Luminescence was read using Lmax microplate reader (Molecular Devices, Sunnyvale, Calif.) with an integration time of 0.1 second.

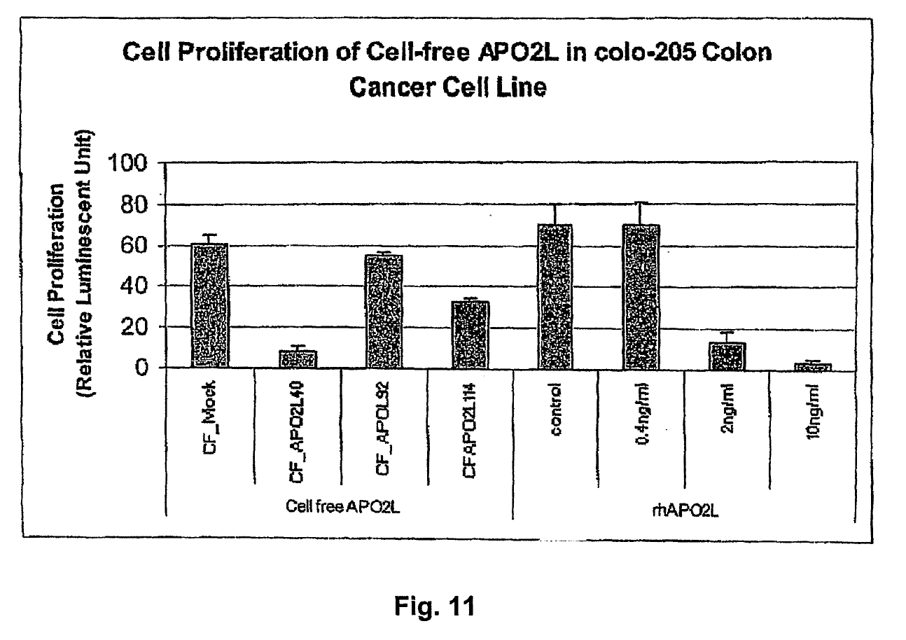

The APO2L fragments, the 114, 92, and 40 constructs, were made in a cell-free expression system as described in Example 8 above. Results are shown in FIG. 11. FIG. 11 shows that the APO2L40 construct caused significantly more inhibition of proliferation than either the APO2L92 construct or the APOL2L114 construct, with the APO2L40 construct having an activity comparable to that of rhAPO2L at between 2-10 ng/ml.

Tables

TABLE 1
SEQ ID NOS.: 1-223

| | SEQ. ID. | SEQ. ID. | SEQ. ID. | | |
|---|---|---|---|---|---|
| FP ID | NO.: (N1) | NO.: (P1) | NO.: (N0) | Source ID | Type |
| HG1015090 | SEQ. ID. | SEQ. ID. | SEQ. ID. | CLN00493987_5pv1.a | IL24 |
| | NO.: 1 | NO.: 6 | NO.: 11 | | |
| HG1015091 | SEQ. ID. | SEQ. ID. | SEQ. ID. | NP_006841:NM_006850 | IL24 |
| | NO.: 2 | NO.: 7 | NO.: 12 | | |
| HG1015092 | SEQ. ID. | SEQ. ID. | SEQ. ID. | CLN00453866_5pv1.a | IL24 |
| | NO.: 3 | NO.: 8 | NO.: 13 | | |
| HG1015093 | SEQ. ID. | SEQ. ID. | | NP_006841:NM_006850_exon1 | IL24 |
| | NO.: 4 | NO.: 9 | | | |
| HG1015094 | SEQ. ID. | SEQ. ID. | | NP_006841:NM_006850_exon4 | IL24 |
| | NO.: 5 | NO.: 10 | | | |
| HG1014901 | SEQ. ID. | SEQ. ID. | SEQ. ID. | CLN00108891_5pv1.a | APO2 |
| | NO.: 14 | NO.: 15 | NO.: 16 | | |
| HG1019036 | SEQ. ID. | SEQ. ID. | | CLN00108891_frag1 | APO2 |
| | NO.: 17 | NO.: 21 | | | |
| HG1019037 | SEQ. ID. | SEQ. ID. | | CLN00108891_frag2 | APO2 |
| | NO.: 18 | NO.: 22 | | | |
| HG1019038 | SEQ. ID. | SEQ. ID. | SEQ. ID. | NP_003801:NM_003810 | APO2 |
| | NO.: 19 | NO.: 23 | NO.: 25 | | |
| HG1019040 | SEQ. ID. | SEQ. ID. | | NP_003801:NM_003810_frag1 | APO2 |
| | NO.: 20 | NO.: 24 | | | |
| HG1018265 | | SEQ. ID. | | collagen_leader_seq | leader |
| | | NO.: 26 | | | sequence |
| HG1018268 | | SEQ. ID. | | 112907:21594845_1-17 | HMM_SP |
| | | NO.: 27 | | | leader |
| | | | | | sequence |
| HG1018269 | | SEQ. ID. | | 112907:21594845_1-13 | leader |
| | | NO.: 28 | | | sequence |
| HG1018270 | | SEQ. ID. | | 112907:21594845_1-19 | leader |
| | | NO.: 29 | | | sequence |
| HG1018271 | | SEQ. ID. | | 112907:21594845_1-16 | leader |
| | | NO.: 30 | | | sequence |
| HG1018272 | | SEQ. ID. | | 112907:21594845_1-15 | leader |
| | | NO.: 31 | | | sequence |
| HG1018274 | | SEQ. ID. | | 13325208:13325207_1-30 | HMM_SP |
| | | NO.: 32 | | | leader |
| | | | | | sequence |
| HG1018275 | | SEQ. ID. | | 13325208:13325207_1-25 | leader |
| | | NO.: 33 | | | sequence |
| HG1018276 | | SEQ. ID. | | 13325208:13325207_1-33 | leader |
| | | NO.: 34 | | | sequence |
| HG1018277 | | SEQ. ID. | | 13325208:13325207_1-24 | leader |
| | | NO.: 35 | | | sequence |
| HG1018278 | | SEQ. ID. | | 13325208:13325207_1-26 | leader |
| | | NO.: 36 | | | sequence |
| HG1018279 | | SEQ. ID. | | 13325208:13325207_1-32 | leader |
| | | NO.: 37 | | | sequence |
| HG1018280 | | SEQ. ID. | | 13325208:13325207_1-27 | leader |
| | | NO.: 38 | | | sequence |
| HG1018281 | | SEQ. ID. | | 13325208:13325207_1-23 | leader |
| | | NO.: 39 | | | sequence |
| HG1018282 | | SEQ. ID. | | 13325208:13325207_1-35 | leader |
| | | NO.: 40 | | | sequence |
| HG1018284 | | SEQ. ID. | | 13938307:13938306_1-24 | HMM_SP |
| | | NO.: 41 | | | leader |
| | | | | | sequence |
| HG1018285 | | SEQ. ID. | | 13938307:13938306_1-21 | leader |
| | | NO.: 42 | | | sequence |
| HG1018287 | | SEQ. ID. | | 14718453:14718452_1-19 | HMM_SP |
| | | NO.: 43 | | | leader |
| | | | | | sequence |
| HG1018288 | | SEQ. ID. | | 14718453:14718452_1-15 | leader |
| | | NO.: 44 | | | sequence |
| HG1018289 | | SEQ. ID. | | 14718453:14718452_1-17 | leader |
| | | NO.: 45 | | | sequence |
| HG1018291 | | SEQ. ID. | | 15929966:15929965_1-23 | HMM_SP |
| | | NO.: 46 | | | leader |
| | | | | | sequence |
| HG1018293 | | SEQ. ID. | | 16356651:16356650_1-21 | leader |
| | | NO.: 47 | | | sequence |
| HG1018294 | | SEQ. ID. | | 16356651:16356650_1-17 | leader |
| | | NO.: 48 | | | sequence |
| HG1018296 | | SEQ. ID. | | 18204192:18204191_1-19 | HMM_SP |
| | | NO.: 49 | | | leader |
| | | | | | sequence |
| HG1018297 | | SEQ. ID. | | 18204192:18204191_1-22 | leader |
| | | NO.: 50 | | | sequence |
| HG1018298 | | SEQ. ID. | | 18204192:18204191_1-18 | leader |
| | | NO.: 51 | | | sequence |
| HG1018299 | | SEQ. ID. | | 18204192:18204191_1-16 | leader |
| | | NO.: 52 | | | sequence |
| HG1018300 | | SEQ. ID. | | 18204192:18204191_1-14 | leader |
| | | NO.: 53 | | | sequence |
| HG1018302 | | SEQ. ID. | | 23503038:15778555_1-20 | leader |
| | | NO.: 54 | | | sequence |
| HG1018303 | | SEQ. ID. | | 23503038:15778555_1-16 | leader |
| | | NO.: 55 | | | sequence |
| HG1018304 | | SEQ. ID. | | 23503038:15778555_1-21 | leader |
| | | NO.: 56 | | | sequence |
| HG1018306 | | SEQ. ID. | | 27479535:27479534_1-24 | HMM_SP |
| | | NO.: 57 | | | leader |
| | | | | | sequence |
| HG1018307 | | SEQ. ID. | | 27479535:27479534_1-20 | leader |
| | | NO.: 58 | | | sequence |
| HG1018308 | | SEQ. ID. | | 27479535:27479534_1-26 | leader |
| | | NO.: 59 | | | sequence |
| HG1018309 | | SEQ. ID. | | 27479535:27479534_1-21 | leader |
| | | NO.: 60 | | | sequence |
| HG1018310 | | SEQ. ID. | | 27479535:27479534_1-23 | leader |
| | | NO.: 61 | | | sequence |
| HG1018312 | | SEQ. ID. | | 37182960:37182959_1-24 | HMM_SP |
| | | NO.: 62 | | | leader |
| | | | | | sequence |
| HG1018313 | | SEQ. ID. | | 37182960:37182959_1-19 | leader |
| | | NO.: 63 | | | sequence |
| HG1018314 | | SEQ. ID. | | 37182960:37182959_1-22 | leader |
| | | NO.: 64 | | | sequence |
| HG1018315 | | SEQ. ID. | | 37182960:37182959_1-20 | leader |
| | | NO.: 65 | | | sequence |
| HG1018316 | | SEQ. ID. | | 37182960:37182959_1-26 | leader |
| | | NO.: 66 | | | sequence |
| HG1018317 | | SEQ. ID. | | 37182960:37182959_1-21 | leader |
| | | NO.: 67 | | | sequence |
| HG1018319 | | SEQ. ID. | | 7437388:1208426_1-24 | HMM_SP |
| | | NO.: 68 | | | leader |
| | | | | | sequence |
| HG1018320 | | SEQ. ID. | | 7437388:1208426_1-23 | leader |
| | | NO.: 69 | | | sequence |
| HG1018322 | | SEQ. ID. | | NP_000286:NM_000295_1-24 | HMM_SP |
| | | NO.: 70 | | | leader |
| | | | | | sequence |
| HG1018323 | | SEQ. ID. | | NP_000286:NM_000295_1-18 | leader |
| | | NO.: 71 | | | sequence |
| HG1018324 | | SEQ. ID. | | NP_000286:NM_000295_1-23 | leader |
| | | NO.: 72 | | | sequence |
| HG1018325 | | SEQ. ID. | | NP_000286:NM_000295_1-17 | leader |
| | | NO.: 73 | | | sequence |
| HG1018327 | | SEQ. ID. | | NP_000396:NM_000405_1-23 | HMM_SP |
| | | NO.: 74 | | | leader |
| | | | | | sequence |
| HG1018328 | | SEQ. ID. | | NP_000396:NM_000405_1-18 | leader |
| | | NO.: 75 | | | sequence |
| HG1018329 | | SEQ. ID. | | NP_000396:NM_000405_1-25 | leader |
| | | NO.: 76 | | | sequence |
| HG1018330 | | SEQ. ID. | | NP_000396:NM_000405_1-20 | leader |
| | | NO.: 77 | | | sequence |
| HG1018331 | | SEQ. ID. | | NP_000396:NM_000405_1-21 | leader |
| | | NO.: 78 | | | sequence |
| HG1018333 | | SEQ. ID. | | NP_000495:NM_000504_1-23 | HMM_SP |
| | | NO.: 79 | | | leader |
| | | | | | sequence |
| HG1018334 | | SEQ. ID. | | NP_000495:NM_000504_1-19 | leader |
| | | NO.: 80 | | | sequence |
| HG1018335 | | SEQ. ID. | | NP_000495:NM_000504_1-20 | leader |
| | | NO.: 81 | | | sequence |
| HG1018336 | | SEQ. ID. | | NP_000495:NM_000504_1-15 | leader |
| | | NO.: 82 | | | sequence |
| HG1018337 | | SEQ. ID. | | NP_000495:NM_000504_1-21 | leader |
| | | NO.: 83 | | | sequence |
| HG1018338 | | SEQ. ID. | | NP_000495:NM_000504_1-17 | leader |
| | | NO.: 84 | | | sequence |
| HG1018340 | | SEQ. ID. | | NP_000573:NM_000582_1-18 | HMM_SP |
| | | NO.: 85 | | | leader |
| | | | | | sequence |
| HG1018341 | | SEQ. ID. | | NP_000573:NM_000582_1-16 | leader |
| | | NO.: 86 | | | sequence |
| HG1018342 | | SEQ. ID. | | NP_000573:NM_000582_1-15 | leader |
| | | NO.: 87 | | | sequence |
| HG1018344 | | SEQ. ID. | | NP_000574:NM_000583_1-16 | HMM_SP |
| | | NO.: 88 | | | leader |
| | | | | | sequence |
| HG1018345 | | SEQ. ID. | | NP_000574:NM_000583_1-14 | leader |
| | | NO.: 89 | | | sequence |
| HG1018347 | | SEQ. ID. | | NP_000591:NM_000600_1-25 | HMM_SP |
| | | NO.: 90 | | | leader |
| | | | | | sequence |
| HG1018348 | | SEQ. ID. | | NP_000591:NM_000600_1-24 | leader |
| | | NO.: 91 | | | sequence |
| HG1018349 | | SEQ. ID. | | NP_000591:NM_000600_1-27 | leader |
| | | NO.: 92 | | | sequence |
| H01018351 | | SEQ. ID. | | NP_000598:NM_000607_1-18 | HMM_SP |
| | | NO.: 93 | | | leader |
| | | | | | sequence |
| HG1018353 | | SEQ. ID. | | NP_000604:NM_000613_1-19 | leader |
| | | NO.: 94 | | | sequence |
| HG1018354 | | SEQ. ID. | | NP_000604:NM_000613_1-25 | leader |
| | | NO.: 95 | | | sequence |
| HG1018355 | | SEQ. ID. | | NP_000604:NM_000613_1-21 | leader |
| | | NO.: 96 | | | sequence |
| HG1018356 | | SEQ. ID. | | NP_000604:NM_000613_1-23 | leader |
| | | NO.: 97 | | | sequence |
| HG1018357 | | SEQ. ID. | | NP_000604:NM_000613_1-31 | leader |
| | | NO.: 98 | | | sequence |
| HG1018359 | | SEQ. ID. | | NP_000726:NM_000735_1-26 | HMM_SP |
| | | NO.: 99 | | | leader |
| | | | | | sequence |
| HG1018360 | | SEQ. ID. | | NP_000726:NM_000735_1-24 | leader |
| | | NO.: 100 | | | sequence |
| HG1018362 | | SEQ. ID. | | NP_000884:NM_000893_1-18 | HMM_SP |
| | | NO.: 101 | | | leader |
| | | | | | sequence |
| HG1018363 | | SEQ. ID. | | NP_000884:NM_000893_1-19 | leader |
| | | NO.: 102 | | | sequence |
| HG1018364 | | SEQ. ID. | | NP_000884:NM_000893_1-16 | leader |
| | | NO.: 103 | | | sequence |
| HG1018365 | | SEQ. ID. | | NP_000884:NM_000893_1-23 | leader |
| | | NO.: 104 | | | sequence |
| HG1018367 | | SEQ. ID. | | NP_000909:NM_000918_1-17 | HMM_SP |
| | | NO.: 105 | | | leader |
| | | | | | sequence |
| HG1018369 | | SEQ. ID. | | NP_000930:NM_000939_1-23 | HMM_SP |
| | | NO.: 106 | | | leader |
| | | | | | sequence |
| HG1018370 | | SEQ. ID. | | NP_000930:NM_000939_1-26 | leader |
| | | NO.: 107 | | | sequence |
| HG1018372 | | SEQ. ID. | | NP_000945:NM_000954_1-23 | HMM_SP |
| | | NO.: 108 | | | leader |
| | | | | | sequence |
| HG1018373 | | SEQ. ID. | | NP_000945:NM_000954_1-22 | leader |
| | | NO.: 109 | | | sequence |
| HG1018374 | | SEQ. ID. | | NP_000945:NM_000954_1-18 | leader |
| | | NO.: 110 | | | sequence |
| HG1018376 | | SEQ. ID. | | NP_001176:NM_001185_1-18 | leader |
| | | NO.: 111 | | | sequence |
| HG1018377 | | SEQ. ID. | | NP_001176:NM_001185_1-20 | leader |
| | | NO.: 112 | | | sequence |
| HG1018378 | | SEQ. ID. | | NP_001176:NM_001185_1-21 | leader |
| | | NO.: 113 | | | sequence |
| HG1018379 | | SEQ. ID. | | NP_001176:NM_001185_1-17 | leader |
| | | NO.: 114 | | | sequence |
| HG1018381 | | SEQ. ID. | | NP_001266:NM_001275_1-18 | HMM_SP |
| | | NO.: 115 | | | leader |
| | | | | | sequence |
| HG1018382 | | SEQ. ID. | | NP_001266:NM_001275_1-15 | leader |
| | | NO.: 116 | | | sequence |
| HG1018383 | | SEQ. ID. | | NP_001266:NM_001275_1-14 | leader |
| | | NO.: 117 | | | sequence |
| HG1018385 | | SEQ. ID. | | NP_001314:NM_001323_1-26 | HMM_SP |
| | | NO.: 118 | | | leader |
| | | | | | sequence |
| HG1018386 | | SEQ. ID. | | NP_001314:NM_001323_1-18 | leader |
| | | NO.: 119 | | | sequence |
| HG1018387 | | SEQ. ID. | | NP_001314:NM_001323_1-20 | leader |
| | | NO.: 120 | | | sequence |
| HG1018388 | | SEQ. ID. | | NP_001314:NM_001323_1-28 | leader |
| | | NO.: 121 | | | sequence |
| HG1018389 | | SEQ. ID. | | NP_001314:NM_001323_1-21 | leader |
| | | NO.: 122 | | | sequence |
| HG1018390 | | SEQ. ID. | | NP_001314:NM_001323_1-23 | leader |
| | | NO.: 123 | | | sequence |
| HG1018392 | | SEQ. ID. | | NP_001822:NM_001831_1-22 | leader |
| | | NO.: 124 | | | sequence |
| HG1018393 | | SEQ. ID. | | NP_001822:NM_001831_1-18 | leader |
| | | NO.: 125 | | | sequence |
| HG1018394 | | SEQ. ID. | | NP_001822:NM_001831_1-14 | leader |
| | | NO.: 126 | | | sequence |
| HG1018396 | | SEQ. ID. | | NP_002206:NM_002215_1-24 | leader |
| | | NO.: 127 | | | sequence |
| HG1018397 | | SEQ. ID. | | NP_002206:NM_002215_1-29 | leader |
| | | NO.: 128 | | | sequence |
| HG1018398 | | SEQ. ID. | | NP_002206:NM_002215_1-30 | leader |
| | | NO.: 129 | | | sequence |
| HG1018399 | | SEQ. ID. | | NP_002206:NM_002215_1-23 | leader |
| | | NO.: 130 | | | sequence |
| HG1018400 | | SEQ. ID. | | NP_002206:NM_002215_1-31 | leader |
| | | NO.: 131 | | | sequence |
| HG1018402 | | SEQ. ID. | | NP_002300:NM_002309_1-22 | HMM_SP |
| | | NO.: 132 | | | leader |
| | | | | | sequence |
| HG1018403 | | SEQ. ID. | | NP_002300:NM_002309_1-23 | leader |
| | | NO.: 133 | | | sequence |
| HG1018405 | | SEQ. ID. | | NP_002336:NM_002345_1-18 | HMM_SP |
| | | NO.: 134 | | | leader |
| | | | | | sequence |
| HG1018406 | | SEQ. ID. | | NP_002336:NM_002345_1-15 | leader |
| | | NO.: 135 | | | sequence |
| HG1018407 | | SEQ. ID. | | NP_002336:NM_002345_1-17 | leader |
| | | NO.: 136 | | | sequence |
| HG1018408 | | SEQ. ID. | | NP_002336:NM_002345_1-14 | leader |
| | | NO.: 137 | | | sequence |
| HG1018410 | | SEQ. ID. | | NP_002402:NM_002411_1-18 | HMM_SP |
| | | NO.: 138 | | | leader |
| | | | | | sequence |
| HG1018412 | | SEQ. ID. | | NP_002505:NM_002514_1-30 | HMM_SP |
| | | NO.: 139 | | | leader |
| | | | | | sequence |
| HG1018413 | | SEQ. ID. | | NP_002505:NM_002514_1-32 | leader |
| | | NO.: 140 | | | sequence |
| HG1018414 | | SEQ. ID. | | NP_002505:NM_002514_1-28 | leader |
| | | NO.: 141 | | | sequence |
| HG1018415 | | SEQ. ID. | | NP_002505:NM_002514_1-27 | leader |
| | | NO.: 142 | | | sequence |
| HG1018416 | | SEQ. ID. | | NP_002505:NM_002514_1-31 | leader |
| | | NO.: 143 | | | sequence |
| HG1018418 | | SEQ. ID. | | NP_002892:NM_002901_1-26 | HMM_SP |
| | | NO.: 144 | | | leader |
| | | | | | sequence |
| HG1018419 | | SEQ. ID. | | NP_002892:NM_002901_1-22 | leader |
| | | NO.: 145 | | | sequence |
| HG1018420 | | SEQ. ID. | | NP_002892:NM_002901_1-29 | leader |
| | | NO.: 146 | | | sequence |
| HG1018421 | | SEQ. ID. | | NP_002892:NM_002901_1-24 | leader |
| | | NO.: 147 | | | sequence |
| HG1018422 | | SEQ. ID. | | NP_002892:NM_002901_1-23 | leader |
| | | NO.: 148 | | | sequence |
| HG1018424 | | SEQ. ID. | | NP_002893:NM_002902_1-25 | HMM_SP |
| | | NO.: 149 | | | leader |
| | | | | | sequence |
| HG1018425 | | SEQ. ID. | | NP_002893:NM_002902_1-19 | leader |
| | | NO.: 150 | | | sequence |
| HG1018426 | | SEQ. ID. | | NP_002893:NM_002902_1-22 | leader |
| | | NO.: 151 | | | sequence |
| HG1018427 | | SEQ. ID. | | NP_002893:NM_002902_1-18 | leader |
| | | NO.: 152 | | | sequence |
| HG1018428 | | SEQ. ID. | | NP_002893:NM_002902_1-20 | leader |
| | | NO.: 153 | | | sequence |
| HG1018429 | | SEQ. ID. | | NP_002893:NM_002902_1-21 | leader |
| | | NO.: 154 | | | sequence |
| HG1018430 | | SEQ. ID. | | NP_002893:NM_002902_1-23 | leader |
| | | NO.: 155 | | | sequence |
| HG1018432 | | SEQ. ID. | | NP_005133:NM_005142_1-19 | HMM_SP |
| | | NO.: 156 | | | leader |
| | | | | | sequence |
| HG1018433 | | SEQ. ID. | | NP_005133:NM_005142_1-18 | leader |
| | | NO.: 157 | | | sequence |
| HG1018434 | | SEQ. ID. | | NP_005133:NM_005142_1-20 | leader |
| | | NO.: 158 | | | sequence |
| HG1018435 | | SEQ. ID. | | NP_005133:NM_005142_1-24 | leader |
| | | NO.: 159 | | | sequence |
| HG1018436 | | SEQ. ID. | | NP_005133:NM_005142_1-16 | leader |
| | | NO.: 160 | | | sequence |
| HG1018437 | | SEQ. ID. | | NP_005133:NM_005142_1-17 | leader |
| | | NO.: 161 | | | sequence |
| HG1018438 | | SEQ. ID. | | NP_005133:NM_005142_1-14 | leader |
| | | NO.: 162 | | | sequence |
| HG1018440 | | SEQ. ID. | | NP_005445:NM_005454_1-17 | HMM_SP |
| | | NO.: 163 | | | leader |
| | | | | | sequence |
| HG1018442 | | SEQ. ID. | | NP_005555:NM_005564_1-18 | HMM_SP |
| | | NO.: 164 | | | leader |
| | | | | | sequence |
| HG1018443 | | SEQ. ID. | | NP_005555:NM_005564_1-20 | leader |
| | | NO.: 165 | | | sequence |
| HG1018444 | | SEQ. ID. | | NP_005555:NM_005564_1-15 | leader |
| | | NO.: 166 | | | sequence |
| HG1018446 | | SEQ. ID. | | NP_005690:NM_005699_1-29 | HMM_SP |
| | | NO.: 167 | | | leader |
| | | | | | sequence |
| HG1018447 | | SEQ. ID. | | NP_005690:NM_005699_1-24 | leader |
| | | NO.: 168 | | | sequence |
| HG1018448 | | SEQ. ID. | | NP_005690:NM_005699_1-28 | leader |
| | | NO.: 169 | | | sequence |
| HG1018450 | | SEQ. ID. | | NP_006560:NM_006569_1-19 | HMM_SP |
| | | NO.: 170 | | | leader |
| | | | | | sequence |
| HG1018451 | | SEQ. ID. | | NP_006560:NM_006569_1- | leader |
| | | NO.: 171 | | 18 | sequence |
| HG1018452 | | SEQ. ID. | | NP_006560:NM_006569_1- | leader |
| | | NO.: 172 | | 21 | sequence |
| HG1018454 | | SEQ. ID. | | NP_006856:NM_006865_1- | HMM_SP |
| | | NO.: 173 | | 15 | leader |
| | | | | | sequence |
| HG1018456 | | SEQ. ID. | | NP_036577:NM_012445_1- | HMM_SP |
| | | NO.: 174 | | 26 | leader |
| | | | | | sequence |
| HG1018457 | | SEQ. ID. | | NP_036577:NM_012445_1- | leader |
| | | NO.: 175 | | 25 | sequence |
| HG1018458 | | SEQ. ID. | | NP_036577:NM_012445_1- | leader |
| | | NO.: 176 | | 24 | sequence |
| HG1018459 | | SEQ. ID. | | NP_036577:NM_012445_1- | leader |
| | | NO.: 177 | | 28 | sequence |
| HG1018461 | | SEQ. ID. | | NP_055070:NM_014255_1- | HMM_SP |
| | | NO.: 178 | | 20 | leader |
| | | | | | sequence |
| HG1018462 | | SEQ. ID. | | NP_055070:NM_014255_1- | leader |
| | | NO.: 179 | | 18 | sequence |
| HG1018463 | | SEQ. ID. | | NP_055070:NM_014255_1- | leader |
| | | NO.: 180 | | 16 | sequence |
| HG1018465 | | SEQ. ID. | | NP_055582:NM_014767_1- | HMM_SP |
| | | NO.: 181 | | 24 | leader |
| | | | | | sequence |
| HG1018466 | | SEQ. ID. | | NP_055582:NM_014767_1- | leader |
| | | NO.: 182 | | 19 | sequence |
| HG1018467 | | SEQ. ID. | | NP_055582:NM_014767_1- | leader |
| | | NO.: 183 | | 22 | sequence |
| HG1018468 | | SEQ. ID. | | NP_055582:NM_014767_1- | leader |
| | | NO.: 184 | | 20 | sequence |
| HG1018469 | | SEQ. ID. | | NP_055582:NM_014767_1- | leader |
| | | NO.: 185 | | 26 | sequence |
| HG1018470 | | SEQ. ID. | | NP_055582:NM_014767_1- | leader |
| | | NO.: 186 | | 21 | sequence |
| HG1018472 | | SEQ. ID. | | NP_055697:NM_014882_1- | HMM_SP |
| | | NO.: 187 | | 18 | leader |
| | | | | | sequence |
| HG1018474 | | SEQ. ID. | | NP_056965:NM_015881_1- | HMM_SP |
| | | NO.: 188 | | 18 | leader |
| | | | | | sequence |
| HG1018475 | | SEQ. ID. | | NP_056965:NM_015881_1- | leader |
| | | NO.: 189 | | 19 | sequence |
| HG1018476 | | SEQ. ID. | | NP_056965:NM_015881_1- | leader |
| | | NO.: 190 | | 22 | sequence |
| HG1018477 | | SEQ. ID. | | NP_056965:NM_015881_1- | leader |
| | | NO.: 191 | | 16 | sequence |
| HG1018478 | | SEQ. ID. | | NP_056965:NM_015881_1- | leader |
| | | NO.: 192 | | 21 | sequence |
| HG1018480 | | SEQ. ID. | | NP_057603:NM_016519_1- | leader |
| | | NO.: 193 | | 26 | sequence |
| HG1018481 | | SEQ. ID. | | NP_057603:NM_016519_1- | leader |
| | | NO.: 194 | | 28 | sequence |
| HG1018483 | | SEQ. ID. | | NP_149439:NM_033183_1- | HMM_SP |
| | | NO.: 195 | | 18 | leader |
| | | | | | sequence |
| HG1018484 | | SEQ. ID. | | NP_149439:NM_033183_1- | leader |
| | | NO.: 196 | | 20 | sequence |
| HG1018485 | | SEQ. ID. | | NP_149439:NM_033183_1- | leader |
| | | NO.: 197 | | 16 | sequence |
| HG1018487 | | SEQ. ID. | | NP_644808:NM_139279_1- | leader |
| | | NO.: 198 | | 18 | sequence |
| HG1018488 | | SEQ. ID. | | NP_644808:NM_139279_1- | leader |
| | | NO.: 199 | | 20 | sequence |
| HG1018489 | | SEQ. ID. | | NP_644808:NM_139279_1- | leader |
| | | NO.: 200 | | 26 | sequence |
| HG1018490 | | SEQ. ID. | | NP_644808:NM_139279_1- | leader |
| | | NO.: 201 | | 23 | sequence |
| HG1018492 | | SEQ. ID. | | NP_660295:NM_145252_1- | leader |
| | | NO.: 202 | | 13 | sequence |
| HG1018493 | | SEQ. ID. | | NP_660295:NM_145252_1- | leader |
| | | NO.: 203 | | 16 | sequence |
| HG1018494 | | SEQ. ID. | | NP_660295:NM_145252_1- | leader |
| | | NO.: 204 | | 14 | sequence |
| HG1018495 | | SEQ. ID. | | NP_660295:NM_145252_1- | leader |
| | | NO.: 205 | | 17 | sequence |
| HG1018497 | | SEQ. ID. | | NP_689534:NM_152321_1- | HMM_SP |
| | | NO.: 206 | | 25 | leader |
| | | | | | sequence |
| HG1018498 | | SEQ. ID. | | NP_689534:NM_152321_1- | leader |
| | | NO.: 207 | | 21 | sequence |
| HG1018500 | | SEQ. ID. | | NP_689848:NM_152635_1- | HMM_SP |
| | | NO.: 208 | | 18 | leader |
| | | | | | sequence |
| HG1018501 | | SEQ. ID. | | NP_689848:NM_152635_1- | leader |
| | | NO.: 209 | | 16 | sequence |
| HG1018502 | | SEQ. ID. | | NP_689848:NM_152635_1- | leader |
| | | NO.: 210 | | 15 | sequence |
| HG1018504 | | SEQ. ID. | | NP_689968:NM_152755_1- | HMM_SP |
| | | NO.: 211 | | 21 | leader |
| | | | | | sequence |
| HG1018506 | | SEQ. ID. | | NP_766630:NM_173042_1- | HMM_SP |
| | | NO.: 212 | | 29 | leader |
| | | | | | sequence |
| HG1018507 | | SEQ. ID. | | NP_766630:NM_173042_1- | leader |
| | | NO.: 213 | | 24 | sequence |
| HG1018508 | | SEQ. ID. | | NP_766630:NM_173042_1- | leader |
| | | NO.: 214 | | 28 | sequence |
| HG1018510 | | SEQ. ID. | | NP_776214:NM_173842_1- | HMM_SP |
| | | NO.: 215 | | 23 | leader |
| | | | | | sequence |
| HG1018511 | | SEQ. ID. | | NP_776214:NM_173842_1- | leader |
| | | NO.: 216 | | 25 | sequence |
| HG1018513 | | SEQ. ID. | | NP_783165:NM_175575_1- | HMM_SP |
| | | NO.: 217 | | 32 | leader |
| | | | | | sequence |
| HG1018514 | | SEQ. ID. | | NP_783165:NM_175575_1- | leader |
| | | NO.: 218 | | 34 | sequence |
| HG1018515 | | SEQ. ID. | | NP_783165:NM_175575_1- | leader |
| | | NO.: 219 | | 29 | sequence |
| HG1018516 | | SEQ. ID. | | NP_783165:NM_175575_1- | leader |
| | | NO.: 220 | | 30 | sequence |
| HG1018517 | | SEQ. ID. | | NP_783165:NM_175575_1- | leader |
| | | NO.: 221 | | 27 | sequence |
| HG1018857 | | SEQ. ID. | | 27482680:27482679_1-26 | HMM_SP |
| | | NO.: 222 | | | leader |
| | | | | | sequence |
| HG1018858 | | SEQ. ID. | | 27482680:27482679_1-24 | leader |
| | | NO.: 223 | | | sequence |

TABLE 2
Characterization of Splice Variants

| | | | | | | | Top | |
|---|---|---|---|---|---|---|---|---|
| | | | Top | | | | Human | |
| | | | Human | | Top | | Hit % | % ID |
| | | Pred | Hit | | Hum | | ID over | over |
| | | Prot | Access | | Hit | Match | Query | Hum |
| FP ID | Clone ID | Len | ID | Top Human Hit Annotation | Len | Len | Len | Hit Len |
| HG1015090 | CLN00493987_5pv1.a | 179 | gi\|5803086\| | interleukin 24 isoform 1 | 206 | 179 | 100% | 87% |
| | | | ref\| | precursor; melanoma | | | | |
| | | | NP_006841.1\| | differentiation association | | | | |
| | | | | protein 7; suppression of | | | | |
| | | | | tumorigenicity 16 (melanoma | | | | |
| | | | | differentiation) [Homo | | | | |
| | | | | sapiens] | | | | |
| HG1015091 | NP_006841: | 206 | gi\|5803086\| | interleukin 24 isoform 1 | 206 | 206 | 100% | 100% |
| | NM_006850 | | ref\| | precursor; melanoma | | | | |
| | | | NP_006841.1\| | differentiation association | | | | |
| | | | | protein 7; suppression of | | | | |
| | | | | tumorigenicity 16 (melanoma | | | | |
| | | | | differentiation) [Homo | | | | |
| | | | | sapiens] | | | | |
| HG1015092 | CLN00453866_5pv1.a | 126 | gi\|5803086\| | interleukin 24 isoform 1 | 207 | 126 | 100% | 61% |
| | | | ref\| | precursor; melanoma | | | | |
| | | | NP_006841.1\| | differentiation association | | | | |
| | | | | protein 7; suppression of | | | | |
| | | | | tumorigenicity 16 (melanoma | | | | |
| | | | | differentiation) [Homo | | | | |
| | | | | sapiens] | | | | |
| HG1015093 | NP_006841: | 14 | gi\|5803086\| | interleukin 24 isoform 1 | 207 | 14 | 100% | 7% |
| | NM_006850_exon1 | | ref\| | precursor; melanoma | | | | |
| | | | NP_006841.1\| | differentiation association | | | | |
| | | | | protein 7; suppression of | | | | |
| | | | | tumorigenicity 16 (melanoma | | | | |
| | | | | differentiation) [Homo | | | | |
| | | | | sapiens] | | | | |
| HG1015094 | NP_006841: | 53 | gi\|5803086\| | interleukin 24 isoform 1 | 207 | 53 | 100% | 26% |
| | NM_006850_exon4 | | ref\| | precursor; melanoma | | | | |
| | | | NP_006841.1\| | differentiation association | | | | |
| | | | | protein 7; suppression of | | | | |
| | | | | tumorigenicity 16 (melanoma | | | | |
| | | | | differentiation) [Homo | | | | |
| | | | | sapiens] | | | | |
| HG1014901 | CLN00108891_5pv1.a | 235 | gi\|4507593\| | tumor necrosis factor (ligand) | 281 | 235 | 100% | 84% |
| | | | ref\| | superfamily, member 10; Apo- | | | | |
| | | | NP_003801.1\| | 2 ligand; TNF-related | | | | |
| | | | | apoptosis inducing ligand | | | | |
| | | | | TRAIL [Homo sapiens] | | | | |
| HG1019036 | CLN00108891_frag1 | 190 | gi\|4507593\| | tumor necrosis factor (ligand) | 281 | 190 | 100% | 68% |
| | | | ref\| | superfamily, member 10; Apo- | | | | |
| | | | NP_003801.1\| | 2 ligand; TNF-related | | | | |
| | | | | apoptosis inducing ligand | | | | |
| | | | | TRAIL [Homo sapiens] | | | | |
| HG1019037 | CLN00108891_frag2 | 196 | gi\|4507593\| | tumor necrosis factor (ligand) | 281 | 191 | 97% | 68% |
| | | | ref\| | superfamily, member 10; Apo- | | | | |
| | | | NP_003801.1\| | 2 ligand; TNF-related | | | | |
| | | | | apoptosis inducing ligand | | | | |
| | | | | TRAIL [Homo sapiens] | | | | |
| HG1019038 | NP_003801: | 281 | gi\|4507593\| | tumor necrosis factor (ligand) | 281 | 281 | 100% | 100% |
| | NM_003810 | | ref\| | superfamily, member 10; Apo- | | | | |
| | | | NP_003801.1\| | 2 ligand; TNF-related | | | | |
| | | | | apoptosis inducing ligand | | | | |
| | | | | TRAIL [Homo sapiens] | | | | |
| HG1019039 | NP_003801: | 168 | gi\|4507593\| | tumor necrosis factor (ligand) | 281 | 168 | 100% | 60% |
| | NM_003810_frag1 | | ref\| | superfamily, member 10; Apo- | | | | |
| | | | NP_003801.1\| | 2 ligand; TNF-related | | | | |
| | | | | apoptosis inducing ligand | | | | |
| | | | | TRAIL [Homo sapiens] | | | | |

TABLE 3
Splice Variant Coordinates

| | | | | Pred | | | Alt Mat | | | | | |
|---|---|---|---|---|---|---|---|---|---|---|---|---|
| | | | | Prot | Tree | Mat Prot | Prot | Sig Pep | | TM | Non-TM | |
| FP ID | Clone ID | Cluster | Class | Len | vote | Coords | Coords | Coords | TM | Coords | Coords | Pfam |
| HG1015090 | CLN00493987_5pv1.a | | | 179 | 0.99 | (22-179) | | (3-21) | 0 | | (1-179) | no_pfam |
| HG1015091 | NP_006841: | 204200 | SEC | 206 | 1 | (24-206) | (1-206) | | 0 | | (1-206) | no_pfam |
| | NM_006850 | | | | | | | | | | | |
| HG1015092 | CLN00453866_5pv1.a | | | 126 | 1 | (22-126) | | (3-21) | 0 | | (1-126) | no_pfam |
| HG1015093 | NP_006841: | 204200 | SEC | 14 | | (1-14) | | (1-14) | 0 | | (1-14) | no_pfam |
| | NM_006850_exon1 | | | | | | | | | | | |
| HG1015094 | NP_006841: | 204200 | SEC | 53 | 0.01 | (1-53) | | | 0 | | (1-53) | no_pfam |
| | NM_006850_exon4 | | | | | | | | | | | |
| HG1014901 | CLN00108891_5pv1.a | | | 235 | 0.73 | (34-235) | (35-235) | (3-33) | 1 | (17-39) | (1-16) | TNF |
| | | | | | | | (31-235) | | | | (40-235) | |
| | | | | | | | (33-235) | | | | | |
| HG1019036 | CLN00108891_frag1 | | | 190 | 0 | (1-190) | | | 0 | | (1-190) | TNF |
| HG1019037 | CLN00108891_frag2 | | | 196 | 0 | (1-196) | | | 0 | | (1-196) | TNF |
| HG1019038 | NP_003801: | 183109 | STM | 281 | 0.64 | (34-281) | (35-281) | (3-33) | 1 | (15-37) | (1-14) | TNF |
| | NM_003810 | | Type II | | | | (31-281) | | | | (38-281) | |
| | | | membrane | | | | (33-281) | | | | | |
| HG1019039 | NP_003801: | 183109 | STM | 168 | 0 | (1-168) | | | 0 | | (1-168) | TNF |
| | NM_003810_frag1 | | Type II | | | | | | | | | |
| | | | membrane | | | | | | | | | |

TABLE 4
Pfam Domains

| FP ID | Source ID | Pfam | Coords |
|---|---|---|---|
| HG1014901 | CLN00108891_5pv1.a | TNF | (107-234) |
| HG1019036 | CLN00108891_frag1 | TNF | (62-189) |
| HG1019037 | CLN00108891_frag2 | TNF | (68-195) |
| HG1019038 | NP_003801:NM_003810 | TNF | (153-280) |
| HG1019039 | NP_003801:NM_003810_frag1 | TNF | (40-167) |

TABLE 5
Characterization of Secretory Leaders

| FP ID | Source ID | Annotation |
|---|---|---|
| HG1018268 | 112907:21594845_1-17 | Alpha-2-antiplasmin precursor (Alpha-2-plasmin inhibitor) |
| HG1018269 | 112907:21594845_1-13 | Alpha-2-antiplasmin precursor (Alpha-2-plasmin inhibitor) |
| HG1018270 | 112907:21594845_1-19 | Alpha-2-antiplasmin precursor (Alpha-2-plasmin inhibitor) |
| HG1018271 | 112907:21594845_1-16 | Alpha-2-antiplasmin precursor (Alpha-2-plasmin inhibitor) |
| HG1018272 | 112907:21594845_1-15 | Alpha-2-antiplasmin precursor (Alpha-2-plasmin inhibitor) |
| HG1018274 | 13325208:13325207_1-30 | Trinucleotide repeat containing 5 [Homo sapiens] |
| HG1018275 | 13325208:13325207_1-25 | Trinucleotide repeat containing 5 [Homo sapiens] |
| HG1018276 | 13325208:13325207_1-33 | Trinucleotide repeat containing 5 [Homo sapiens] |
| HG1018277 | 13325208:13325207_1-24 | Trinucleotide repeat containing 5 [Homo sapiens] |
| HG1018278 | 13325208:13325207_1-26 | Trinucleotide repeat containing 5 [Homo sapiens] |
| HG1018279 | 13325208:13325207_1-32 | Trinucleotide repeat containing 5 [Homo sapiens] |
| HG1018280 | 13325208:13325207_1-27 | Trinucleotide repeat containing 5 [Homo sapiens] |
| HG1018281 | 13325208:13325207_1-23 | Trinucleotide repeat containing 5 [Homo sapiens] |
| HG1018282 | 13325208:13325207_1-35 | Trinucleotide repeat containing 5 [Homo sapiens] |
| HG1018284 | 13938307:13938306_1-24 | ARMET protein [Homo sapiens] |
| HG1018285 | 13938307:13938306_1-21 | ARMET protein [Homo sapiens] |
| HG1018287 | 14718453:14718452_1-19 | calumenin [Homo sapiens] |
| HG1018288 | 14718453:14718452_1-15 | calumenin [Homo sapiens] |
| HG1018289 | 14718453:14718452_1-17 | calumenin [Homo sapiens] |
| HG1018291 | 15929966:15929965_1-23 | COL9A1 protein [Homo sapiens] |
| HG1018293 | 16356651:16356650_1-21 | NBL1 [Homo sapiens] |
| HG1018294 | 16356651:16356650_1-17 | NBL1 [Homo sapiens] |
| HG1018296 | 18204192:18204191_1-19 | PACAP protein [Homo sapiens] |
| HG1018297 | 18204192:18204191_1-22 | PACAP protein [Homo sapiens] |
| HG1018298 | 18204192:18204191_1-18 | PACAP protein [Homo sapiens] |
| HG1018299 | 18204192:18204191_1-16 | PACAP protein [Homo sapiens] |
| HG1018300 | 18204192:18204191_1-14 | PACAP protein [Homo sapiens] |
| HG1018302 | 23503038:15778555_1-20 | Alpha-1B-glycoprotein precursor (Alpha-1-B glycoprotein) |
| HG1018303 | 23503038:15778555_1-16 | Alpha-1B-glycoprotein precursor (Alpha-1-B glycoprotein) |
| HG1018304 | 23503038:15778555_1-21 | Alpha-1B-glycoprotein precursor (Alpha-1-B glycoprotein) |
| HG1018306 | 27479535:27479534_1-24 | similar to Brain-specific angiogenesis inhibitor 2 precursor |
| | | [Homo sapiens] |
| HG1018307 | 27479535:27479534_1-20 | similar to Brain-specific angiogenesis inhibitor 2 precursor |
| | | [Homo sapiens] |
| HG1018308 | 27479535:27479534_1-26 | similar to Brain-specific angiogenesis inhibitor 2 precursor |
| | | [Homo sapiens] |
| HG1018309 | 27479535:27479534_1-21 | similar to Brain-specific angiogenesis inhibitor 2 precursor |
| | | [Homo sapiens] |
| HG1018310 | 27479535:27479534_1-23 | similar to Brain-specific angiogenesis inhibitor 2 precursor |
| | | [Homo sapiens] |
| HG1018312 | 37182960:37182959_1-24 | SPOCK2 [Homo sapiens] |
| HG1018313 | 37182960:37182959_1-19 | SPOGK2 [Homo sapiens] |
| HG1018314 | 37182960:37182959_1-22 | SPOCK2 [Homo sapiens] |
| HG1018315 | 37182960:37182959_1-20 | SPOCK2 [Homo sapiens] |
| HG1018316 | 37182960:37182959_1-26 | SPOCK2 [Homo sapiens] |
| HG1018317 | 37182960:37182959_1-21 | SPOCK2 [Homo sapiens] |
| HG1018319 | 7437388:1208426_1-24 | protein disulfide-isomerase (EC 5341) ER60 precursor - |
| | | human |
| HG1018320 | 7437388:1208426_1-23 | protein disulfide-isomerase (EC 5341) ER60 precursor - |
| | | human |
| HG1018322 | NP_000286:NM_000295_1-24 | serine (or cysteine) proteinase inhibitor, clade A (alpha-1 |
| HG1018323 | NP_000286:NM_000295_1-18 | serine (or cysteine) proteinase inhibitor, clade A (alpha-1 |
| HG1018324 | NP_000286:NM_000295_1-23 | serine (or cysteine) proteinase inhibitor, clade A (alpha-1 |
| HG1018325 | NP_000286:NM_000295_1-17 | serine (or cysteine) proteinase inhibitor, clade A (alpha-1 |
| HG1018327 | NP_000396:NM_000405_1-23 | GM2 ganglioside activator precursor [Homo sapiens] |
| HG1018328 | NP_000396:NM_000405_1-18 | GM2 ganglioside activator precursor [Homo sapiens] |
| HG1018329 | NP_000396:NM_000405_1-25 | GM2 ganglioside activator precursor [Homo sapiens] |
| HG1018330 | NP_000396:NM_000405_1-20 | GM2 ganglioside activator precursor [Homo sapiens] |
| HG1018331 | NP_000396:NM_000405_1-21 | GM2 ganglioside activator precursor [Homo sapiens] |
| HG1018333 | NP_000495:NM_000504_1-23 | coagulation factor X precursor [Homo sapiens] |
| HG1018334 | NP_000495:NM_000504_1-19 | coagulation factor X precursor [Homo sapiens] |
| HG1018335 | NP_000495:NM_000504_1-20 | coagulation factor X precursor [Homo sapiens] |
| HG1018336 | NP_000495:NM_000504_1-15 | coagulation factor X precursor [Homo sapiens] |
| HG1018337 | NP_000495:NM_000504_1-21 | coagulation factor X precursor [Homo sapiens] |
| HG1018338 | NP_000495:NM_000504_1-17 | coagulation factor X precursor [Homo sapiens] |
| HG1018340 | NP_000573:NM_000582_1-18 | secreted phosphoprotein 1 (osteopontin, bone sialoprotein I, |
| | | early |
| HG1018341 | NP_000573:NM_000582_1-16 | secreted phosphoprotein 1 (osteopontin, bone sialoprotein I, |
| | | early |
| HG1018342 | NP_000573:NM_000582_1-15 | secreted phosphoprotein 1 (osteopontin, bone sialoprotein I, |
| | | early |
| HG1018344 | NP_000574:NM_000583_1-16 | vitamin D-binding protein precursor [Homo sapiens] |
| HG1018345 | NP_000574:NM_000583_1-14 | vitamin D-binding protein precursor [Homo sapiens] |
| HG1018347 | NP_000591:NM_000600_1-25 | interleukin 6 (interferon, beta 2) [Homo sapiens] |
| HG1018348 | NP_000591:NM_000600_1-24 | interleukin 6 (interferon, beta 2) [Homo sapiens] |
| HG1018349 | NP_000591:NM_000600_1-27 | interleukin 6 (interferon, beta 2) [Homo sapiens] |
| HG1018351 | NP_000598:NM_000607_1-18 | orosomucoid 1 precursor [Homo sapiens] |
| HG1018353 | NP_000604:NM_000613_1-19 | hemopexin [Homo sapiens] |
| HG1018354 | NP_000604:NM_000613_1-25 | hemopexin [Homo sapiens] |
| HG1018355 | NP_000604:NM_000613_1-21 | hemopexin [Homo sapiens] |
| HG1018356 | NP_000604:NM_000613_1-23 | hemopexin [Homo sapiens] |
| HG1018357 | NP_000604:NM_000613_1-31 | hemopexin [Homo sapiens] |
| HG1018359 | NP_000726:NM_000735_1-26 | glycoprotein hormones, alpha polypeptide precursor [Homo |
| | | sapiens] |
| HG1018360 | NP_000726:NM_000735_1-24 | glycoprotein hormones, alpha polypeptide precursor [Homo |
| | | sapiens] |
| HG1018362 | NP_000884:NM_000893_1-18 | kininogen 1 [Homo sapiens] |
| HG1018363 | NP_000884:NM_000893_1-19 | kininogen 1 [Homo sapiens] |
| HG1018364 | NP_000884:NM_000893_1-16 | kininogen 1 [Homo sapiens] |
| HG1018365 | NP_000884:NM_000893_1-23 | kininogen 1 [Homo sapiens] |
| HG1018367 | NP_000909:NM_000918_1-17 | prolyl 4-hydroxylase, beta subunit [Homo sapiens] |
| HG1018369 | NP_000930:NM_000939_1-23 | proopiomelanocortin [Homo sapiens] |
| HG1018370 | NP_000930:NM_000939_1-26 | proopiomelanocortin [Homo sapiens] |
| HG1018372 | NP_000945:NM_000954_1-23 | prostaglandin D2 synthase 21 kDa [Homo sapiens] |
| HG1018373 | NP_000945:NM_000954_1-22 | prostaglandin D2 synthase 21 kDa [Homo sapiens] |
| HG1018374 | NP_000945:NM_000954_1-18 | prostaglandin D2 synthase 21 kDa [Homo sapiens] |
| HG1018376 | NP_001176:NM_001185_1-18 | alpha-2-glycoprotein 1, zinc [Homo sapiens] |
| HG1018377 | NP_001176:NM_001185_1-20 | alpha-2-glycoprotein 1, zinc [Homo sapiens] |
| HG1018378 | NP_001176:NM_001185_1-21 | alpha-2-glycoprotein 1, zinc [Homo sapiens] |
| HG1018379 | NP_001176:NM_001185_1-17 | alpha-2-glycoprotein 1, zinc [Homo sapiens] |
| HG1018381 | NP_001266:NM_001275_1-18 | chromogranin A [Homo sapiens] |
| HG1018382 | NP_001266:NM_001275_1-15 | chromogranin A [Homo sapiens] |
| HG1018383 | NP_001266:NM_001275_1-14 | chromogranin A [Homo sapiens] |
| HG1018385 | NP_001314:NM_001323_1-26 | cystatin M precursor [Homo sapiens] |
| HG1018386 | NP_001314:NM_001323_1-18 | cystatin M precursor [Homo sapiens] |
| HG1018387 | NP_001314:NM_001323_1-20 | cystatin M precursor [Homo sapiens] |
| HG1018388 | NP_001314:NM_001323_1-28 | cystatin M precursor [Homo sapiens] |
| HG1018389 | NP_001314:NM_001323_1-21 | cystatin M precursor [Homo sapiens] |
| HG1018390 | NP_001314:NM_001323_1-23 | cystatin M precursor [Homo sapiens] |
| HG1018392 | NP_001822:NM_001831_1-22 | clusterin isoform 1 [Homo sapiens] |
| HG1018393 | NP_001822:NM_001831_1-18 | clusterin isoform 1 [Homo sapiens] |
| HG1018394 | NP_001822:NM_001831_1-14 | clusterin isoform 1 [Homo sapiens] |
| HG1018396 | NP_002206:NM_002215_1-24 | inter-alpha (globulin) inhibitor H1 [Homo sapiens] |
| HG1018397 | NP_002206:NM_002215_1-29 | inter-alpha (globulin) inhibitor H1 [Homo sapiens] |
| HG1018398 | NP_002206:NM_002215_1-30 | inter-alpha (globulin) inhibitor H1 [Homo sapiens] |
| HG1018399 | NP_002206:NM_002215_1-23 | inter-alpha (globulin) inhibitor H1 [Homo sapiens] |
| HG1018400 | NP_002206:NM_002215_1-31 | inter-alpha (globulin) inhibitor H1 [Homo sapiens] |
| HG1018402 | NP_002300:NM_002309_1-22 | leukemia inhibitory factor (cholinergic differentiation factor) |
| HG1018403 | NP_002300:NM_002309_1-23 | leukemia inhibitory factor (cholinergic differentiation factor) |
| HG1018405 | NP_002336:NM_002345_1-18 | lumican [Homo sapiens] |
| HG1018406 | NP_002336:NM_002345_1-15 | lumican [Homo sapiens] |
| HG1018407 | NP_002336:NM_002345_1-17 | lumican [Homo sapiens] |
| HG1018408 | NP_002336:NM_002345_1-14 | lumican [Homo sapiens] |
| HG1018410 | NP_002402:NM_002411_1-18 | secretoglobin, family 2A, member 2 [Homo sapiens] |
| HG1018412 | NP_002505:NM_002514_1-30 | nov precursor [Homo sapiens] |
| HG1018413 | NP_002505:NM_002514_1-32 | nov precursor [Homo sapiens] |
| HG1018414 | NP_002505:NM_002514_1-28 | nov precursor [Homo sapiens] |
| HG1018415 | NP_002505:NM_002514_1-27 | nov precursor [Homo sapiens] |
| HG1018416 | NP_002505:NM_002514_1-31 | nov precursor [Homo sapiens] |
| HG1018418 | NP_002892:NM_002901_1-26 | reticulocalbin 1 precursor [Homo sapiens] |
| HG1018419 | NP_002892:NM_002901_1-22 | reticulocalbin 1 precursor [Homo sapiens] |
| HG1018420 | NP_002892:NM_002901_1-29 | reticulocalbin 1 precursor [Homo sapiens] |
| HG1018421 | NP_002892:NM_002901_1-24 | reticulocalbin 1 precursor [Homo sapiens] |
| HG1018422 | NP_002892:NM_002901_1-23 | reticulocalbin 1 precursor [Homo sapiens] |
| HG1018424 | NP_002893:NM_002902_1-25 | reticulocalbin 2, EF-hand calcium binding domain [Homo |
| | | sapiens] |
| HG1018425 | NP_002893:NM_002902_1-19 | reticulocalbin 2, EF-hand calcium binding domain [Homo |
| | | sapiens] |
| HG1018426 | NP_002893:NM_002902_1-22 | reticulocalbin 2, EF-hand calcium binding domain [Homo |
| | | sapiens] |
| HG1018427 | NP_002893:NM_002902_1-18 | reticulocalbin 2, EF-hand calcium binding domain [Homo |
| | | sapiens] |
| HG1018428 | NP_002893:NM_002902_1-20 | reticulocalbin 2, EF-hand calcium binding domain [Homo |
| | | sapiens] |
| HG1018429 | NP_002893:NM_002902_1-21 | reticulocalbin 2, EF-hand calcium binding domain [Homo |
| | | sapiens] |
| HG1018430 | NP_002893:NM_002902_1-23 | reticulocalbin 2, EF-hand calcium binding domain [Homo |
| | | sapiens] |
| HG1018432 | NP_005133:NM_005142_1-19 | gastric intrinsic factor (vitamin B synthesis) [Homo sapiens] |
| HG1018433 | NP_005133:NM_005142_1-18 | gastric intrinsic factor (vitamin B synthesis) [Homo sapiens] |
| HG1018434 | NP_005133:NM_005142_1-20 | gastric intrinsic factor (vitamin B synthesis) [Homo sapiens] |
| HG1018435 | NP_005133:NM_005142_1-24 | gastric intrinsic factor (vitamin B synthesis) [Homo sapiens] |
| HG1018436 | NP_005133:NM_005142_1-16 | gastric intrinsic factor (vitamin B synthesis) [Homo sapiens] |
| HG1018437 | NP_005133:NM_005142_1-17 | gastric intrinsic factor (vitamin B synthesis) [Homo sapiens] |
| HG1018438 | NP_005133:NM_005142_1-14 | gastric intrinsic factor (vitamin B synthesis) [Homo sapiens] |
| HG1018440 | NP_005445:NM_005454_1-17 | cerberus 1 [Homo sapiens] |
| HG1018442 | NP_005555:NM_005564_1-18 | lipocalin 2 (oncogene 24p3) [Homo sapiens] |
| HG1018443 | NP_005555:NM_005564_1-20 | lipocalin 2 (oncogene 24p3) [Homo sapiens] |
| HG1018444 | NP_005555:NM_005564_1-15 | lipocalin 2 (oncogene 24p3) [Homo sapiens] |
| HG1018446 | NP_005690:NM_005699_1-29 | interleukin 18 binding protein isoform C precursor [Homo |
| | | sapiens] |
| HG1018447 | NP_005690:NM_005699_1-24 | interleukin 18 binding protein isoform C precursor [Homo |
| | | sapiens] |
| HG1018448 | NP_005690:NM_005699_1-28 | interleukin 18 binding protein isoform C precursor [Homo |
| | | sapiens] |
| HG1018450 | NP_006560:NM_006569_1-19 | cell growth regulator with EF hand domain 1 [Homo sapiens] |
| HG1018451 | NP_006560:NM_006569_1-18 | cell growth regulator with EF hand domain 1 [Homo sapiens] |
| HG1018452 | NP_006560:NM_006569_1-21 | cell growth regulator with EF hand domain 1 [Homo sapiens] |
| HG1018454 | NP_006856:NM_006865_1-15 | leukocyte immunoglobulin-like receptor, subfamily A |
| | | (without TM |
| HG1018456 | NP_036577:NM_012445_1-26 | spondin 2, extracellular matrix protein [Homo sapiens] |
| HG1018457 | NP_036577:NM_012445_1-25 | spondin 2, extracellular matrix protein [Homo sapiens] |
| HG1018458 | NP_036577:NM_012445_1-24 | spondin 2, extracellular matrix protein [Homo sapiens] |
| HG1018459 | NP_036577:NM_012445_1-28 | spondin 2, extracellular matrix protein [Homo sapiens] |
| HG1018461 | NP_055070:NM_014255_1-20 | transmembrane protein 4 [Homo sapiens] |
| HG1018462 | NP_055070:NM_014255_1-18 | transmembrane protein 4 [Homo sapiens] |
| HG1018463 | NP_055070:NM_014255_1-16 | transmembrane protein 4 [Homo sapiens] |
| HG1018465 | NP_055582:NM_014767_1-24 | sparc/osteonectin, cwcv and kazal-like domains proteoglycan |
| HG1018466 | NP_055582:NM_014767_1-19 | sparc/osteonectin, cwcv and kazal-like domains proteoglycan |
| HG1018467 | NP_055582:NM_014767_1-22 | sparc/osteonectin, cwcv and kazal-like domains proteoglycan |
| HG1018468 | NP_055582:NM_014767_1-20 | sparc/osteonectin, cwcv and kazal-like domains proteoglycan |
| HG1018469 | NP_055582:NM_014767_1-26 | sparc/osteonectin, cwcv and kazal-like domains proteoglycan |
| HG1018470 | NP_055582:NM_014767_1-21 | sparc/osteonectin, cwcv and kazal-like domains proteoglycan |
| HG1018472 | NP_055697:NM_014882_1-18 | Rho GTPase activating protein 25 isoform b [Homo sapiens] |
| HG1018474 | NP_056965:NM_015881_1-18 | dickkopf homolog 3 [Homo sapiens] |
| HG1018475 | NP_056965:NM_015881_1-19 | dickkopf homolog 3 [Homo sapiens] |
| HG1018476 | NP_056965:NM_015881_1-22 | dickkopf homolog 3 [Homo sapiens] |
| HG1018477 | NP_056965:NM_015881_1-16 | dickkopf homolog 3 [Homo sapiens] |
| HG1018478 | NP_056965:NM_015881_1-21 | dickkopf homolog 3 [Homo sapiens] |
| HG1018480 | NP_057603:NM_016519_1-26 | ameloblastin precursor [Homo sapiens] |
| HG1018481 | NP_057603:NM_016519_1-28 | ameloblastin precursor [Homo sapiens] |
| HG1018483 | NP_149439:NM_033183_1-18 | chorionic gonadotropin, beta polypeptide 8 recursor [Homo |
| | | sapiens] |
| HG1018484 | NP_149439:NM_033183_1-20 | chorionic gonadotropin, beta polypeptide 8 recursor [Homo |
| | | sapiens] |
| HG1018485 | NP_149439:NM_033183_1-16 | chorionic gonadotropin, beta polypeptide 8 recursor [Homo |
| | | sapiens] |
| HG1018487 | NP_644808:NM_139279_1-18 | multiple coagulation factor deficiency 2 [Homo sapiens] |
| HG1018488 | NP_644808:NM_139279_1-20 | multiple coagulation factor deficiency 2 [Homo sapiens] |
| HG1018489 | NP_644808:NM_139279_1-26 | multiple coagulation factor deficiency 2 [Homo sapiens] |
| HG1018490 | NP_644808:NM_139279_1-23 | multiple coagulation factor deficiency 2 [Homo sapiens] |
| HG1018492 | NP_660295:NM_145252_1-13 | similar to common salivary protein 1 [Homo sapiens] |
| HG1018493 | NP_660295:NM_145252_1-16 | similar to common salivary protein 1 [Homo sapiens] |
| HG1018494 | NP_660295:NM_145252_1-14 | similar to common salivary protein 1 [Homo sapiens] |
| HG1018495 | NP_660295:NM_145252_1-17 | similar to common salivary protein 1 [Homo sapiens] |
| HG1018497 | NP_689534:NM_152321_1-25 | hypothetical protein FLJ32115 [Homo sapiens] |
| HG1018498 | NP_689534:NM_152321_1-21 | hypothetical protein FLJ32115 [Homo sapiens] |
| HG1018500 | NP_689848:NM_152635_1-18 | oncoprotein-induced transcript 3 [Homo sapiens] |
| HG1018501 | NP_689848:NM_152635_1-16 | oncoprotein-induced transcript 3 [Homo sapiens] |
| HG1018502 | NP_689848:NM_152635_1-15 | oncoprotein-induced transcript 3 [Homo sapiens] |
| HG1018504 | NP_689968:NM_152755_1-21 | hypothetical protein MGC40499 [Homo sapiens] |
| HG1018506 | NP_766630:NM_173042_1-29 | interleukin 18 binding protein isoform A precursor [Homo |
| | | sapiens] |
| HG1018507 | NP_766630:NM_173042_1-24 | interleukin 18 binding protein isoform A precursor [Homo |
| | | sapiens] |
| HG1018508 | NP_766630:NM_173042_1-28 | interleukin 18 binding protein isoform A precursor [Homo |
| | | sapiens] |
| HG1018510 | NP_776214:NM_173842_1-23 | interleukin 1 receptor antagonist isoform 1 precursor [Homo |
| | | sapiens] |
| HG1018511 | NP_776214:NM_173842_1-25 | interleukin 1 receptor antagonist isoform 1 precursor [Homo |
| | | sapiens] |
| HG1018513 | NP_783165:NM_175575_1-32 | WFIKKN2 protein [Homo sapiens] |
| HG1018514 | NP_783165:NM_175575_1-34 | WFIKKN2 protein [Homo sapiens] |
| HG1018515 | NP_783165:NM_175575_1-29 | WFIKKN2 protein [Homo sapiens] |
| HG1018516 | NP_783165:NM_175575_1-30 | WFIKKN2 protein [Homo sapiens] |
| HG1018517 | NP_783165:NM_175575_1-27 | WFIKKN2 protein [Homo sapiens] |
| HG1018857 | 27482680:27482679_1-26 | similar to hypothetical protein 9330140G23 [Homo sapiens] |
| HG1018858 | 27482680:27482679_1-24 | similar to hypothetical protein 9330140G23 [Homo sapiens] |

### INDUSTRIAL APPLICABILITY

The polypeptide and modulator compositions and methods of the invention are useful in the diagnosis, treatment, and/or prevention of proliferative diseases, including cancer, inflammatory and immune or autoimmune diseases, neurodegenerative diseases, infectious diseases, metabolic diseases such as diabetes and iscliemia-related disorders.

1. A nucleic acid molecule comprising a first polynucleotide that comprises a first nucleotide sequence encoding an APO2L polypeptide chosen from a continuous sequence of:
(a) SEQ ID NOS: 16, or 18;
(b) a polynucleotide encoding a polypeptide comprising an amino acid sequence of SEQ ID NO:15 or 22;
(c) a complementary polynucleotide comprising a complementary nucleotide sequence that is complementary to the first nucleotide sequence of (a); and
(d) a biologically active fragment of any of (a)-(c);
wherein the nucleic acid molecule is an isolated molecule.

2-3. (canceled)

4. The nucleic acid molecule of claim 1, wherein the first nucleotide sequence is a biologically active fragment of SEQ ID NO:14 and consists essentially of SEQ ID NO:17.

5. The nucleic acid molecule of claim 1, wherein the first nucleotide sequence comprises SEQ ID NO:18.

6. The nucleic acid molecule of claim 1, further comprising a second polynucleotide.

7. The nucleic acid molecule of claim 6, wherein the second polynucleotide comprises a second nucleotide sequence encoding a secretory leader, and the secretory leader is a homologous or heterologous leader.

8. (canceled)

9. The nucleic acid molecule of claim 7, wherein the secretory leader is a secretory leader chosen from SEQ ID NOS:26-223.

10. A polypeptide comprising a first amino acid sequence, wherein the first amino acid sequence comprises a continuous sequence chosen from:
(a) SEQ ID NO:15 or 22;
(b) a sequence encoded by one of SEQ ID NOS:14, 16, or 18;
and
(c) an active fragment of (a) or (b); wherein the polypeptide is an isolated molecule.

11. The polypeptide of claim 10, wherein the polypeptide is present in a cell culture.

12-14. (canceled)

15. The polypeptide of claim 10, wherein the first amino acid sequence is an active fragment of SEQ ID NO:15 and consists essentially of an amino acid sequence of SEQ ID NO:21.

16. (canceled)

17. The polypeptide of claim 10, wherein the polypeptide further comprises a second amino acid sequence, and the second amino acid sequence is a secretory leader, the secretory leader is a homologous leader or a heterologous leader, and wherein the first and second amino acid sequences are operably linked.

18. (canceled)

19. The polypeptide of claim 17 wherein the secretory leader sequence is a heterologous leader sequence, and the heteroloqous leader sequence is chosen from SEQ ID NOS:26-223.

20. (canceled)

21. A vector comprising the nucleic acid molecule of claim 1 and a promoter that regulates the expression of the nucleic acid molecule.

22-25. (canceled)

26. A recombinant host cell comprising a cell and the nucleic acid molecule of claim 1.

27-41. (canceled)

42. A method of producing a recombinant host cell comprising:
(a) providing a vector that comprises the nucleic acid molecule of claim 1; and
(b) allowing a cell to come into contact with the vector to form a recombinant host cell transfected with the nucleic acid molecule.

43. A method of producing a polypeptide comprising:
(a) providing the nucleic acid of claim 1; and
(b) expressing the nucleic acid molecule in an expression system to produce the polypeptide.

44-47. (canceled)

48. A polypeptide produced by the method of claim 43.

49-52. (canceled)

53. The polypeptide of claim 10 wherein the polypeptide further comprises at least one fusion partner.

54. (canceled)

55. A method of inhibiting tumor growth comprising:
(a) providing a composition that comprises a polypeptide and a carrier; and
(b) contacting the tumor with the compositions
wherein the Polypeptide comprises a first amino acid sequence, and the first amino acid sequence comprises a continuous sequence chosen from: (i) SEQ ID NOS:15 or 22: (ii) a sequence encoded by one of SEQ ID NOS:14, 16, or 18; and (iii) an active fraqment of (i) or (ii).

56-73. (canceled)

74. A method of treating a tumor in a subject comprising:
(a) providing a first composition comprising the polypeptide of claim 10;
(b) providing a second composition comprising an anti-cancer agent different from the polypeptide of claim 10; and
(c) administering the first and second compositions to the subject.

75-90. (canceled)

91. The polypeptide of claim 10, wherein the first amino acid sequence comprises SEQ ID NO:22.
